(19) United States
(12) Patent Application Publication
(10) Pub. No.: US 2006/0264419 A1
(43) Pub. Date: Nov. 23, 2006

(54) Substituted pyrazoles

(76) Inventors: Kai Schiemann, Seeheim-Jugenheim (DE); Karl-August Ackermann, Ober-Ramstadt (DE); Michael Arlt, Alsbach (DE); Dirk Finsinger, Munich (DE); Christoph Van Amsterdam, Darmstadt (DE); Gerd Bartoszyk, Weiterstadt (DE); Christoph Seyfried, Seeheim-Jugenheim (DE); Oliver Schadt, Rodenbach (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/552,065

(22) PCT Filed: Mar. 8, 2004
(86) PCT No.: PCT/EP04/02353

§ 371 Date: Oct. 5, 2005

(30) Foreign Application Priority Data
Apr. 5, 2003 (DE) 103 15 572.4

(51) Int. Cl. A61K 31/551; A61K 31/5377; A61K 31/496; A61K 31/4439; A61K 31/416

(52) U.S. Cl. 514/218; 514/235.2; 514/253.09; 514/406; 514/254.05

(57) ABSTRACT

The compounds of the formula (I) and salts and solvates thereof, in which X, R¹, R², R³, R⁴ and R⁵ have the meanings indicated in Claim (1), are suitable as ligands of 5 HT receptors.

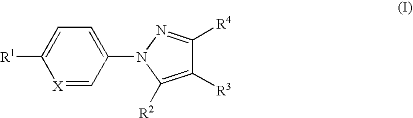

The invention relates to the use of the compounds of the formula I 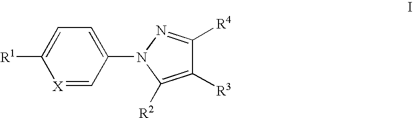
in which
X denotes CH or N,
R¹ denotes H, A, Hal, (CH₂)ₙHet, (CH₂)ₙAr, cycloalkyl having 3 to 7 C atoms, CF₃, NO₂, CN, C(NH)NOH or OCF₃,
R² denotes (CH₂)ₙHet, (CH₂)ₙAr, cycloalkyl having 3 to 7 C atoms or CF₃,
R³, R⁴ denote H or an organic radical, in particular (CH₂)ₙCO₂R⁵, (CH₂)ₙCOHet, (CH₂)ₙCON(R⁵)₂, (CH₂)ₙCOO(CH₂)ₙHet, CHO, (CH₂)ₙOR⁵, (CH₂)ₙHet, (CH₂)ₙN(R⁵)₂, CH═N—OA, CH₂CH═N—OA, (CH₂)ₙNHOA, (CH₂)ₙN(R⁵)Het, (CH₂)ₙCH═N-Het, (CH₂)ₙOCOR⁵, (CH₂)ₙN(R⁵)CH₂CH₂OR⁵, (CH₂)ₙN(R⁵)CH₂CH₂OCF₃, (CH₂)ₙN(R⁵)C(R⁵)HCOOR⁵, (CH₂)ₙN(R⁵)CH₂COHet, (CH₂)ₙN(R⁵)CH₂Het, (CH₂)ₙN(R⁵)CH₂CH₂Het, (CH₂)ₙN(R⁵)CH₂CH₂N(R⁵)CH₂COOR⁵, (CH₂)ₙN(R⁵)CH₂CH₂OR⁵, (CH₂)ₙN(R⁵)CH₂CH₂N(R⁵)₂, CH═CHCOOR⁵, CH═CHCH₂NR⁵Het, CH═CHCH₂N(R⁵)₂, CH═CHCH₂OR⁵, CH═CHCH₂Het, (CH₂)ₙN(R⁵)Ar, (CH₂)ₙN(COOR⁵)COOR⁵, (CH₂)ₙN(CONH₂)COOR⁵, (CH₂)ₙN(CONH₂)CONH₂, (CH₂)ₙN(CH₂COOR⁵)COOR⁵, (CH₂)ₙN(CH₂CONH₂)COOR⁵, (CH₂)ₙN(CH₂CONH₂)CONH₂, (CH₂)ₙCHR⁵COR⁵, (CH₂)ₙCHR⁵COOR⁵, (CH₂)ₙCHR⁵CH₂OR⁵, where in each case one of the radicals R³ or R⁴ has the meaning H,
R⁵ denotes H or A
A denotes straight-chain or branched alkyl or cycloalkyl having 2 to 4 C atoms, having 1 to 10 C atoms, alkenyl having 2 to 10 C atoms, alkoxyalkyl having 2 to 10 C atoms or cycloalkyl having 4 to 7 C atoms, each of which is unsubstituted or substituted by Hal or CN,
Het denotes an organic radical containing hetero atoms, in particular a saturated, unsaturated or aromatic mono- or bicyclic heterocyclic radical having 1 to 15 C atoms which is unsubstituted or mono- or polysubstituted by A and/or Hal or a linear radical having 1 to 15 C atoms containing one or two hetero atoms,
Ar denotes an aromatic organic radical, in particular a phenyl radical which is unsubstituted or mono- or polysubstituted by A and/or Hal, OR⁵, OOCR⁵, COOR⁵, CON(R⁵)₂, CN, NO₂, NH₂, NHCOR⁵, CF₃ or SO₂CH₃,
n denotes 0, 1, 2, 3, 4 or 5
   and
Hal denotes F, Cl, Br or I,
   and salts and solvates, enantiomers and racemates thereof, in particular physiologically tolerated salts and solvates thereof, for the treatment and prophylaxis of diseases which can be influenced by the binding of the compounds of the formula I to 5 HT receptors. The invention had the object of finding compounds which can be used for the preparation of medicaments. It has been found that the compounds of the formula I and salts and solvates thereof have very valuable pharmacological properties and are well tolerated. The invention relates, in particular, to the compounds mentioned in the examples, which have the properties and potential uses of the compounds of the formula I that are outlined in the present application. Similar compounds are disclosed, for example, in DE 2201889, DE 2258033 or DE 2906252.

In particular, the compounds of the formula I according to the invention are suitable as ligands of 5 HT receptors, in particular of 5 HT2A and/or 5HT2C receptors, and can be used in human and veterinary medicine for the prophylaxis and treatment of various diseases of the central nervous system, such as, for example, schizophrenia, depression, dementia, Parkinson's disease, Alzheimer's disease, Lewy bodies dementia, Huntington's, Tourette's syndrome, anxiety, learning and memory impairments, neurodegenerative diseases and other cognitive impairments, as well as nicotine dependence and pain.

The compounds of the formula I and/or physiologically acceptable salts or solvates thereof are particularly preferably used for the preparation of a medicament for the prophylaxis and/or treatment of psychoses, neurological disorders, amyotrophic lateral sclerosis, eating disorders, such as bulimia, anorexia nervosa, of premenstrual syndrome and/or for positively influencing obsessive-compulsive disorder (OCD).

It has been found that the compounds of the formula I and physiologically acceptable salts and solvates thereof, while being well tolerated, have valuable pharmacological properties since they have actions on the central nervous system. The compounds have strong affinity to 5-HT_{2A} receptors, they furthermore exhibit 5-HT_{2A} receptor-antagonistic properties.

Particular preference is therefore given to the use of the compounds of the formula I and/or physiologically acceptable salts and solvates thereof for the preparation of a medicament having a 5-HT receptor-antagonistic action.

For in-vitro detection of the affinity to 5-HT_{2A} receptors, the following test (Example A1), for example, can be used. The 5-HT_{2A} receptors are exposed both to [³H]ketanserine (a substance which is known for its affinity to the receptor) and also to the test compound. The decrease in the affinity of [³H]ketanserine to the receptor is an indication of the affinity of the test substance to the 5-HT_{2A} receptor. The detection is carried out analogously to the description by J. E. Leysen et al., Molecular Pharmacology, 1982, 21: 301-314, or as also described, for example, in EP 0320983.

The efficacy of the compounds according to the invention as 5-HT_{2A} receptor antagonists can be measured in vitro analogously to W. Feniuk et al., Mechanisms of 5-hydroxytryptamine-induced vasoconstriction, in: The Peripheral Actions of 5-Hydroxytryptamine, ed. Fozard JR, Oxford University Press, New York, 1989, p. 110. Thus, the contractility of the rat tail artery caused by 5-hydroxytryptamine is mediated by 5-HT_{2A} receptors. For the test system, vessel rings prepared from the ventral rat tail artery are subjected to perfusion in an organ bath containing an oxygen-saturated solution. By introducing increasing concentrations of 5-hydroxytryptamine into the solution, a response is obtained to the cumulative concentration of 5-HT. The test compound is then added to the organ bath in suitable concentrations, and a second concentration curve for 5-HT is measured. The strength of the test compound in shifting the 5-HT-induced concentration curve to higher 5-HT concentrations is a measure of the 5-HT_{2A} receptor-antagonistic property in vitro.

The 5-HT_{2A}-antagonistic property can be determined in vivo analogously to M. D. Serdar et al., Psychopharmacology, 1996, 128: 198-205.

The compounds of the formula I are therefore suitable both in veterinary and in human medicine for the treatment of functional disorders of the central nervous system and of inflammation. They can be used for the prophylaxis of and for combating the consequences of cerebral infarction phenomena (apoplexia cerebri), such as strokes and cerebral ischaemia, and for the treatment of extrapyramidal motor side effects of neuroleptics and of Parkinson's disease, for the acute and symptomatic therapy of Alzheimer's disease and for the treatment of amyotrophic lateral sclerosis. They are likewise suitable as therapeutic agents for the treatment of brain and spinal cord traumas. In particular, however, they are suitable as medicament active ingredients for anxiolytics, antidepressants, antipsychotics, neuroleptics, antihypertonics and/or for positively influencing obsessive-compulsive disorder (OCD; for example WO 9524194), anxiety states and physiological changes associated with anxiety states, such as, for example, tachycardia, tremor or sweating (for example EP 319962), panic attacks, psychoses, schizophrenia, anorexia, delusional obsessions, agoraphobia, migraine, Alzheimer's disease, sleep disorders, including sleep apnoea, tardive dyskinesia, learning disorders, age-dependent memory disorders, eating disorders, such as bulimia, drugs misuse, such as, for example, of alcohol, opiates, nicotine, psychostimulants, such as, for example, cocaine or amphetamines (for example U.S. Pat. No. 6,004,980), sexual dysfunctions, conditions of pain of all types and fibromyalgia (for example WO 9946245). The compounds of the formula I are suitable for the treatment of extrapyramidal side effects (EPS) in neuroleptic drug therapy. EPS is characterised by Parkinson's-like syndromes, acathisia and dystonic reactions (for example EP 337136). They are furthermore suitable for the treatment of anorexia nervosa, angina, Reynaud's, coronary vasospasms, in the prophylaxis of migraine (for example EP 208235), pain and neuralgia (for example EP 320983), for the treatment of Rett syndrome with autistic traits, of Asperger's syndrome, of autism and autistic disorders, in concentration deficit states, developmental disorders, hyperactivity states with mental underdevelopment and stereotypical behaviour states (for example WO 9524194).

They are furthermore suitable for the treatment of endocrine diseases, such as hyperprolactinaemia, furthermore in vasospasms, thrombotic diseases (for example WO 9946245), hypertension and gastrointestinal diseases.

They are furthermore suitable for the treatment of cardiovascular diseases and extrapyramidal symptoms, as described in WO 99/11641 on page 2, line 24-30.

The compounds according to the invention are furthermore suitable for reducing the intraocular pressure and for the treatment of glaucoma.

They are also suitable for the prophylaxis and treatment of poisoning phenomena on administration of ergovaline to animals.

The compounds are furthermore suitable for the treatment of diseases of the cardiovascular system (WO 99/11641, page 3, line 14-15). The compounds according to the invention can also be employed together with other active ingredients in the treatment of schizophrenia. Suitable other active ingredients are the compounds mentioned in WO 99/11641 on page 13, line 20-26.

Other compounds which likewise exhibit 5-HT₂-antagonistic actions are described, for example, in EP 0320983.

WO 99/11641 describes phenylindole derivatives having 5-HT₂-antagonistic properties.

However, none of the above-mentioned documents describes the use according to the invention of the compounds of the formula I as ligands of 5 HT receptors.

The compounds of the formula I can be employed as medicament active ingredients in human and veterinary medicine. They can furthermore be employed as intermediates for the preparation of further medicament active ingredients.

The invention accordingly relates to the use of the compounds of the formula I in human and animal medicine.

The invention furthermore relates to the novel compounds of the formula I.

The compounds of the formula I are preferably prepared by firstly reacting a compound of the formula II 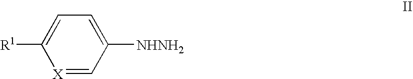
or acid-addition salts thereof
in which
R¹ and X have the meanings indicated above,
with a compound of the formula III 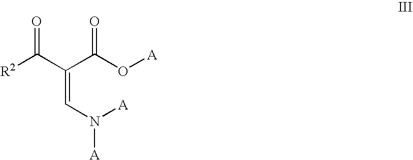
in which
A and R² have the meanings indicated above, to give a compound of the formula IA 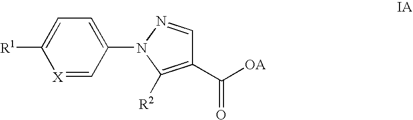
or by reacting a compound of the formula II 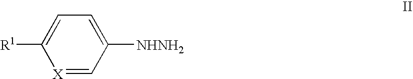
or acid-addition salts thereof
in which
R¹ and X have the meanings indicated above,
with a compound of the formula IV 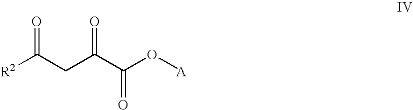
in which
A and R² have the meanings indicated above, to give a compound of the formula IB 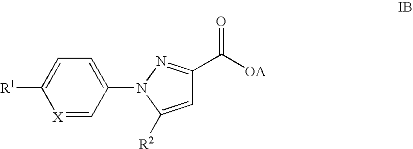
and then converting the compounds of the formulae IA and IB into the further compounds of the formula I by conventional methods.

In particular, the compounds of the formula IA and IB can be converted, by using reducing agents, such as, for example, lithium aluminium hydride, into the corresponding alcohols of the formulae IC and ID 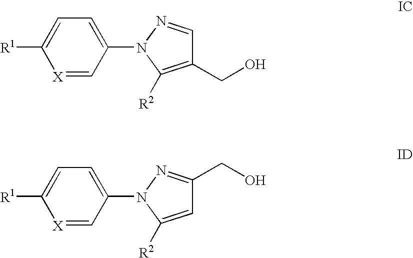
which can be oxidised, for example using MnO₂, to the compounds IE and IF.

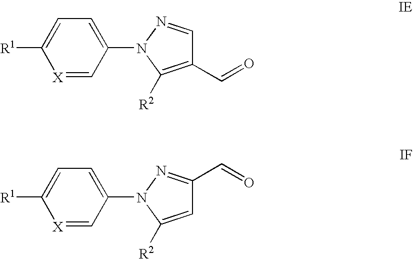

The compounds of the formulae IE and IF can themselves be aminated by known processes using corresponding nucleophiles, such as, for example, nitrogen bases, in particular hydroxylamine, O-methylhydroxylamine, morpholine, piperidine, piperazine, N-methylpiperazine, 4-methylpiperazin-1-ylamine, pyrrolidine, pyrazolidine or imidazolidine, optionally in the presence of a reducing agent, such as sodium triacetoxyborohydride, or converted into the corresponding imines. Furthermore, the compounds of the formulae IE and IF can be converted, by Wittig reaction with methoxy-methyltriphenylphosphonium salts, into the corresponding enol ethers, which can be converted into the homologised aldehydes IG and IH 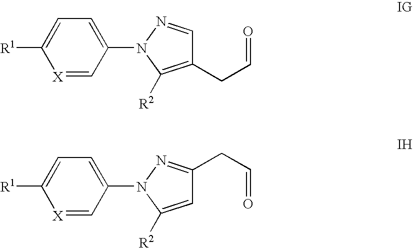
by treatment with an acid. The compounds of the formula IG and IH can be converted into the further compounds of the formula I analogously to the compounds of the formulae IE and IF.

Solvates of the compounds of the formula I are taken to mean adductions of inert solvent molecules onto the compounds of the formula I which form owing to their mutual attractive force. Solvates are, for example, mono- or dihydrates or alcoholates.

Above and below, the radicals X, A, Ar, Het, n, R¹, R², R³, R⁴ and R⁵ have the meanings indicated for the formula I, unless expressly stated otherwise.

X preferably denotes CH.

R¹ preferably stands for A, Hal, (CH₂)ₙHet or (CH₂)ₙAr, in particular for A, (CH₂)ₙHet or (CH₂)ₙAr. R¹ very particularly preferably denotes phenyl, 2-, 3- or 4-cyanophenyl, 2-, 3- or 4-fluorophenyl, 2-, 3- or 4-methyl-, ethyl-, n-propyl- or n-butylphenyl, 2,3-, 2,4-, 2,5-, 2,6-, 3,4-, 3,5- or 3,6-difluoro-, dichloro- or dicyanophenyl, 3,4,5-trifluorophenyl, 3,4,5-trimethoxy- or triethoxyphenyl, thiophen-2-yl or thiophen-3-yl or 1-, 2- or 3-pyrrolyl.

R² preferably denotes (CH₂)ₙHet, (CH₂)ₙNHA, (CH₂)ₙNHCH₂Het or (CH₂)ₙAr, in particular (CH₂)ₙHet, (CH₂)ₙNHA, (CH₂)ₙNHCH₂Het. R² very particularly preferably denotes phenyl, 2-, 3- or 4-cyanophenyl, 2-, 3- or 4-fluorophenyl, 2-, 3- or 4-methyl-, ethyl-, n-propyl- or n-butylphenyl, 2,3-, 2,4-, 2,5-, 2,6-difluoro- or dicyanophenyl, thiophen-2-yl or thiophen-3-yl, 2-, 3- or 4-pyridyl, 2-, 4- or 5-oxazolyl, 2-, 4- or 5-thiazolyl, quinolinyl, isoquinolinyl, 2- or 4-pyridazyl, 2-, 4- or 5-pyrimidyl, 2- or 3-pyrazinyl, 2- or 3-furyl.

If R³ denotes H, R⁴ preferably has the meaning (CH₂)ₙCO₂R⁵, (CH₂)ₙ-Het, (CH₂)ₙNHA, (CH₂)ₙNHCH₂-Het, (CH₂)ₙCO-Het, CHO, CH₂OR⁵, (CH₂)ₙN(R⁵)₂ or CH═N—OA, but in particular (CH₂)ₙCO₂R⁵, (CH₂)ₙCO-Het, CHO, CH═N-OA or (CH₂)ₙ-Het. If R⁴ denotes H, R³ preferably has the meaning (CH₂)ₙCO₂R⁵, (CH₂)ₙCO-Het, CHO, CH₂OR⁵, (CH₂)ₙ-Het, (CH₂)ₙN(R⁵)₂ or CH═N—OA, (CH₂)ₙN(R⁵)Het, (CH₂)ₙN(R⁵)CH₂CH₂OR⁵, (CH₂)ₙN(R⁵)CH₂Het, (CH₂)ₙN(R⁵)CH₂CH₂Het, (CH₂)ₙN(R⁵)CH₂CH₂N(R⁵)₂, CH═CHCH₂NR⁵Het, CH═CHCH₂N(R⁵)₂, CH═CHCH₂OR⁵, CH═CHCH₂Het or (CH₂)ₙN(R⁵)Ar, but in particular (CH₂)ₙHet, (CH₂)ₙN(R⁵)₂, (CH₂)ₙN(R⁵)Het, (CH₂)ₙN(R⁵)CH₂CH₂OR⁵, (CH₂)ₙN(R⁵)CH₂Het, (CH₂)ₙN(R⁵)CH₂CH₂Het, (CH₂)ₙN(R⁵)CH₂CH₂N(R⁵)₂, CH═CHCH₂NR⁵Het, CH═CHCH₂N(R⁵)₂, CH═CHCH₂OR⁵, CH═CHCH₂Het, (CH₂)ₙN(R⁵)Ar. Further preferred meanings of the radicals R³ arise from the examples. R⁴ particularly preferably denotes H.

R⁵ preferably has the meaning A.

A preferably denotes alkyl, is preferably unbranched and has 1, 2, 3, 4, 5, 6, 7, 8, 9 or 10 C atoms, preferably 1, 2, 3, 4, 5 or 6 C atoms, and preferably denotes methyl, ethyl, n-or propyl, furthermore preferably isopropyl, butyl, isobutyl, sec-butyl or tert-butyl, but also n-pentyl, neopentyl, isopentyl or n-hexyl. Particular preference is given to methyl, ethyl, n-propyl, isopropyl, n-butyl, n-pentyl, n-hexyl or n-decyl.

A furthermore preferably has the meaning of the (CH₂)ₘOCH₃ or (CH₂)ₘC₂H₅ group, in which m denotes 2, 3, 4, 5 or 6, but in particular 2.

If A denotes alkenyl, it preferably stands for allyl, 2- or 3-butenyl, isobutenyl, sec-butenyl, furthermore preferably 4-pentenyl, isopentenyl or 5-hexenyl.

Het is preferably an aromatic and in particular saturated heterocyclic radical which is unsubstituted or substituted by A. Het preferably denotes 1-piperidyl, 1-piperazyl, 1-(4-methyl)piperazyl, 4-methylpiperazin-1-ylamine, 4-morpholinyl, 1-pyrrolidinyl, 1-pyrazolidinyl 1-(2-methyl)pyrazolidinyl, 1-imidazolidinyl or 1-(3-methyl)imidazolidinyl, thiophen-2-yl or thiophen-3-yl, 2-, 3- or 4-pyridyl, which may be unsubstituted or substituted by one or more CN group, 2-, 4- or 5-oxazolyl, 2-, 4- or 5-thiazolyl, quinolinyl, isoquinolinyl, 2- or 4-pyridazyl, 2-, 4- or 5-pyrimidyl, 2- or 3-pyrazinyl. Het furthermore preferably denotes a radical from the following table:

| | |
|---|---|
| 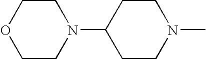 | 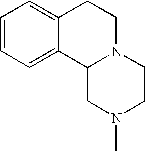 |
| 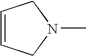 | 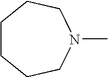 |
| 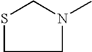 | 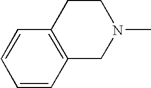 |
| 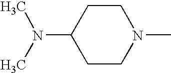 | 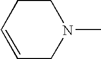 |
| 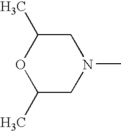 | 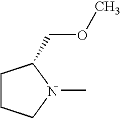 |
| 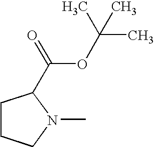 | 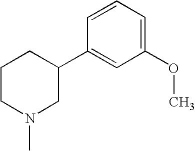 |
| 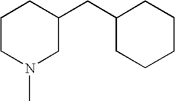 | 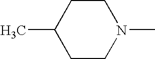 |
| 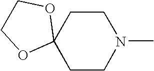 | 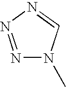 |
| 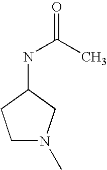 | 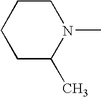 |
| 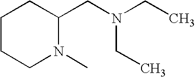 | 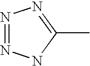 |
| 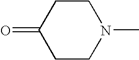 | 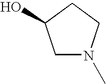 |
| 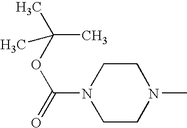 | 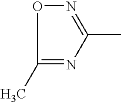 |
| 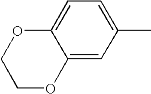 | 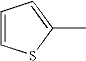 |
| 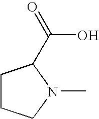 | 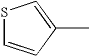 |
| 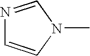 | 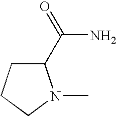 |
| 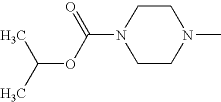 | 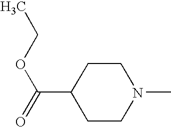 |
| 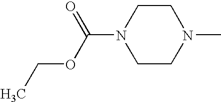 | 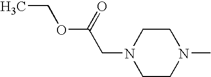 |
| 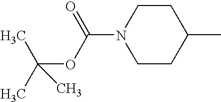 | 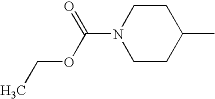 |
| 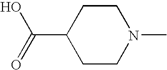 | 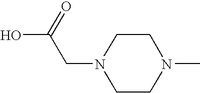 |
| 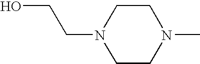 | 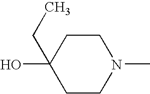 |
| 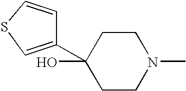 | 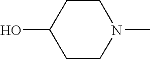 |
| 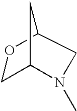 | 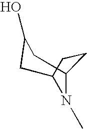 |
| 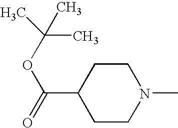 | 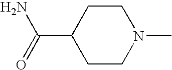 |
| 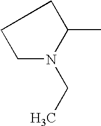 | 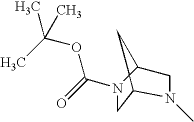 |
| 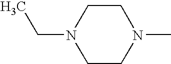 | 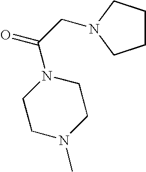 |
| 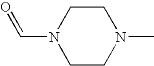 | 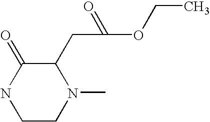 |
| 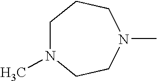 | 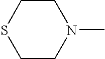 |
| 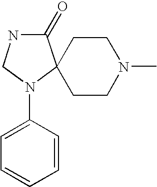 | 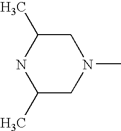 |
| 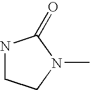 | 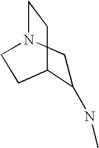 |
| 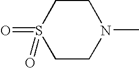 | 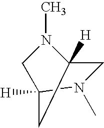 |
| 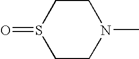 | 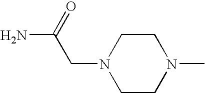 |
| 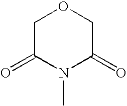 | 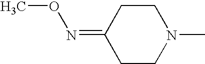 |
| 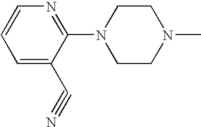 | 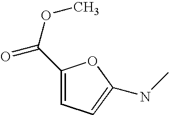 |
| 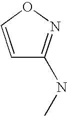 | 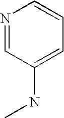 |
| 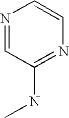 | 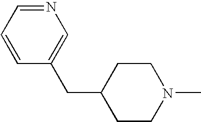 |
| 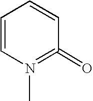 | 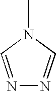 |
| 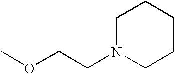 | 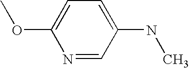 |
| 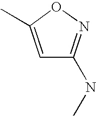 | 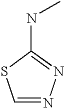 |
| 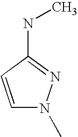 | 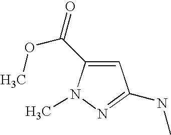 |
| 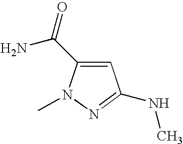 | |
| 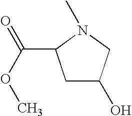 | 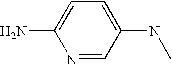 |
| 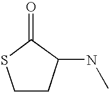 | 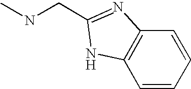 |
| 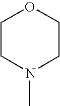 | 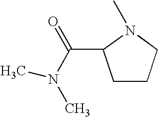 |
| 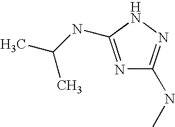 | 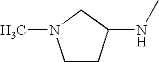 |
| 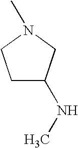 | 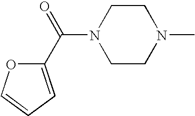 |
| 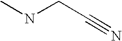 | 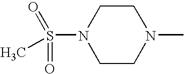 |
| 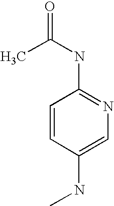 | 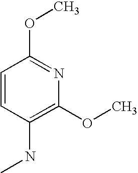 |
| 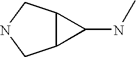 | 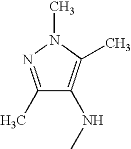 |
| 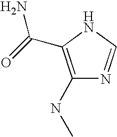 | 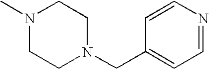 |
| 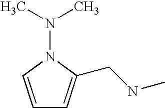 | 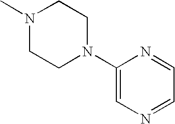 |
| 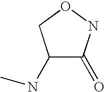 | 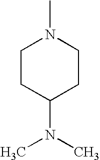 |
| 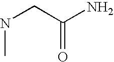 | 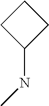 |
| 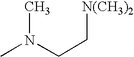 | 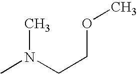 |
| 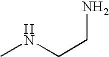 | 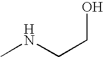 |
| 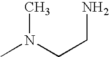 | 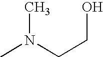 |

Het particularly preferably denotes one of the following radicals:

| | |
|---|---|
| 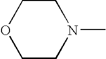 | 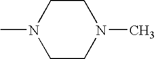 |
| 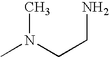 | 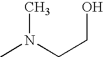 |
| 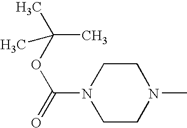 | 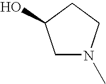 |
| 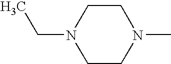 | 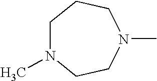 |
| 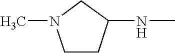 | 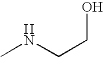 |
| 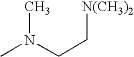 | 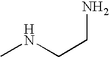 |

Ar preferably denotes a phenyl radical which is unsubstituted or substituted by Hal, OH, CN, NO₃ NH₂, NHCOCH₃, COOCH₃ CONH₂ or CF₃. Ar is preferably substituted in the 4- or 3-position.

n preferably denotes 0, 1 or 2, in particular 0 or 1.

Cycloalkyl preferably has 3-7 C atoms and preferably stands for cyclopropyl and cyclobutyl, furthermore preferably for cyclopentyl or cyclohexyl, furthermore also for cycloheptyl, particularly preferably cyclopentyl.

Hal preferably denotes F, Cl or Br, but also I.

If the compounds of the formula I has one or more chiral C atoms, the present invention relates to the enantiomers, diastereomers and mixtures thereof.

Throughout the invention, all radicals which occur more than once may be identical or different, i.e. are independent of one another.

Accordingly, the invention relates, in particular, to the compounds of the formula I in which at least one of the said radicals has one of the preferred meanings indicated above. Some preferred groups of compounds can be expressed by the following sub-formulae I1 to I9, which conform to the formula I and in which the radicals not designated in greater detail have the meaning indicated for the formula I, but in which

in I1

R¹ denotes (CH₂)ₙHet or (CH₂)ₙAr;
   in I2
R¹ denotes (CH₂)ₙHet or (CH₂)ₙAr
R² denotes (CH₂)ₙAr;
   in I3
R¹ denotes (CH₂)ₙAr;
R² denotes (CH₂)ₙAr;
   in I4
R¹ denotes (CH₂)ₙHet or (CH₂)ₙAr
R² denotes (CH₂)ₙAr
R⁴ denotes H
R³ denotes (CH₂)ₙ-Het, (CH₂)ₙNHA, (CH₂)ₙNHCH₂-Het, (CH₂)ₙCO₂R⁵, (CH₂)ₙCO-Het, CHO, CH₂OR⁵, (CH₂)ₙ-Het, (CH₂)ₙN(R⁵)₂ or CH═N—OA;
   in I5
R¹ denotes (CH₂)ₙHet or (CH₂)ₙAr
R² denotes (CH₂)ₙAr
R⁴ denotes H
R³ denotes (CH₂)ₙ-Het, (CH₂)ₙNHA, (CH₂)ₙNHCH₂-Het, (CH₂)ₙCO₂R⁵ (CH₂)ₙCO-Het, CHO, CH₂OR⁵, —(CH₂)ₙ-Het, (CH₂)ₙN(R⁵)₂ or CH═N—OA
R⁵ denotes-H, methyl, ethyl, n-propyl, isopropyl, n-butyl, n-pentyl, n-hexyl or n-decyl;
   in I6
R¹ denotes (CH₂)ₙHet or (CH₂)ₙAr
R² denotes (CH₂)ₙAr
R⁴ denotes H
R³ denotes (CH₂)ₙ-Het, (CH₂)ₙNHA, (CH₂)ₙNHCH₂-Het, (CH₂)ₙCO₂R⁵, (CH₂)ₙCO-Het, CHO, CH₂OR⁵, (CH₂)ₙ-Het, (CH₂)ₙN(R⁵)₂ or CH═N—OA
R⁵ denotes H, methyl, ethyl, n-propyl, isopropyl, n-butyl, n-pentyl, n-hexyl or n-decyl
n denotes 0, 1 or 2;
   in I7
R¹ denotes (CH₂)ₙHet or (CH₂)ₙAr
R² denotes (CH₂)ₙAr
R³ denotes H
R⁴ denotes (CH₂)ₙCO₂R⁵, (CH₂)ₙCO-Het, CHO, CH₂OR⁵, (CH₂)ₙ-Het, (CH₂)ₙN(R⁵)₂ or CH═N—OA;
   in I8
R¹ denotes (CH₂)ₙHet or (CH₂)ₙAr
R² denotes (CH₂)ₙAr
R³ denotes H
R⁴ denotes (CH₂)ₙCO₂R⁵, (CH₂)ₙCO-Het, CHO, CH₂OR⁵, (CH₂)ₙ-Het, (CH₂)ₙN(R⁵)₂ or CH═N—OA
R⁵ denotes H, methyl, ethyl, n-propyl, isopropyl, n-butyl, n-pentyl, n-hexyl or n-decyl;
   in I9
R¹ denotes (CH₂)ₙHet or (CH₂)ₙAr
R² denotes (CH₂)ₙAr
R³ denotes H
R⁴ denotes (CH₂)ₙCO₂R⁵, (CH₂)ₙCO-Het, CHO, CH₂OR⁵, (CH₂)ₙ-Het, (CH₂)ₙN(R⁵)₂ or CH═N—OA
R⁵ denotes H, methyl, ethyl, n-propyl, isopropyl, n-butyl, n-pentyl, n-hexyl or n-decyl
n denotes 0, 1 or 2;

Very particular preference is given to the compounds of the formulae a to o:

| | |
|---|---|
| [1-biphenyl-4-yl-5-(2-fluorophenyl)-1H-pyrazol-4-ylmethyl]- | (a) |
| (4-methylpiperazin-1-yl)amine | |
| 4-{2-[1-biphenyl-4-yl-5-(2-fluorophenyl)-1H-pyrazol-4-yl]- | (b) |
| ethyl}morpholine | |
| 4-{3-[1-biphenyl-4-yl-5-(2-fluorophenyl)-1H-pyrazol-4-yl]- | (c) |
| allyl}morpholine | |
| 1-[1-biphenyl-4-yl-5-(2-fluorophenyl)-1H-pyrazol-4-ylmethyl]- | (d) |
| pyrrolidin-3-ol | |
| 1-[1-(4′-fluorobiphenyl-4-yl)-5-(2-fluorophenyl)-1H-pyrazol- | (e) |
| 4-ylmethyl]-4-methylpiperazine | |
| 1-[5-(2-fluorophenyl)-1-(4-thiophen-3-ylphenyl)-1H-pyrazol- | (f) |
| 4-ylmethyl]-4-methylpiperazine | |
| 1-[5-furan-2-yl-1-(4-thiophen-3-ylphenyl)-1H-pyrazol-4-ylmethyl]- | (g) |
| 4-methylpiperazine | |
| N¹-[1-biphenyl-4-yl-5-(2-fluorophenyl)-1H-pyrazol-4-ylmethyl]- | (h) |
| ethane-1,2-diamine | |
| 2-{[1-biphenyl-4-yl-5-(2-fluorophenyl)-1H-pyrazol-4-ylmethyl]- | (i) |
| amino}ethanol | |
| [1-biphenyl-4-yl-5-(2-fluorophenyl)-1H-pyrazol-4-ylmethyl]- | (j) |
| (2-methoxyethyl)amine | |
| 2-{[1-biphenyl-4-yl-5-(2-fluorophenyl)-1H-pyrazol-4-ylmethyl]- | (k) |
| methylamino}ethanol | |
| 1-[1-biphenyl-4-yl-5-(2-fluorophenyl)-1H-pyrazol-4-ylmethyl]- | (l) |
| 4-methyl-[1,4]diazepam | |
| 1-[1-(4′-fluorobiphenyl-4-yl)-5-phenyl-1H-pyrazol-4-ylmethyl]- | (m) |
| 4-methylpiperazine | |
| 1-[5-(2-fluorophenyl)-1-(4-pyrrol-1-ylphenyl)-1H-pyrazol-4- | (n) |
| ylmethyl]-4-methylpiperazine | |
| [1-biphenyl-4-yl-5-(2-fluorophenyl)-1H-pyrazol-4-ylmethyl]- | (o) |
| methyl-(1-methylpyrrolidin-3-yl)amine | |

The compounds of the formula I and also the starting materials for their preparation are, in addition, prepared by methods known per se, as described in the literature (for example in the standard works, such as Houben-Weyl, Methodn der organischen Chemie [Methods of Organic Chemistry], Georg-Thieme-Verlag, Stuttgart), to be precise under reaction conditions which are known and suitable for the said reactions. Use can also be made here of variants known per se which are not mentioned here in greater detail.

The compound of the formula III is preferably obtained by reaction of compounds of the formula V 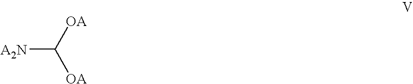
in which A has the meaning indicated above,
with compounds of the formula VI 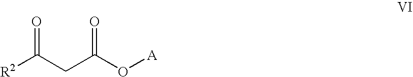
in which R² and A have the meaning indicated above,
under conditions known for such reactions.

The starting materials can, if desired, also be formed in situ by not isolating them from the reaction mixture, but instead immediately converting them further into the compounds of the formula I.

On the other hand, it is possible to carry out the reaction stepwise.

The starting materials of the formulae II, III and IV are generally known. If they are not known, they can be prepared by methods known per se.

Specifically, the reactions of the compounds of the formula II with the compounds of the formula III and the compounds of the formula IV are carried out in the presence or absence of a preferably inert solvent at temperatures between about −20 and about 150°, preferably between 20 and 100°.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); nitro compounds, such as nitromethane or nitrobenzene; esters, such as ethyl acetate, or mixtures of the said solvents.

The pH necessary for the reaction can be set in accordance with pH values selected for similar reactions of carbonyl compounds with amino compounds. The pH is preferably pre-specified through the use of the particular acid-addition salt, preferably a hydrogen halide addition salt, of the compound of the formula II, i.e. there is no additional addition of a base or acid to the reaction mixture. Preferred acid-addition salts are hydrochlorides or hydrobromides

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, sulfamic acid, furthermore organic acids, in particular aliphatic, alicyclic, araliphatic, aromatic or heterocyclic mono- or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, laurylsulfuric acid. Salts with physiologically unacceptable acids, for example picrates, can be used for the isolation and/or purification of the compounds of the formula I.

On the other hand, if desired, the free bases of the formula I can be liberated from their salts using bases (for example sodium hydroxide, potassium hydroxide, sodium carbonate or potassium carbonate).

The invention preferably relates to the use of the compounds of the formula I and/or physiologically acceptable salts and/or solvates thereof for the preparation of pharmaceutical compositions for the treatment or prophylaxis of diseases which can be influenced by the binding of the compounds of the formula I to 5 HT receptors, in particular by non-chemical methods. In this case, they can be converted into a suitable dosage form together with at least one solid, liquid and/or semi-liquid excipient or adjuvant and, if desired, in combination with one or more further active ingredients.

The invention furthermore relates to pharmaceutical compositions comprising at least one compound of the formula I and/or one of its physiologically acceptable salts and/or solvates for the treatment or prophylaxis of diseases which are influenced by the binding of the compounds of the formula I to 5 HT receptors.

These compositions can be used as medicaments in human or veterinary medicine. Suitable-excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc, Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, suitable for topical application are ointments, creams or powders. The novel compounds may also be lyophilised and the resultant lyophilisates used, for example, for the preparation of injection preparations. The compositions indicated may be sterilised and/or comprise adjuvants, such as lubricants, preservatives, stabilisers and/or wetting agents, emulsifiers, salts for modifying the osmotic pressure, buffer substances, dyes, flavours and/or one or more further active ingredients, for example one or more vitamins.

In general, the substances according to the invention are preferably administered here in doses of between 1 and 500 mg, in particular between 5 and 100 mg, per dosage unit. The daily dose is preferably between about 0.02 and 10 mg/kg of body weight. However, the specific dose for each patient depends on a very wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

Preferred compounds of the formula I have nanomolar affinity to the 5 HT2A receptors, with in some case low affinity to the 5 HT2C receptor.

Above and below, all temperatures are indicated in ° C. In the following examples, “conventional work-up” means: water is added if necessary, the mixture is extracted with ethyl acetate or dichloromethane, the phases are separated, the organic phase is dried over sodium sulfate and evaporated, and the product is purified by chromatography on silica gel and/or by crystallisation.

### EXAMPLE 1

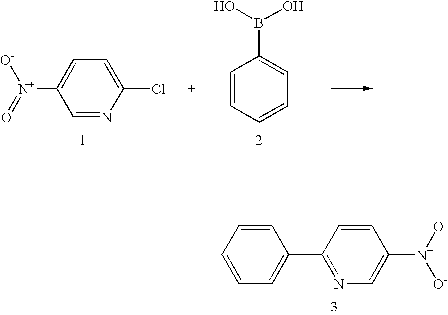

A solution of 6.218 g of 1 and 1.360 g of tetrakis(triphenylphosphine)palladium(0) in 200 ml of ethylene glycol dimethyl ether is gently warmed and, after addition of 5.26 g of 2 and 13.107 g of caesium fluoride, heated under reflux for 6 hrs. Conventional work-up of the reaction mixture gives 3.

### EXAMPLE 2

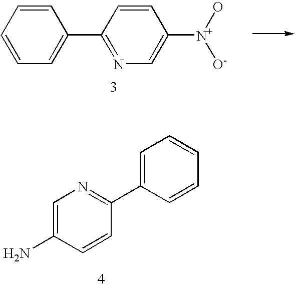

3.02 g of 3 are hydrogenated at atmospheric pressure in the presence of 1.50 g of Raney nickel in 160 ml of methanol. Conventional work-up gives 4.

### EXAMPLE 3

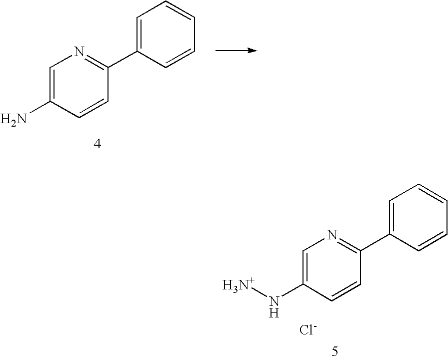

2.34 g of 4 are added to 23.3 ml of water, and 43.1 ml of 32% aqueous hydrochloric acid are added dropwise over the course of 15 min. with stirring at −5° C. to 0° C. A solution of 0.949 g of sodium nitrite in 11.4 ml of water is subsequently added dropwise over the course of 20 min. stirred for a further 30 min. The resultant mixture is added dropwise over the course of 20 min. at −5° C. to 0° C. to a solution of 15.58 g of tin(II) chloride dihydrate and 35.3 ml of concentrated hydrochloric acid. The solvent is removed, and the residue is subjected to conventional work-up, giving 5.

### EXAMPLE 4

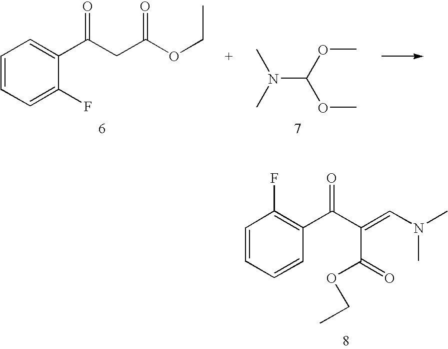

A solution of 41.00 ml of 6 and 61.97 ml of 7 in 820 ml of tetrahydrofuran is stirred for 80 hours and subsequently distilled, giving 8 (b.p. 161° C. at 0.4 mbar).

### EXAMPLE 5

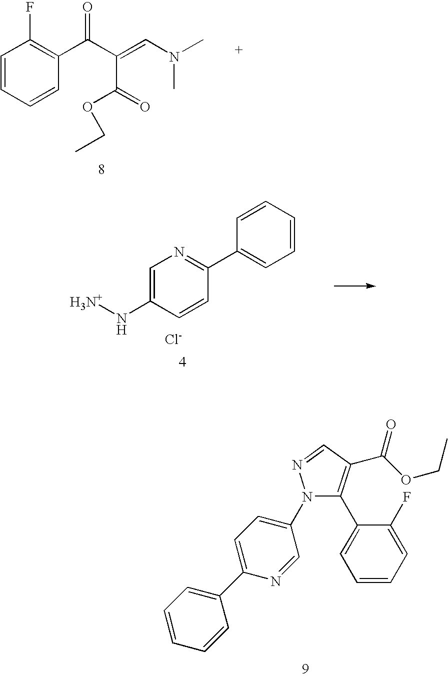

3.95 g of 8, 3.30 g of 4 and 170 ml of ethanol are combined and heated under reflux for 5 hours. Conventional work-up of the reaction mixture gives 9.

### EXAMPLE 6

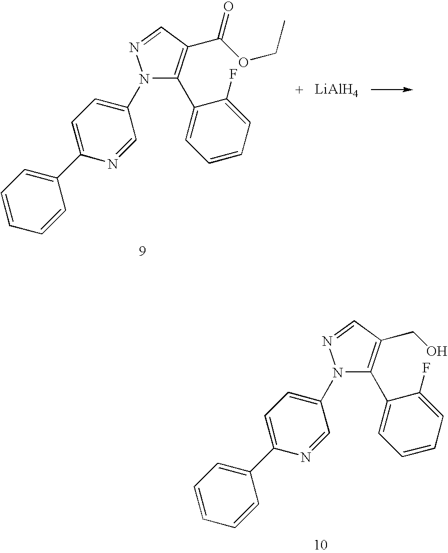

A solution of 2.090 g of 9 in 25 ml of THF is added dropwise with stirring and ice-cooling under a nitrogen atmosphere to a suspension of 1.139 g of lithium aluminium hydride in 25 ml of tetrahydrofuran. After stirring for 1 h, a further 0.500 g of lithium aluminium hydride are added. After stirring for a further 2 h, saturated sodium chloride solution is added dropwise with ice-cooling, and the mixture is subjected to conventional work-up, giving 10.

### EXAMPLE 7

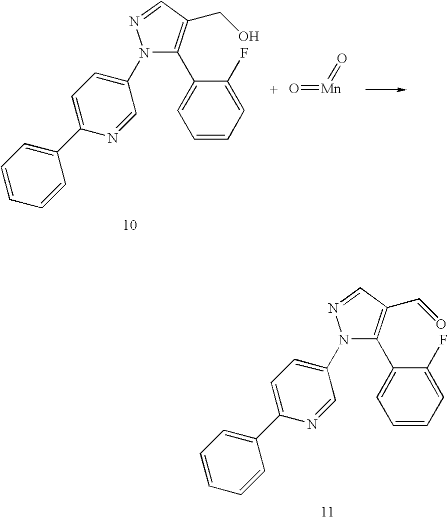

1.480 g of 10, 2.897 g of manganese(IV) oxide, 9.00 ml of tetrahydrofuran and 3.0 ml of dichloromethane are combined and stirred for 3 days. After filtration, the solvent is removed, and the residue is subjected to conventional work-up, giving 11.

### EXAMPLE 8

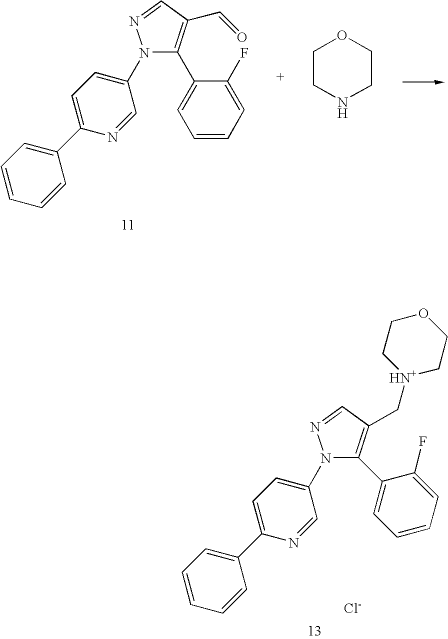

0.017 ml of acetic acid is added to a solution of 0.103 g of 11 and 0.040 ml of 12 in 2.00 ml of dichloroethane and 1.00 ml of tetrahydrofuran, and the mixture is stirred for 3 hours. After addition of 0.120 g of sodium triacetoxyborohydride, the mixture is stirred overnight, saturated sodium hydrogencarbonate is subsequently added, and the mixture is subjected to conventional work-up, giving 13.

### EXAMPLE 9

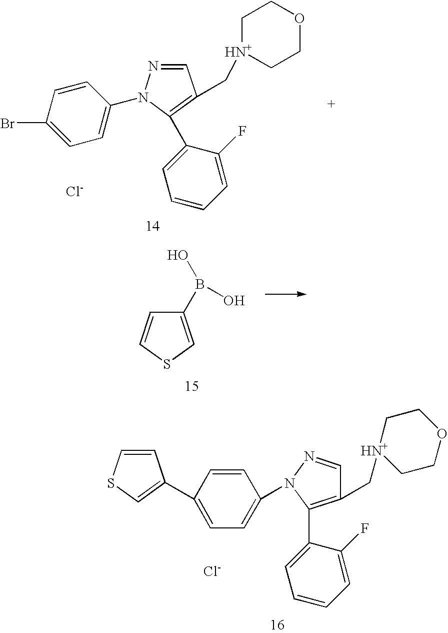

1.00 ml of a 2M sodium carbonate solution is added dropwise to a solution of 91.30 mg of 14; 46.00 mg of 15 and 6.500 mg of bisdichloropalladium(II) in 3.00 ml of dimethoxyethane. The mixture is heated under reflux overnight. After cooling, 5 ml of water are added to the batch, which is subjected to conventional work-up, giving 16.

### EXAMPLE 10

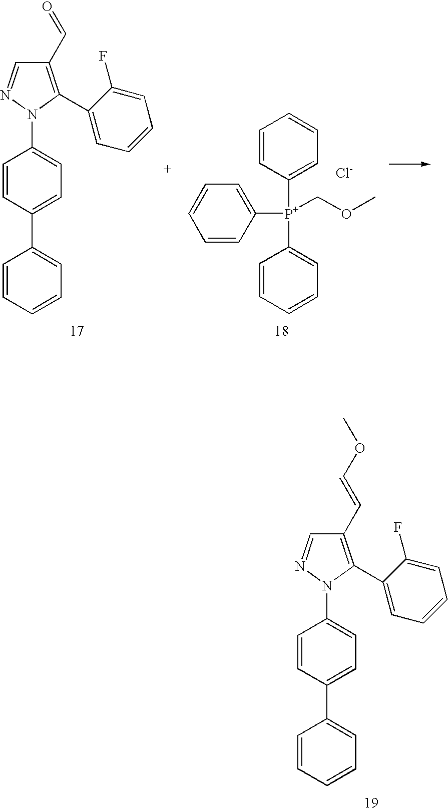

A solution of 0.258 g of potassium tert-butoxide in 5 ml of THF is added dropwise at a max. of 7° C. with stirring and ice-cooling to a solution of 0.685 g of 17 and 0.789 g of 218 in 10 ml of THF. The reaction mixture is stirred for 2 days and subsequently subjected to conventional work-up, giving 19.

### EXAMPLE 11

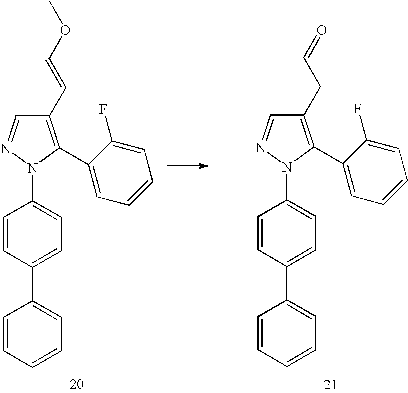

A mixture of 50.00 mg of 20, 3.00 ml of a 16% aqueous sulfuric acid and 3.00 ml of toluene is heated under reflux for 2 hours. The mixture is subsequently left to stir at room temperature for 3 days. Conventional work-up gives 21.

### EXAMPLE 12

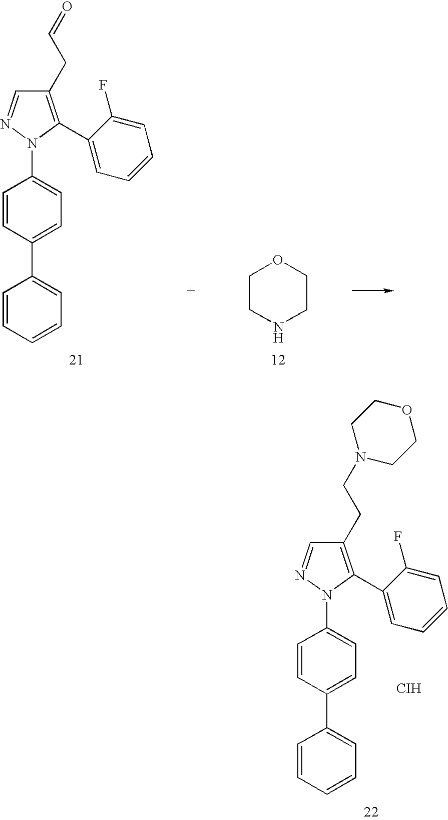

0.010 ml of acetic acid are added to a solution of 61.000 mg of 21 and 22.35 mg of morpholine in 3.000 ml of dichloroethane and 1.5 ml of tetrahydrofuran. The mixture is stirred for 3 h, and 68.668 mg of sodium triacetoxyborohydride are subsequently added. After stirring for 2 days, the mixture is subjected to conventional work-up, giving the free base of 22. After reaction of the base with one equivalent of a 0.1 M HCl/2-propanol solution, the hydrochloride 22 precipitates out by addition of methyl tert-butyl ether, enabling it to be isolated by filtration.

### EXAMPLE 13

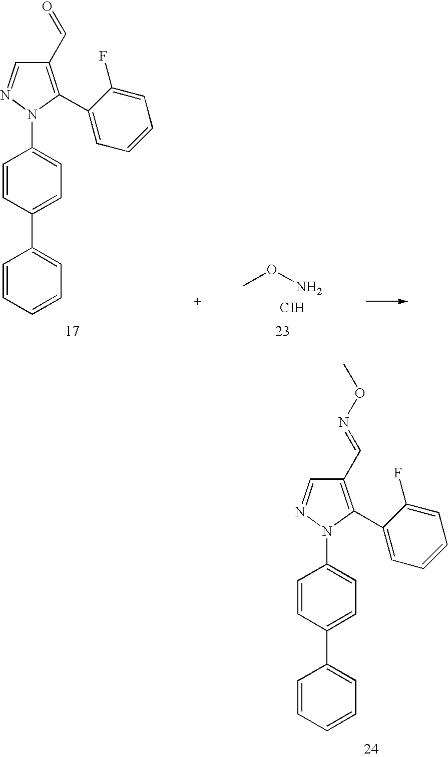

0.033 ml of acetic acid are added to a solution of 200.00 mg of 17 and 74.66 mg of o-methylhydroxylamine hydrochloride 23 in 8.50 ml of dichloroethane and 4.5 ml of tetrahydrofuran, and the mixture is stirred for 3 h. The mixture is stirred for 3 h, and 130.287 mg of sodium triacetoxyborohydride are subsequently added. After stirring for 5 hours, the mixture is subjected to conventional work-up, giving 24.

### EXAMPLE 14

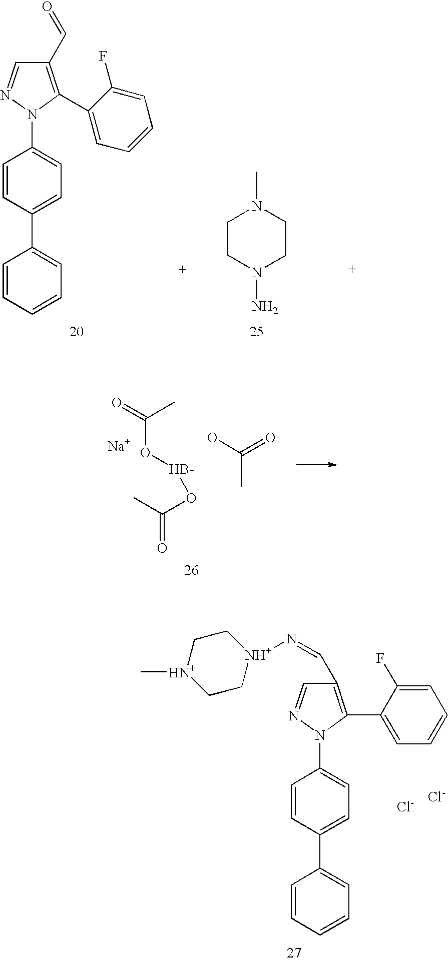

0.026 ml of acetic acid is added to 0.160 g of 17 and 0.087 ml of 25 in a mixture of 3.00 ml of dichloroethane and 1.50 ml of tetrahydrofuran, and the mixture is stirred for 3 hours.

After addition of 0.188 g of 26, stirring is continued overnight, and the mixture is subjected to conventional work-up, giving 28, the free base of 27. Reaction with 1 equivalent of a 0.1 M solution of HCl in 2-propanol enables the hydrochloride 27 to be obtained.

### EXAMPLE 15

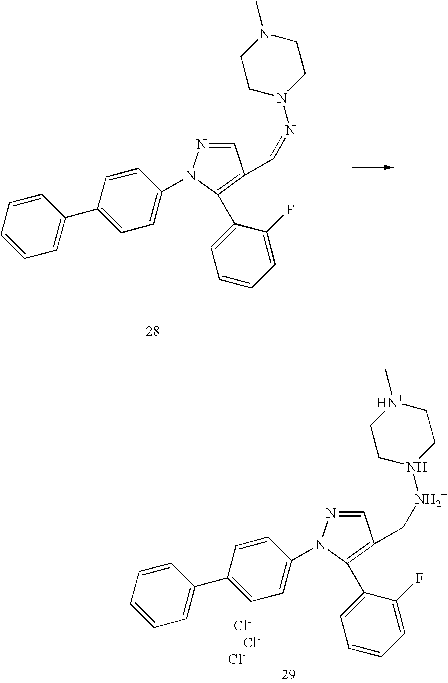

80.00 mg of 28 are hydrogenated at atmospheric pressure in the presence of 0.70 g of Raney nickel in 10 ml of ethanol. Conventional work-up and addition of hydrochloric acid gives 29.

### EXAMPLE 16

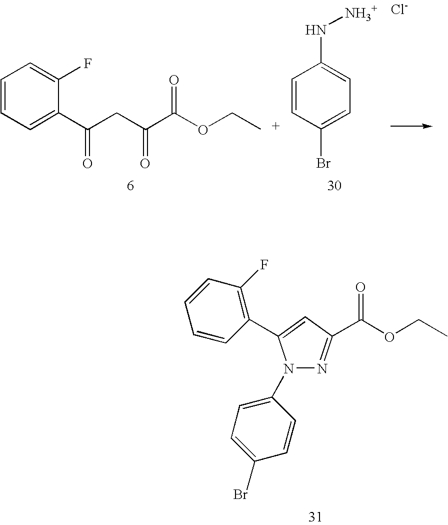

1.20 g of 6, 2.70 g of 30, 6.0 ml of hydrochloric acid and 40.0 ml of dimethylacetamide are combined and stirred overnight. After addition of 40 ml of water, the mixture is stirred for a further 4 h and subjected to conventional work-up, giving 31.

### EXAMPLE 17

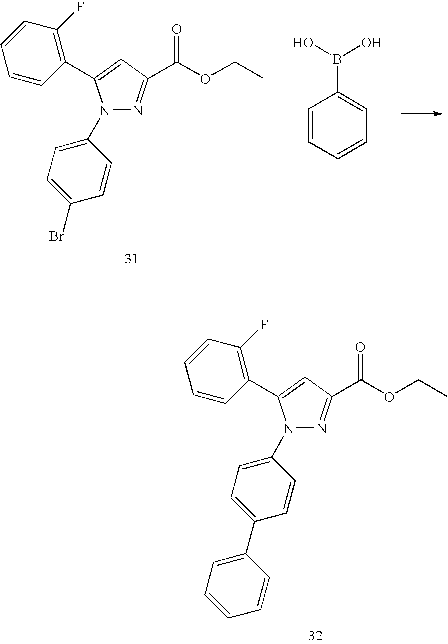

4.00 ml of an aqueous 2M sodium carbonate solution and 150.00 mg of tetrakis(triphenylphosphine)palladium(0) are added to a solution of 1.00 g of 31 and 630.0 mg of 2 in 15.0 ml of ethylene glycol dimethyl ether. The mixture is heated under reflux for 3 hours. After cooling, the mixture is subjected to conventional work-up, giving 32.

### EXAMPLE 18

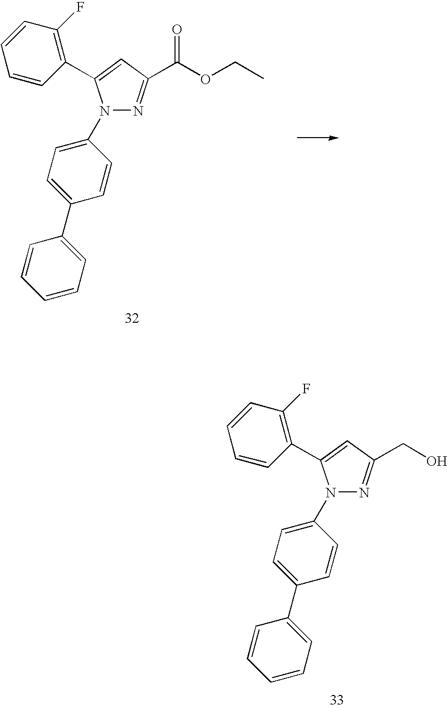

A solution of 3.6 g of 32 in 30 ml of tetrahydrofuran is added dropwise in a nitrogen atmosphere to a suspension of 450.00 mg of lithium aluminium hydride in 20 ml of tetrahydrofuran The mixture is stirred for 2 hours. 50 ml of a mixture of water and tetrahydrofuran (1:1 v/v) are slowly added dropwise with ice-cooling, the resultant precipitate is filtered off with suction, and the filtrate is subjected to conventional work-up, giving 33.

### EXAMPLE 19

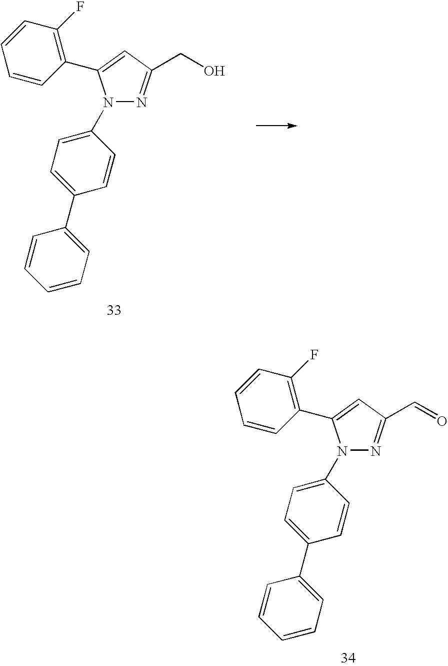

1.600 g of 33, 4.00 g of manganese(IV) oxide and 50.00 ml of dichloromethane are combined and stirred at room temperature at 4 hours. After addition of a further 2 g of manganese(IV) oxide, the mixture is stirred for 2 days and subsequently subjected to conventional work-up, giving 34.

### EXAMPLE 20

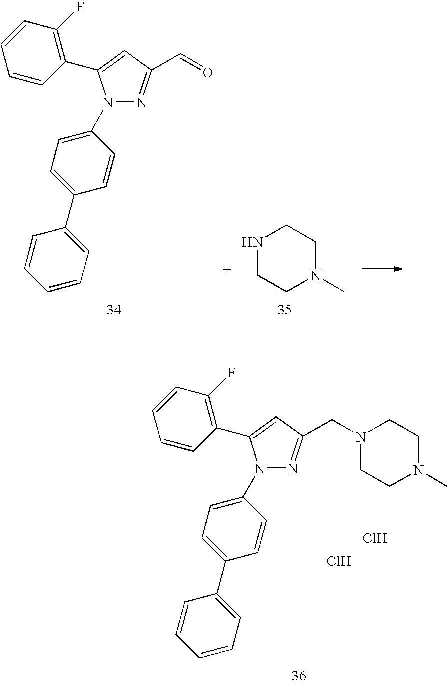

0.10 ml of acetic acid is added to a solution of 430.00 mg of 34 and 0.210 ml of 35 in 10.0 ml of dichloroethane and 5.0 ml of tetrahydrofuran. The reaction mixture is stirred for 3 hours. 0.50 g of sodium triacetoborohydride are subsequently added, the mixture is stirred for 2 hours and then subjected to conventional work-up, giving the free base of 36, from which 36 is obtained in crystalline form (m.p.:277° C.) by addition of ethereal HCl.

The following compounds are obtained analogously for the use according to the invention using the corresponding precursors:

### EXAMPLES 21-240

| | | IC50 [mol/l] |
|---|---|---|
| (21) | [1-Biphenyl-4-yl-5-(2-fluorophenyl)-1H-pyrazol-4-yl]- | 1.20E−06 |
| | methanol | |
| (22) | 1-Biphenyl-4-yl-5-(2-fluorophenyl)-1H-pyrazol-4-ylmethyl | 1.40E−06 |
| | acetate | |
| (23) | 1-[1-Biphenyl-4-yl-5-(2-fluorophenyl)-1H-pyrazol-4-ylmethyl]- | 3.00E−08 |
| | piperidine | |
| (24) | 1-Benzyl-4-[1-biphenyl-4-yl-5-(2-fluorophenyl)-1H- | 1.70E−07 |
| | pyrazol-4-ylmethyl]piperazine | |
| (25) | 4-{1-[1-Biphenyl-4-yl-5-(2-fluorophenyl)-1H-pyrazol-4- | 5.60E−07 |
| | ylmethyl]piperidin-4-yl}morpholine | |
| (26) | [1-Biphenyl-4-yl-5-(2-fluorophenyl)-1H-pyrazol-4-ylmethyl]- | 3.40E−08 |
| | (3-methoxypropyl)amine | |
| (27) | 2-[1-Biphenyl-4-yl-5-(2-fluorophenyl)-1H-pyrazol-4-ylmethyl]- | 2.80E−07 |
| | 1,3,4,6,7,11b-hexahydro-2H-pyrazino[2,1-a]- | |
| | isoquinoline | |
| (28) | 4-[1-Biphenyl-4-yl-5-(2-fluorophenyl)-1H-pyrazol-4-ylmethyl]- | 1.10E−06 |
| | morpholine | |
| (29) | {[1-Biphenyl-4-yl-5-(2-fluorophenyl)-1H-pyrazol-4-ylmethyl]- | 1.70E−06 |
| | amino}acetic acid | |
| (30) | 1-Biphenyl-4-yl-4-(2,5-dihydropyrrol-1-ylmethyl)-5-(2- | 3.60E−08 |
| | fluorophenyl)-1H-pyrazole | |
| (31) | 1-[1-Biphenyl-4-yl-5-(2-fluorophenyl)-1H-pyrazol-4-ylmethyl]- | 4.80E−08 |
| | azepan | |
| (32) | Benzyl-[1-biphenyl-4-yl-5-(2-fluorophenyl)-1H-pyrazol- | 3.20E−07 |
| | 4-ylmethyl]ethylamine | |
| (33) | [1-Biphenyl-4-yl-5-(2-fluorophenyl)-1H-pyrazol-4-ylmethyl]- | 5.50E−08 |
| | diethylamine | |
| (34) | [1-Biphenyl-4-yl-5-(2-fluorophenyl)-1H-pyrazol-4-ylmethyl]- | 2.10E−08 |
| | dimethylamine | |
| (35) | 1-Biphenyl-4-yl-5-(2-fluorophenyl)-4-pyrrolidin-1-ylmethyl- | 3.20E−08 |
| | 1H-pyrazole | |
| (36) | 2-[1-Biphenyl-4-yl-5-(2-fluorophenyl)-1H-pyrazol-4-ylmethyl]- | 7.00E−08 |
| | 1,2,3,4-tetrahydroisoquinoline | |
| (37) | {1-[1-Biphenyl-4-yl-5-(2-fluorophenyl)-1H-pyrazol-4-ylmethyl]- | 2.00E−07 |
| | piperidin-4-yl}dimethylamine | |
| (38) | 1-[1-Biphenyl-4-yl-5-(2-fluorophenyl)-1H-pyrazol-4-ylmethyl]- | 1.70E−07 |
| | 1,2,3,6-tetrahydropyridine | |
| (39) | [1-Biphenyl-4-yl-5-(2-fluorophenyl)-1H-pyrazol-4-ylmethyl]- | 1.60E−07 |
| | methyl-(1-methylpiperidin-4-yl)amine | |
| (40) | [1-Biphenyl-4-yl-5-(2-fluorophenyl)-1H-pyrazol-4-ylmethyl]- | 1.40E−08 |
| | (4-methylpiperazin-1-yl)amine | |
| (41) | 1-[1-Biphenyl-4-yl-5-(2-fluorophenyl)-1H-pyrazol-4-ylmethyl]- | 2.40E−08 |
| | 4-methylpiperazine | |
| (42) | 4-{2-[1-Biphenyl-4-yl-5-(2-fluorophenyl)-1H-pyrazol-4- | 1.20E−08 |
| | yl]ethyl}morpholine | |
| (43) | 1-[1-Biphenyl-4-yl-5-(2-fluorophenyl)-1H-pyrazol-3-ylmethyl]- | 1.20E−07 |
| | piperidine | |
| (44) | 4-[1-Biphenyl-4-yl-5-(2-fluorophenyl)-1H-pyrazol-3-ylmethyl]- | 1.10E−06 |
| | morpholine | |
| (45) | 1-[1-Biphenyl-4-yl-5-(2-fluorophenyl)-1H-pyrazol-3-ylmethyl]- | 3.00E−07 |
| | 4-methylpiperazine | |
| (46) | 4-{3-[1-Biphenyl-4-yl-5-(2-fluorophenyl)-1H-pyrazol-4- | 1.70E−08 |
| | yl]allyl}morpholine | |
| (47) | 4-{3-[1-Biphenyl-4-yl-5-(2-fluorophenyl)-1H-pyrazol-4- | 2.30E−08 |
| | yl]propyl}morpholine | |
| (48) | 1-Biphenyl-4-yl-5-(2-fluorophenyl)-4-(2-methoxy- | 5.10E−07 |
| | methylpyrrolidin-1-ylmethyl)-1H-pyrazole | |
| (49) | 1-[1-Biphenyl-4-yl-5-(2-fluorophenyl)-1H-pyrazol-4-ylmethyl]- | 1.30E−07 |
| | 4-methylpiperidine | |
| (50) | N-{1-[1-Biphenyl-4-yl-5-(2-fluorophenyl)-1H-pyrazol-4- | 2.90E−08 |
| | ylmethyl]pyrrolidin-3-yl}acetamide | |
| (51) | {1-[1-Biphenyl-4-yl-5-(2-fluorophenyl)-1H-pyrazol-4-ylmethyl]- | 2.70E−07 |
| | piperidin-2-ylmethyl}diethylamine | |
| (52) | 1-[1-Biphenyl-4-yl-5-(2-fluorophenyl)-1H-pyrazol-4-yl]- | 8.20E−07 |
| | 1-(4-methylpiperazin-1-yl)methanone | |
| (53) | 1-[1-Biphenyl-4-yl-5-(2-fluorophenyl)-1H-pyrazol-4-ylmethyl]- | 1.40E−08 |
| | pyrrolidin-3-ol | |
| (54) | tert-Butyl 4-[1-Biphenyl-4-yl-5-(2-fluorophenyl)-1H- | 8.20E−08 |
| | pyrazol-4-ylmethyl]piperazine-1-carboxylate | |
| (55) | 1-[1-Biphenyl-4-yl-5-(2-fluorophenyl)-1H-pyrazol-4-ylmethyl]- | 2.60E−08 |
| | piperazine | |
| (56) | 1-[5-(2-Fluorophenyl)-1-(6-phenylpyridin-3-yl)-1H-pyrazol- | 1.50E−07 |
| | 4-ylmethyl]piperazine | |
| (57) | 1-[5-(2-Fluorophenyl)-1-(6-phenylpyridin-3-yl)-1H-pyrazol- | 2.20E−07 |
| | 4-ylmethyl]-4-methylpiperazine | |
| (58) | 1-(1-Biphenyl-4-yl-5-pyridin-2-yl-1H-pyrazol-4-ylmethyl)- | 5.00E−08 |
| | 4-methylpiperazine | |
| (59) | tert-Butyl 4-(1-biphenyl-4-yl-5-pyridin-2-yl-1H-pyrazol- | 7.80E−07 |
| | 4-ylmethyl)piperazine-1-carboxylate | |
| (60) | 1-(1-Biphenyl-4-yl-5-pyridin-2-yl-1H-pyrazol-4-ylmethyl)- | 2.00E−07 |
| | piperazine | |
| (61) | 4-[1-(4′-Fluorobiphenyl-4-yl)-5-(2-fluorophenyl)-1H- | 5.20E−07 |
| | pyrazol-4-ylmethyl]morpholine | |
| (62) | 4-[5-(2-Fluorophenyl)-1-(4-thiophen-2-ylphenyl)-1H- | 6.20E−07 |
| | pyrazol-4-ylmethyl]morpholine | |
| (63) | 1-[1-(2′-Fluorobiphenyl-4-yl)-5-(2-fluorophenyl)-1H- | 9.30E−08 |
| | pyrazol-4-ylmethyl]-4-methylpiperazine | |
| (64) | 1-[1-(4′-Fluorobiphenyl-4-yl)-5-(2-fluorophenyl)-1H- | 7.80E−09 |
| | pyrazol-4-ylmethyl]-4-methylpiperazine | |
| (65) | 1-[1-(2′,5′-Difluorobiphenyl-4-yl)-5-(2-fluorophenyl)-1H- | 5.50E−08 |
| | pyrazol-4-ylmethyl]-4-methylpiperazine | |
| (66) | 1-[5-(2-Fluorophenyl)-1-(4-thiophen-3-ylphenyl)-1H- | 1.40E−08 |
| | pyrazol-4-ylmethyl]-4-methylpiperazine | |
| (67) | 1-(Biphenyl-4-yltrifluoromethyl-1H-pyrazol-4-ylmethyl)- | 2.60E−07 |
| | 4-methylpiperazine | |
| (68) | 1-[1-(2′,5′-Difluorobiphenyl-4-yl)-5-phenyl-1H-pyrazol-4- | 1.10E−07 |
| | ylmethyl]-4-methylpiperazine | |
| (69) | 1-[1-(2′,5′-Difluorobiphenyl-4-yl)-5-furan-2-yl-1H-pyrazol- | 3.60E−08 |
| | 4-ylmethyl]-4-methylpiperazine | |
| (70) | 1-[5-Furan-2-yl-1-(4-thiophen-3-ylphenyl)-1H-pyrazol-4- | 4.50E−09 |
| | ylmethyl]-4-methylpiperazine | |
| (71) | 1-[1-(2′,5′-Difluorobiphenyl-4-yl)-5-(2-methoxyphenyl)- | 7.70E−08 |
| | 1H-pyrazol-4-ylmethyl]-4-methylpiperazine | |
| (72) | Ethyl 4-{[1-biphenyl-4-yl-5-(2-fluorophenyl)-1H-pyrazol- | 5.80E−07 |
| | 4-ylmethyl]amino}piperidine-1-carboxylate | |
| (73) | Ethyl {4-[1-biphenyl-4-yl-5-(2-fluorophenyl)-1H-pyrazol- | 6.90E−07 |
| | 4-ylmethyl]piperazin-1-yl}acetate | |
| (74) | [1-Biphenyl-4-yl-5-(2-fluorophenyl)-1H-pyrazol-4-ylmethyl]- | 4.70E−07 |
| | piperidin-4-ylamine | |
| (75) | {4-[1-Biphenyl-4-yl-5-(2-fluorophenyl)-1H-pyrazol-4-ylmethyl]- | 6.30E−07 |
| | piperazin-1-yl}- | |
| (76) | N¹-[1-Biphenyl-4-yl-5-(2-fluorophenyl)-1H-pyrazol-4-ylmethyl]- | 6.50E−09 |
| | ethane-1,2-diamine | |
| (77) | 2-{[1-Biphenyl-4-yl-5-(2-fluorophenyl)-1H-pyrazol-4-ylmethyl]- | 5.20E−09 |
| | amino}ethanol | |
| (78) | [1-Biphenyl-4-yl-5-(2-fluorophenyl)-1H-pyrazol-4-ylmethyl]- | 1.60E−08 |
| | (2-methoxyethyl)amine | |
| (79) | 2-{4-[1-Biphenyl-4-yl-5-(2-fluorophenyl)-1H-pyrazol-4- | 2.80E−07 |
| | ylmethyl]piperazin-1-yl}ethanol | |
| (80) | 1-[1-Biphenyl-4-yl-5-(2-fluorophenyl)-1H-pyrazol-4-ylmethyl]- | 2.80E−07 |
| | 4-ethylpiperidin-4-ol | |
| (81) | 1-[1-Biphenyl-4-yl-5-(2-fluorophenyl)-1H-pyrazol-4-ylmethyl]- | 4.30E−07 |
| | piperidin-4-ol | |
| (82) | 5-[1-Biphenyl-4-yl-5-(2-fluorophenyl)-1H-pyrazol-4-ylmethyl]- | 1.60E−07 |
| | 2-oxa-5-azabicyclo[2.2.1]heptane | |
| (83) | 8-[1-Biphenyl-4-yl-5-(2-fluorophenyl)-1H-pyrazol-4-ylmethyl]- | 1.10E−06 |
| | 8-azabicyclo[3.2.1]octan-3-ol | |
| (84) | tert-Butyl 4-[5-(2-fluorophenyl)-1-(4-trifluoromethyl- | 8.00E−09 |
| | phenyl)-1H-pyrazol-4-ylmethyl]piperazine-1-carboxylate | |
| (85) | 1-[1-Biphenyl-4-yl-5-(2-fluorophenyl)-1H-pyrazol-4-ylmethyl]- | 8.70E−07 |
| | piperidine-4-carboxamide | |
| (86) | 1-[5-(2-Fluorophenyl)-1-(4-trifluoromethylphenyl)-1H- | 4.30E−08 |
| | pyrazol-4-ylmethyl]piperazine | |
| (87) | 1-[5-(2-Fluorophenyl)-1-(4-trifluoromethylphenyl)-1H- | 1.60E−07 |
| | pyrazol-4-ylmethyl]-4-methylpiperazine | |
| (88) | [1-Biphenyl-4-yl-5-(2-fluorophenyl)-1H-pyrazol-4-ylmethyl]- | 2.00E−08 |
| | (1-ethylpyrrolidin-2-ylmethyl)amine | |
| (89) | [1-Biphenyl-4-yl-5-(2-fluorophenyl)-1H-pyrazol-4-ylmethyl]- | 1.80E−07 |
| | pyridin-3-ylmethylamine | |
| (90) | 1-[1-Biphenyl-4-yl-5-(2-fluorophenyl)-1H-pyrazol-4-ylmethyl]- | 2.80E−08 |
| | 4-ethylpiperazine | |
| (91) | 2-{4-[1-Biphenyl-4-yl-5-(2-fluorophenyl)-1H-pyrazol-4- | 3.70E−08 |
| | ylmethyl]piperazin-1-yl}-1-pyrrolidin-1-ylethanone | |
| (92) | 2-{[1-Biphenyl-4-yl-5-(2-fluorophenyl)-1H-pyrazol-4-ylmethyl]- | 1.60E−08 |
| | methylamino}ethanol | |
| (93) | 1-[1-Biphenyl-4-yl-5-(2-fluorophenyl)-1H-pyrazol-4-ylmethyl]- | 6.40E−09 |
| | 4-methyl-[1,4]diazepam | |
| (94) | 8-[1-Biphenyl-4-yl-5-(2-fluorophenyl)-1H-pyrazol-4-ylmethyl]- | 4.00E−07 |
| | 1-phenyl-1,3,8-triazaspiro[4.5]decan-4-one | |
| (95) | 1-[1-Biphenyl-4-yl-5-(2-fluorophenyl)-1H-pyrazol-4-ylmethyl]- | 1.00E−07 |
| | 3,5-dimethylpiperazine | |
| (96) | 1-[1-(2′-Fluorobiphenyl-4-yl)-5-phenyl-1H-pyrazol-4-ylmethyl]- | 8.20E−07 |
| | 4-methylpiperazine | |
| (97) | 1-[1-(4′-Fluorobiphenyl-4-yl)-5-phenyl-1H-pyrazol-4-ylmethyl]- | 1.30E−08 |
| | 4-methylpiperazine | |
| (98) | 1-Methyl-4-[5-phenyl-1-(4-thiophen-3-ylphenyl)-1H- | 3.10E−08 |
| | pyrazol-4-ylmethyl]piperazine | |
| (99) | 1-[1-(2′-Fluorobiphenyl-4-yl)-5-(4-fluorophenyl)-1H- | 2.10E−07 |
| | pyrazol-4-ylmethyl]-4-methylpiperazine | |
| (100) | 1-[1-(4′-Fluorobiphenyl-4-yl)-5-(4-fluorophenyl)-1H- | 2.50E−08 |
| | pyrazol-4-ylmethyl]-4-methylpiperazine | |
| (101) | 1-[1-(2′,5′-Difluorobiphenyl-4-yl)-5-(4-fluorophenyl)-1H- | 2.40E−07 |
| | pyrazol-4-ylmethyl]-4-methylpiperazine | |
| (102) | 1-[5-(4-Fluorophenyl)-1-(4-thiophen-3-ylphenyl)-1H- | 1.10E−06 |
| | pyrazol-4-ylmethyl]-4-methylpiperazine | |
| (103) | 1-[1-(2′-Fluorobiphenyl-4-yl)-5-furan-2-yl-1H-pyrazol-4- | 2.50E−08 |
| | ylmethyl]-4-methylpiperazine | |
| (104) | 1-[1-(2′-Fluorobiphenyl-4-yl)-5-(2-methoxyphenyl)-1H- | 5.20E−08 |
| | pyrazol-4-ylmethyl]-4-methylpiperazine | |
| (105) | 1-[5-(2-Methoxyphenyl)-1-(4-thiophen-3-ylphenyl)-1H- | 4.60E−07 |
| | pyrazol-4-ylmethyl]-4-methylpiperazine | |
| (106) | 1-[5-(2-Fluorophenyl)-1-(4-pyrrol-1-ylphenyl)-1H-pyrazol- | 1.70E−08 |
| | 4-ylmethyl]-4-methylpiperazine | |
| (107) | (1-Azabicyclo[2.2.2]oct-3-yl)-[1-biphenyl-4-yl-5-(2- | 1.40E−07 |
| | fluorophenyl)-1H-pyrazol-4-ylmethyl]amine | |
| (108) | 4-[1-Biphenyl-4-yl-5-(2-fluorophenyl)-1H-pyrazol-4-ylmethyl]- | 4.30E−08 |
| | thiomorpholine 1,1-dioxide | |
| (109) | 2-[1-Biphenyl-4-yl-5-(2-fluorophenyl)-1H-pyrazol-4-ylmethyl]- | 9.40E−08 |
| | 5-methyl-2,5-diazabicyclo[2.2.1]heptane | |
| (110) | Ethyl 4-{[1-biphenyl-4-yl-5-(2-fluorophenyl)-1H-pyrazol- | 1.70E−07 |
| | 4-ylmethyl]methylamino}piperidine-1-carboxylate | |
| (111) | 2-{4-[1-Biphenyl-4-yl-5-(2-fluorophenyl)-1H-pyrazol-4- | 3.60E−07 |
| | ylmethyl]piperazin-1-yl}acetamide | |
| (112) | Methyl 3-{[1-biphenyl-4-yl-5-(2-fluorophenyl)-1H-pyrazol- | 2.10E−07 |
| | 4-ylmethyl]amino}propionate | |
| (113) | 1-(1-Biphenyl-4-yl-5-pyridin-3-yl-1H-pyrazol-4-ylmethyl)- | 2.90E−07 |
| | 4-methylpiperazine | |
| (114) | 1-[5-(2-Fluorophenyl)-1-(4-isopropylphenyl)-1H-pyrazol- | 1.20E−07 |
| | 4-ylmethyl]-4-methylpiperazine | |
| (115) | 1-[5-(2-Fluorophenyl)-1-(4-trifluoromethoxyphenyl)-1H- | 2.70E−07 |
| | pyrazol-4-ylmethyl]-4-methylpiperazine | |
| (116) | 1-{5-(2-Fluorophenyl)-1-[6-(4-fluorophenyl)pyridin-3-yl]- | 1.20E−07 |
| | 1H-pyrazol-4-ylmethyl}-4-methylpiperazine | |
| (117) | Ethyl [(1-biphenyl-4-yl-5-pyridin-3-yl-1H-pyrazol-4-ylmethyl)- | 9.40E−07 |
| | amino]acetate | |
| (118) | 2-{[1-Biphenyl-4-yl-5-(2-fluorophenyl)-1H-pyrazol-4-ylmethyl]- | 7.30E−07 |
| | amino}acetamide | |
| (119) | {5-(2-Fluorophenyl)-1-[6-(4-fluorophenyl)pyridin-3-yl]- | 3.00E−07 |
| | 1H-pyrazol-4-yl}methanol | |
| (120) | 1-[5-(2-Fluorophenyl)-1-(4-pyridin-3-ylphenyl)-1H-pyrazol- | 1.70E−07 |
| | 4-ylmethyl]-4-methylpiperazine | |
| (121) | 1-[5-(2-Fluorophenyl)-1-(4-pyridin-4-ylphenyl)-1H-pyrazol- | 7.70E−08 |
| | 4-ylmethyl]-4-methylpiperazine | |
| (122) | [1-(2′,5′-Difluorobiphenyl-4-yl)-5-(2-fluorophenyl)-1H- | 3.70E−07 |
| | pyrazol-4-ylmethyl](tetrahydrofuran-2-ylmethyl)amine | |
| (123) | 1-[1-Biphenyl-4-yl-5-(2-fluorophenyl)-1H-pyrazol-4-ylmethyl]- | 8.30E−07 |
| | 4-methanesulfonylpiperazine | |
| (124) | {[1-Biphenyl-4-yl-5-(2-fluorophenyl)-1H-pyrazol-4-ylmethyl]- | 2.80E−07 |
| | amino}acetonitrile | |
| (125) | 2-{4-[1-Biphenyl-4-yl-5-(2-fluorophenyl)-1H-pyrazol-4- | 5.10E−07 |
| | ylmethyl]piperazin-1-yl}-1-pyrrolidin-1-ylethanone | |
| (126) | N-Phenyl-4-[1-biphenyl-4-yl-5-(2-fluorophenyl)-1H- | 7.90E−07 |
| | pyrazol-4-ylmethyl]piperazine-1-carboxamide | |
| (127) | N-[5-(2-Fluorophenyl)-1-(6-phenylpyridin-3-yl)-1H- | 4.40E−07 |
| | pyrazol-4-ylmethyl]-N,N′,N′-trimethylethane-1,2-diamine | |
| (128) | 2-{[1-Biphenyl-4-yl-5-(2-fluorophenyl)-1H-pyrazol-4-ylmethyl]- | 2.00E−07 |
| | amino}-N,N-dimethylacetamide | |
| (129) | 2-{[1-Biphenyl-4-yl-5-(2-fluorophenyl)-1H-pyrazol-4-ylmethyl]- | 1.00E−07 |
| | amino}-N-(4-nitrophenyl)acetamide | |
| (130) | 2-{[1-Biphenyl-4-yl-5-(2-fluorophenyl)-1H-pyrazol-4-ylmethyl]- | 6.00E−07 |
| | amino}-N-methylacetamide | |
| (131) | 4-{[1-Biphenyl-4-yl-5-(2-fluorophenyl)-1H-pyrazol-4-ylmethyl]- | 6.00E−06 |
| | amino}isoxazolidin-3-one | |
| (132) | 2-{[1-Biphenyl-4-yl-5-(2-fluorophenyl)-1H-pyrazol-4-ylmethyl]- | 9.20E−07 |
| | methylamino}acetamide | |
| (133) | (1H-Benzoimidazol-2-ylmethyl)-[1-biphenyl-4-yl-5-(2- | 7.60E−08 |
| | fluorophenyl)-1H-pyrazol-4-ylmethyl]amine | |
| (134) | [5-(2-Fluorophenyl)-1-(6-phenylpyridin-3-yl)-1H-pyrazol- | 3.00E−07 |
| | 4-ylmethyl]-(2-methoxyethyl)methylamine | |
| (135) | 1-[1-Biphenyl-4-yl-5-(2-florophenyl)-1H-pyrazol-4-ylmethyl]- | 3.80E−07 |
| | 4-thiophen-3-ylmethylpiperazine | |
| (136) | 2-{[1-Biphenyl-4-yl-5-(2-fluorophenyl)-1H-pyrazol-4-ylmethyl]- | 1.30E−07 |
| | amino}-2-cyanoacetamide | |
| (137) | 2-{[1-Biphenyl-4-yl-5-(2-fluorophenyl)-1H-pyrazol-4-ylmethyl]- | 3.70E−07 |
| | amino}-3-(3H-imidazol-4-yl)propan-1-ol | |
| (138) | [1-Biphenyl-4-yl-5-(2-fluorophenyl)-1H-pyrazol-4-ylmethyl]- | 2.40E−07 |
| | isoxazol-3-ylamine | |
| (139) | 2-{4-[1-Biphenyl-4-yl-5-(2-fluorophenyl)-1H-pyrazol-4- | 2.30E−07 |
| | ylmethyl]piperazin-1-yl}-N-ethylacetamide | |
| (140) | [1-(2′,5′-Difluorobiphenyl-4-yl)-5-(2-fluorophenyl)-1H- | 1.80E−07 |
| | pyrazol-4-ylmethyl]-(1H-pyrazol-3-yl)amine | |
| (141) | N-{5-(2-fluorophenyl)-1-[6-(4-fluorophenyl)pyridin-3-yl]- | 1.40E−07 |
| | 1H-pyrazol-4-ylmethyl}-N,N′,N′-trimethylethane-1,2- | |
| | diamine | |
| (142) | 2-(4-Fluorophenyl)-5-[5-(2-fluorophenyl)-4-pyrrolidin-1- | 7.50E−08 |
| | ylmethylpyrazol-1-yl]pyridine | |
| (143) | {5-(2-Fluorophenyl)-1-[6-(4-fluorophenyl)pyridin-3-yl]- | 2.50E−07 |
| | 1H-pyrazol-4-ylmethyl}methyl-(1-methylpiperidin-4- | |
| | yl)amine | |
| (144) | {5-(2-Fluorophenyl)-1-[6-(4-fluorophenyl)pyridin-3-yl]- | 8.90E−07 |
| | 1H-pyrazol-4-ylmethyl}pyridin-3-ylamine | |
| (145) | 1-{5-(2-Fluorophenyl)-1-[6-(2-fluorophenyl)pyridin-3-yl]- | 2.20E−07 |
| | 1H-pyrazol-4-ylmethyl}pyrrolidine-2-carboxamide | |
| (146) | 4-{5-(2-Fluorophenyl)-1-[6-(2-fluorophenyl)pyridin-3-yl]- | 6.00E−07 |
| | 1H-pyrazol-4-ylmethyl}morpholine | |
| (147) | 1-[1-[6-(2,5-Difluorophenyl)pyridin-3-yl]-5-(2-fluorophenyl)- | 4.30E−07 |
| | 1H-pyrazol-4-ylmethyl]-4-methylpiperazine | |
| (148) | Ethyl ({5-(2-fluorophenyl)-1-[6-(2-fluorophenyl)pyridin- | 1.60E−06 |
| | 3-yl]-1H-pyrazol-4-ylmethyl}amino)acetate | |
| (149) | 1-[1-Biphenyl-4-yl-5-(2-fluorophenyl)-1H-pyrazol-4-yl]- | 6.20E−07 |
| | 4-(4-methylpiperazin-1-yl)butane-1,3-diol | |
| (150) | 1-[1-Biphenyl-4-yl-5-(2-fluorophenyl)-1H-pyrazol-4-yl]- | 1.30E−06 |
| | but-3-en-1-ol | |
| (151) | 1-(3-{[1-Biphenyl-4-yl-5-(2-fluorophenyl)-1H-pyrazol-4- | 6.40E−08 |
| | ylmethyl]amino}propyl)pyrrolidin-2-one | |
| (152) | [1-Biphenyl-4-yl-5-(2-fluorophenyl)-1H-pyrazol-4-ylmethyl]- | 1.30E−07 |
| | (3-imidazol-1-ylpropyl)amine | |
| (153) | Ethyl (2-{[1-Biphenyl-4-yl-5-(2-fluorophenyl)-1H-pyrazol- | 1.10E−06 |
| | 4-ylmethyl]amino}ethanoylamino)acetate | |
| (154) | [1-Biphenyl-4-yl-5-(2-fluorophenyl)-1H-pyrazol-4-ylmethyl]- | 1.70E−07 |
| | [2-(1H-imidazol-4-yl)ethyl]amine | |
| (155) | 1-[1-Biphenyl-4-yl-5-(2-fluorophenyl)-1H-pyrazol-4-ylmethyl]- | 1.70E−06 |
| | pyrrolidine-2-carboxamide | |
| (156) | {5-(2-Fluorophenyl)-1-[6-(4-fluorophenyl)pyridin-3-yl]- | 3.00E−07 |
| | 1H-pyrazol-4-ylmethyl}-(2-methoxyethyl)methylamine | |
| (157) | {5-(2-Fluorophenyl)-1-[6-(2-fluorophenyl)pyridin-3-yl]- | 2.00E−06 |
| | 1H-pyrazol-4-ylmethyl}-(2-methoxyethyl)methylamine | |
| (158) | [1-Biphenyl-4-yl-5-(2-fluorophenyl)-1H-pyrazol-4-ylmethyl]- | 2.30E−06 |
| | pyrazin-2-ylamine | |
| (159) | [1-[6-(2,5-Difluorophenyl)pyridin-3-yl]-5-(2-fluorophenyl)- | 1.40E−06 |
| | 1H-pyrazol-4-ylmethyl]-(2-methoxyethyl)- | |
| | methylamine | |
| (160) | 4-Azetidin-1-ylmethyl-1-biphenyl-4-yl-5-(2-fluorophenyl)- | 4.70E−08 |
| | 1H-pyrazole | |
| (161) | (1-Benzylpyrrolidin-3-yl)-[1-biphenyl-4-yl-5-(2-fluorophenyl)- | 3.10E−07 |
| | 1H-pyrazol-4-ylmethyl]amine | |
| (162) | Methyl 4-{[1-biphenyl-4-yl-5-(2-fluorophenyl)-1H-pyrazol- | 1.20E−07 |
| | 4-ylmethyl]amino}-1-methyl-1H-pyrrole-2-carboxylate | |
| (163) | 3-{[1-Biphenyl-4-yl-5-(2-fluorophenyl)-1H-pyrazol-4-ylmethyl]- | 5.10E−07 |
| | amino}azepan-2-one | |
| (164) | N-(2-Hydroxyethyl)-1-[1-biphenyl-4-yl-5-(2-fluorophenyl)- | 2.70E−07 |
| | 1H-pyrazol-4-ylmethyl]piperidine-4-carboxamide | |
| (165) | C-(1-Biphenyl-4-yl-5-phenyl-1H-pyrazol-4-yl)methylamine | 2.10E−08 |
| (166) | N-[1-Biphenyl-4-yl-5-(2-fluorophenyl)-1H-pyrazol-4-yl- | 1.20E−06 |
| | methylene]-N′-(4,5-dihydro-1H-imidazol-2-yl)hydrazine | |
| (167) | {1-[1-Biphenyl-4-yl-5-(2-fluorophenyl)-1H-pyrazol-4-ylmethyl]- | 2.30E−07 |
| | pyrrolidin-3-yl}dimethylamine | |
| (168) | [1-Biphenyl-4-yl-5-(2-fluorophenyl)-1H-pyrazol-4-ylmethyl]- | 1.60E−08 |
| | methyl-(1-methylpyrrolidin-3-yl)amine | |
| (169) | (1-Biphenyl-4-yl-5-phenyl-1H-pyrazol-4-ylmethyl)- | 3.90E−08 |
| | (1-methyl-1H-imidazol-2-ylmethyl)amine | |
| (170) | (1-Biphenyl-4-yl-5-phenyl-1H-pyrazol-4-ylmethyl) | 3.10E−08 |
| | pyridin-4-ylmethylamine | |
| (171) | (1-Biphenyl-4-yl-5-phenyl-1H-pyrazol-4-ylmethyl)- | 2.60E−08 |
| | (1-methyl-1H-pyrrol-2-ylmethyl)amine | |
| (172) | (1-Biphenyl-4-yl-5-phenyl-1H-pyrazol-4-ylmethyl)pyridin- | 2.30E−08 |
| | 2-ylmethylamine | |
| (173) | 2-[1-Biphenyl-4-yl-5-(2-fluorophenyl)-1H-pyrazol-4-yl]- | 4.70E−07 |
| | ethanol | |
| (174) | {5-(2-Fluorophenyl)-1-[6-(4-fluorophenyl)pyridin-3-yl]- | 3.20E−07 |
| | 1H-pyrazol-4-ylmethyl}isoxazol-3-ylamine | |
| (175) | {5-(2-Fluorophenyl)-1-[6-(4-fluorophenyl)pyridin-3-yl]- | 6.30E−07 |
| | 1H-pyrazol-4-ylmethyl}pyridin-3-ylamine | |
| (176) | tert-Butyl 3-{[1-biphenyl-4-yl-5-(2-fluorophenyl)-1H- | 4.60E−07 |
| | pyrazol-4-ylmethyl]amino}pyrrolidine-1-carboxylate | |
| (177) | N³-[1-Biphenyl-4-yl-5-(2-fluorophenyl)-1H-pyrazol-4-ylmethyl]- | 1.80E−07 |
| | pyridine-3,4-diamine | |
| (178) | [1-Biphenyl-4-yl-5-(2-fluorophenyl)-1H-pyrazol-4-ylmethyl]- | 3.70E−07 |
| | (5-methylthiazol-2-yl)amine | |
| (179) | tert-Butyl [(1-biphenyl-4-yl-5-pyridin-3-yl-1H-pyrazol-4- | 1.00E−06 |
| | ylmethyl)amino]acetate | |
| (180) | tert-Butyl [(1-biphenyl-4-yl-5-pyridin-3-yl-1H-pyrazol-4- | 9.20E−07 |
| | ylmethyl)methylamino]acetate | |
| (181) | (1-Biphenyl-4-yl-5-phenyl-1H-pyrazol-4-ylmethyl)-(5- | 2.40E−07 |
| | methylisoxazol-3-ylmethyl)amine | |
| (182) | Ethyl 4-[1-biphenyl-4-yl-5-(2-fluorophenyl)-1H-pyrazol- | 5.80E−08 |
| | 4-ylmethyl]piperazine-1-carboxylate | |
| (183) | Methyl 3-[(1-biphenyl-4-yl-5-pyridin-3-yl-1H-pyrazol-4- | 8.30E−07 |
| | ylmethyl)amino]propionate | |
| (184) | 1-[1-Biphenyl-4-yl-5-(2-fluorophenyl)-1H-pyrazol-4-ylmethyl]- | 7.50E−08 |
| | 4-pyridin-4-ylmethylpiperazine | |
| (185) | 4-{2-[1-Biphenyl-4-yl-5-(2-fluorophenyl)-1H-pyrazol-4- | 4.00E−08 |
| | yl]ethyl}morpholine | |
| (186) | 5-{[1-Biphenyl-4-yl-5-(2-fluorophenyl)-1H-pyrazol-4-ylmethyl]- | 6.30E−07 |
| | amino}-3H-imidazole-4-carboxamide | |
| (187) | [1-Biphenyl-4-yl-5-(2-fluorophenyl)-1H-pyrazol-4-ylmethyl]- | 5.90E−07 |
| | (1,3,5-trimethyl-1H-pyrazol-4-yl)amine | |
| (188) | 2-[1-Biphenyl-4-yl-5-(2-fluorophenyl)-1H-pyrazol-4-yl]- | 1.40E−06 |
| | ethylamine | |
| (189) | 1-Biphenyl-4-yl-4-chloromethyl-5-(2-fluorophenyl)-1H- | 1.10E−06 |
| | pyrazole | |
| (190) | tert-Butyl 6-{[1-biphenyl-4-yl-5-(2-fluorophenyl)-1H- | 5.20E−07 |
| | pyrazol-4-ylmethyl]amino}-3-azabicyclo[3.1.0]hexane- | |
| | 3-carboxylate | |
| (191) | 1-[1-Biphenyl-4-yl-5-(2-fluorophenyl)-1H-pyrazol-4-ylmethyl]- | 2.00E−07 |
| | 1H-pyridin-2-one | |
| (192) | (3-Azabicyclo[3.1.0]hex-6-yl)-[1-biphenyl-4-yl-5-(2- | 3.20E−07 |
| | fluorophenyl)-1H-pyrazol-4-ylmethyl]amine | |
| (193) | 1-[1-Biphenyl-4-yl-5-(2-fluorophenyl)-1H-pyrazol-4-yl]- | 3.60E−07 |
| | 1-morpholin-4-ylmethanone | |
| (194) | N⁵-{5-(2-Fluorophenyl)-1-[6-(4-fluorophenyl)pyridin-3- | 5.90E−07 |
| | yl]-1H-pyrazol-4-ylmethyl}pyridine-2,5-diamine | |
| (195) | 3-{1-[1-Biphenyl-4-yl-5-(2-fluorophenyl)-1H-pyrazol-4- | 6.60E−08 |
| | ylmethyl]piperidin-4-ylmethyl}pyridine | |
| (196) | {5-(2-Fluorophenyl)-1-[6-(4-fluorophenyl)pyridin-3-yl]- | 1.20E−06 |
| | 1H-pyrazol-4-ylmethyl}pyrazin-2-ylamine | |
| (197) | N-{5-(2-Fluorophenyl)-1-[6-(4-fluorophenyl)pyridin-3-yl]- | 1.30E−06 |
| | 1H-pyrazol-4-ylmethyl}pyrimidine-2,5-diamine | |
| (198) | 1-Methyl-4-[1-(6-phenylpyridin-3-yl)-5-pyridin-3-yl-1H- | 1.40E−07 |
| | pyrazol-4-ylmethyl]piperazine | |
| (199) | Ethyl 1-biphenyl-4-yl-5-(2-fluorophenyl)-1H-pyrazol-4- | |
| | carboxylate | |
| (200) | [1-Biphenyl-4-yl-5-(2-fluorophenyl)-1H-pyrazol-4-yl- | |
| | methylene]-(4-methylpiperazin-1-yl)amine | |
| (201) | 1-[1-Biphenyl-4-yl-5-(2-fluorophenyl)-1H-pyrazol-4-ylmethyl]- | |
| | 4-methylpiperazine | |
| (202) | tert-Butyl {[1-biphenyl-4-yl-5-(2-fluorophenyl)-1H-pyrazol- | |
| | 4-ylmethyl]amino}acetate | |
| (203) | 3-[1-Biphenyl-4-yl-5-(2-fluorophenyl)-1H-pyrazol-4-ylmethyl]- | |
| | thiazolidine | |
| (204) | 4-[1-Biphenyl-4-yl-5-(2-fluorophenyl)-1H-pyrazol-4-ylmethyl]- | |
| | 2,6-dimethylmorpholine | |
| (205) | 3-[1-Biphenyl-4-yl-5-(2-fluorophenyl)-1H-pyrazol-4-yl]- | |
| | acrylic acid | |
| (206) | Ethyl 3-[1-biphenyl-4-yl-5-(2-fluorophenyl)-1H-pyrazol- | |
| | 4-yl]acrylate | |
| (207) | 3-[1-Biphenyl-4-yl-5-(2-fluorophenyl)-1H-pyrazol-4-yl]- | |
| | prop-2-en-1-ol | |
| (208) | Ethyl 1-biphenyl-4-yl-5-(2-fluorophenyl)-1H-pyrazole- | |
| | 3-carboxylate | |
| (209) | [1-Biphenyl-4-yl-5-(2-fluorophenyl)-1H-pyrazol-3-yl]- | |
| | methanol | |
| (210) | tert-Butyl 1-[1-biphenyl-4-yl-5-(2-fluorophenyl)-1H- | |
| | pyrazol-4-ylmethyl]pyrrolidine-2-carboxylate | |
| (211) | tert-Butyl 2-{[1-biphenyl-4-yl-5-(2-fluorophenyl)-1H- | |
| | pyrazol-4-ylmethyl]amino}propionate | |
| (212) | 1-[1-Biphenyl-4-yl-5-(2-fluorophenyl)-1H-pyrazol-4-ylmethyl]- | |
| | 3-(3-methoxyphenyl)piperidine | |
| (213) | 1-[1-Biphenyl-4-yl-5-(2-fluorophenyl)-1H-pyrazol-4-ylmethyl]- | |
| | 3-cyclohexylmethylpiperidine | |
| (214) | 8-[1-Biphenyl-4-yl-5-(2-fluorophenyl)-1H-pyrazol-4-ylmethyl]- | |
| | 1,4-dioxa-8-azaspiro[4.5]decane | |
| (215) | tert-Butyl 2-{[1-biphenyl-4-yl-5-(2-fluorophenyl)-1H- | |
| | pyrazol-4-ylmethyl]amino}-3-methylbutyrate | |
| (216) | 1-[1-Biphenyl-4-yl-5-(2-fluorophenyl)-1H-pyrazol-4-ylmethyl]- | |
| | 2-methylpiperidine | |
| (217) | Ethyl 5-(2-fluorophenyl)-1-(4-nitrophenyl)-1H-pyrazole- | |
| | 4-carboxylate | |
| (218) | Ethyl 1-(4-cyano-phenyl)-5-(2-fluorophenyl)-1H-pyrazole- | |
| | 4-carboxylate | |
| (219) | Ethyl 5-(2-fluorophenyl)-1-[4-(1H-tetrazol-5-yl)phenyl]- | |
| | 1H-pyrazole-4-carboxylate | |
| (220) | 1-[1-Biphenyl-4-yl-5-(2-fluorophenyl)-1H-pyrazol-4-ylmethyl]- | |
| | piperidin-4-one | |
| (221) | tert-Butyl {[1-biphenyl-4-yl-5-(2-fluorophenyl)-1H-pyrazol- | |
| | 4-ylmethyl]methylamino}acetate | |
| (222) | Ethyl 5-(2-fluorophenyl)-1-[4-(N-hydroxycarbamimidoyl)- | |
| | phenyl]-1H-pyrazole-4-carboxylate | |
| (223) | Ethyl 5-(2-fluorophenyl)-1-[4-(5-methyl-[1,2,4]oxadiazol- | |
| | 3-yl)phenyl]-1H-pyrazole-4-carboxylate | |
| (224) | 1-Biphenyl-4-yl-5-(2-fluorophenyl)-1H-pyrazole-4-carbaldehyde | |
| | O-methyl oxime | |
| (225) | 1-Biphenyl-4-yl-5-(2-fluorophenyl)-1H-pyrazole-4-carbaldehyde | |
| | O-allyl oxime | |
| (226) | 4-[5-(2-Fluorophenyl)-1-(3′,4′,5′-trimethoxybiphenyl-4- | |
| | yl)-1H-pyrazol-4-ylmethyl]morpholine | |
| (227) | 4-[5-(2-Fluorophenyl)-1-(4′-trifluoromethylbiphenyl-4- | |
| | yl)-1H-pyrazol-4-ylmethyl]morpholine | |
| (228) | 4′-[5-(2-Fluorophenyl)-4-morpholin-4-ylmethylpyrazol- | |
| | 1-yl]biphenyl-2-carbonitrile | |
| (229) | 4-[1-(2′-Chlorobiphenyl-4-yl)-5-(2-fluorophenyl)-1H- | |
| | pyrazol-4-ylmethyl]morpholine | |
| (230) | 4-[1-(3′,5′-Dichlorobiphenyl-4-yl)-5-(2-fluorophenyl)- | |
| | 1H-pyrazol-4-ylmethyl]morpholine | |
| (231) | 4-[5-(2-Fluorophenyl)-1-(4′-methoxybiphenyl-4-yl)-1H- | |
| | pyrazol-4-ylmethyl]morpholine | |
| (232) | 4-[1-(3′,4′-Difluorobiphenyl-4-yl)-5-(2-fluorophenyl)-1H- | |
| | pyrazol-4-ylmethyl]morpholine | |
| (233) | 4-[5-(2-Fluorophenyl)-1-(4′-methylbiphenyl-4-yl)-1H- | |
| | pyrazol-4-ylmethyl]morpholine | |
| (234) | 4-[5-(2-Fluorophenyl)-1-(3′-methoxybiphenyl-4-yl)-1H- | |
| | pyrazol-4-ylmethyl]morpholine | |
| (235) | 4-[1-(3′-Chlorobiphenyl-4-yl)-5-(2-fluorophenyl)-1H- | |
| | pyrazol-4-ylmethyl]morpholine | |
| (236) | 4-[5-(2-Fluorophenyl)-1-(2′-trifluoromethylbiphenyl-4- | |
| | yl)-1H-pyrazol-4-ylmethyl]morpholine | |
| (237) | 4-[5-(2-Fluorophenyl)-1-(2′-methoxybiphenyl-4-yl)-1H- | |
| | pyrazol-4-ylmethyl]morpholine | |
| (238) | 4-[1-(3′-Ethoxybiphenyl-4-yl)-5-(2-fluorophenyl)-1H- | |
| | pyrazol-4-ylmethyl]morpholine | |
| (239) | 4-[1-(2′-Fluorobiphenyl-4-yl)-5-(2-fluorophenyl)-1H- | |
| | pyrazol-4-ylmethyl]morpholine | |
| (240) | 4-[1-[4-(2,3-Dihydrobenzo[1,4]dioxin-6-yl)phenyl]-5-(2- | |
| | fluorophenyl)-1H-pyrazol-4-ylmethyl]morpholine | |
| (241) | 4-[5-(2-Fluorophenyl)-1-(4-thiophen-3-ylphenyl)-1H- | |
| | pyrazol-4-ylmethyl]morpholine | |
| (242) | 4-[1-(4-Butylphenyl)-5-(2-fluorophenyl)-1H-pyrazol-4- | |
| | ylmethyl]morpholine | |
| (243) | 4′-[5-(2-Fluorophenyl)-4-morpholin-4-ylmethylpyrazol- | |
| | 1-yl]biphenyl-4-carbonitrile | |
| (244) | 4′-[5-(2-Fluorophenyl)-4-morpholin-4-ylmethylpyrazol- | |
| | 1-yl]biphenyl-3-carbonitrile | |
| (245) | 4-[1-(3′,5′-Difluorobiphenyl-4-yl)-5-(2-fluorophenyl)-1H- | |
| | pyrazol-4-ylmethyl]morpholine | |
| (246) | 4-[1-(2′,4′-Difluorobiphenyl-4-yl)-5-(2-fluorophenyl)-1H- | |
| | pyrazol-4-ylmethyl]morpholine | |
| (247) | 4-[1-(2′,5′-Difluorobiphenyl-4-yl)-5-(2-fluorophenyl)-1H- | |
| | pyrazol-4-ylmethyl]morpholine | |
| (248) | 4-[1-(4′-Chlorobiphenyl-4-yl)-5-(2-fluorophenyl)-1H- | |
| | pyrazol-4-ylmethyl]morpholine | |
| (249) | 4-[5-(2-Fluorophenyl)-1-(3′,4′,5′-trifluorobiphenyl-4-yl)- | |
| | 1H-pyrazol-4-ylmethyl]morpholine | |
| (250) | Ethyl 5-(2-fluorophenyl)-1-(4-trifluoromethylphenyl)- | |
| | 1H-pyrazole-4-carboxylate | |
| (251) | 4-[5-(2-Fluorophenyl)-1-p-tolyl-1H-pyrazol-4-ylmethyl]- | |
| | morpholine | |
| (252) | {[1-Biphenyl-4-yl-5-(2-fluorophenyl)-1H-pyrazol-4-ylmethyl]- | |
| | methylamino}acetic acid | |
| (253) | 1-[1-Biphenyl-4-yl-5-(2-fluorophenyl)-1H-pyrazol-4-ylmethyl]- | |
| | pyrrolidine-2-carboxylic acid | |
| (254) | 2-{[1-Biphenyl-4-yl-5-(2-fluorophenyl)-1H-pyrazol-4-ylmethyl]- | |
| | amino}-3-methylbutyric acid | |
| (255) | 2-{[1-Biphenyl-4-yl-5-(2-fluorophenyl)-1H-pyrazol-4-ylmethyl]- | |
| | amino}propionic acid | |
| (256) | {[1-Biphenyl-4-yl-5-(2-fluorophenyl)-1H-pyrazol-4-ylmethyl]- | |
| | methylamino}morpholin-4-ylethanone | |
| (257) | Ethyl 5-(2-fluorophenyl)-1-(4-imidazol-1-ylphenyl)-1H- | |
| | pyrazole-4-carboxylate | |
| (258) | [5-(2-Fluorophenyl)-1-(4-imidazol-1-ylphenyl)-1H- | |
| | pyrazol-4-yl]methanol | |
| (259) | 1-[1-Biphenyl-4-yl-5-(2-fluorophenyl)-1H-pyrazol-4-ylmethyl]- | |
| | pyrrolidine-2-carboxamide | |
| (260) | [5-(2-Fluorophenyl)-1-(4-trifluoromethylphenyl)-1H- | |
| | pyrazol-4-yl]methanol | |
| (261) | Ethyl {[1-biphenyl-4-yl-5-(2-fluorophenyl)-1H-pyrazol- | |
| | 4-ylmethyl]amino}acetate | |
| (262) | tert-Butyl (2-{[1-biphenyl-4-yl-5-(2-fluorophenyl)-1H- | |
| | pyrazol-4-ylmethyl]amino}ethyl)carbamate | |
| (263) | tert-Butyl 4-{[1-biphenyl-4-yl-5-(2-fluorophenyl)-1H- | |
| | pyrazol-4-ylmethyl]amino}piperidine-1-carboxylate | |
| (264) | Ethyl 1-[1-biphenyl-4-yl-5-(2-fluorophenyl)-1H-pyrazol- | |
| | 4-ylmethyl]piperidine-4-carboxylate | |
| (265) | 4-[5-(2-Fluorophenyl)-1-(4-imidazol-1-ylphenyl)-1H- | |
| | pyrazol-4-ylmethyl]morpholine | |
| (266) | 1-[5-(2-Fluorophenyl)-1-(4-imidazol-1-ylphenyl)-1H- | |
| | pyrazol-4-ylmethyl]-4-methylpiperazine | |
| (267) | Ethyl {[5-(2-fluorophenyl)-1-(4-imidazol-1-ylphenyl)- | |
| | 1H-pyrazol-4-ylmethyl]amino}acetate | |
| (268) | 1-[1-Biphenyl-4-yl-5-(2-fluorophenyl)-1H-pyrazol-4-ylmethyl]- | |
| | piperidine-4-carboxylic acid | |
| (269) | {[1-Biphenyl-4-yl-5-(2-fluorophenyl)-1H-pyrazol-4-ylmethyl]- | |
| | amino}acetic acid | |
| (270) | tert-Butyl 5-[1-biphenyl-4-yl-5-(2-fluorophenyl)-1H- | |
| | pyrazol-4-ylmethyl]-2,5-diazabicyclo[2.2.1]heptane-2- | |
| | carboxylate | |
| (271) | 4-[1-Biphenyl-4-yl-5-(2-fluorophenyl)-1H-pyrazol-4-ylmethyl]- | |
| | piperazine-1-carbaldehyde | |
| (272) | Ethyl {1-[1-biphenyl-4-yl-5-(2-fluorophenyl)-1H-pyrazol- | |
| | 4-ylmethyl]-3-oxopiperazin-2-yl}acetate | |
| (273) | 4-[1-Biphenyl-4-yl-5-(2-fluorophenyl)-1H-pyrazol-4-ylmethyl]- | |
| | thiomorpholine | |
| (274) | [1-Biphenyl-4-yl-5-(2-fluorophenyl)-1H-pyrazol-4-ylmethyl]- | |
| | pyridin-3-ylamine | |
| (275) | 1-[1-Biphenyl-4-yl-5-(2-fluorophenyl)-1H-pyrazol-4-ylmethyl]- | |
| | imidazolidin-2-one | |
| (276) | 4-[1-Biphenyl-4-yl-5-(2-fluorophenyl)-1H-pyrazol-4-ylmethyl]- | |
| | thiomorpholine 1-oxide | |
| (277) | Dimethyl 2-{[1-biphenyl-4-yl-5-(2-fluorophenyl)-1H- | |
| | pyrazol-4-ylmethyl]amino}succinate | |
| (278) | 4-[1-(2′,6′-Difluorobiphenyl-4-yl)-5-(2-fluorophenyl)-1H- | |
| | pyrazol-4-ylmethyl]morpholine | |
| (279) | 2-{[1-Biphenyl-4-yl-5-(2-fluorophenyl)-1H-pyrazol-4-ylmethyl]- | |
| | amino}malonamide | |
| (280) | Ethyl [1-biphenyl-4-yl-5-(2-fluorophenyl)-1H-pyrazol-4- | |
| | ylmethyl]carbamoylmethylcarbamate | |
| (281) | 4-[1-Biphenyl-4-yl-5-(2-fluorophenyl)-1H-pyrazol-4-ylmethyl]- | |
| | morpholine-3,5-dione | |
| (282) | 1-[1-Biphenyl-4-yl-5-(2-fluorophenyl)-1H-pyrazol-4-ylmethyl]- | |
| | piperidin-4-one O-methyl oxime | |
| (283) | 4-[5-(2-Fluorophenyl)-1-(4-isopropylphenyl)-1H-pyrazol- | |
| | 4-ylmethyl]morpholine | |
| (284) | Ethyl {[5-(2-fluorophenyl)-1-(4-isopropylphenyl)-1H- | |
| | pyrazol-4-ylmethyl]amino}acetate | |
| (285) | 4-[5-(2-Fluorophenyl)-1-(4-trifluoromethoxyphenyl)-1H- | |
| | pyrazol-4-ylmethyl]morpholine | |
| (286) | Ethyl {[5-(2-fluorophenyl)-1-(4-trifluoromethoxyphenyl)- | |
| | 1H-pyrazol-4-ylmethyl]amino}acetate | |
| (287) | Ethyl 5-(2-fluorophenyl)-1-(6-phenylpyridin-3-yl)-1H- | |
| | pyrazole-4-carboxylate | |
| (288) | [5-(2-Fluorophenyl)-1-(6-phenylpyridin-3-yl)-1H-pyrazol- | |
| | 4-yl]methanol | |
| (289) | 4-[5-(2-Fluorophenyl)-1-(6-phenylpyridin-3-yl)-1H- | |
| | pyrazol-4-ylmethyl]morpholine | |
| (290) | tert-Butyl {[5-(2-fluorophenyl)-1-(6-phenylpyridin-3-yl)- | |
| | 1H-pyrazol-4-ylmethyl]amino}acetate | |
| (291) | {[5-(2-Fluorophenyl)-1-(6-phenylpyridin-3-yl)-1H-pyrazol- | |
| | 4-ylmethyl]amino}acetic acid | |
| (292) | 1-[5-(2-Fluorophenyl)-1-(6-phenylpyridin-3-yl)-1H- | |
| | pyrazol-4-ylmethyl]piperazine | |
| (293) | 1-[5-(2-Fluorophenyl)-1-(6-phenylpyridin-3-yl)-1H- | |
| | pyrazol-4-ylmethyl]-4-methylpiperazine | |
| (294) | tert-Butyl 4-[5-(2-fluorophenyl)-1-(6-phenylpyridin-3- | |
| | yl)-1H-pyrazol-4-ylmethyl]piperazine-1-carboxylate | |
| (295) | Ethyl 1-[5-(2-fluorophenyl)-1-(6-phenylpyridin-3-yl)-1H- | |
| | pyrazol-4-ylmethyl]piperidine-4-carboxylate | |
| (296) | 2-{4-[5-(2-Fluorophenyl)-1-(6-phenylpyridin-3-yl)-1H- | |
| | pyrazol-4-ylmethyl]piperazin-1-yl}nicotinonitrile | |
| (297) | tert-Butyl (2-{[5-(2-fluorophenyl)-1-(6-phenylpyridin-3- | |
| | yl)-1H-pyrazol-4-ylmethyl]amino}ethyl)carbamate | |
| (298) | tert-Butyl 4-{[5-(2-fluorophenyl)-1-(6-phenylpyridin-3- | |
| | yl)-1H-pyrazol-4-ylmethyl]amino}piperidine-1-carboxylate | |
| (299) | Methyl 5-{[5-(2-fluorophenyl)-1-(6-phenylpyridin-3-yl)- | |
| | 1H-pyrazol-4-ylmethyl]amino}furan-2-carboxylate | |
| (300) | Ethyl 4-{[5-(2-fluorophenyl)-1-(6-phenylpyridin-3-yl)- | |
| | 1H-pyrazol-4-ylmethyl]amino}piperidine-1-carboxylate | |
| (301) | N1-[5-(2-fluorophenyl)-1-(6-phenylpyridin-3-yl)-1H- | |
| | pyrazol-4-ylmethyl]ethane-1,2-diamine | |
| (302) | [5-(2-Fluorophenyl)-1-(6-phenylpyridin-3-yl)-1H-pyrazol- | |
| | 4-ylmethyl]piperidin-4-ylamine | |
| (303) | 1-[5-(2-Fluorophenyl)-1-(6-phenylpyridin-3-yl)-1H- | |
| | pyrazol-4-ylmethyl]piperidine-4-carboxylic acid | |
| (304) | 4-Ethyl-1-[5-(2-fluorophenyl)-1-(6-phenylpyridin-3-yl)- | |
| | 1H-pyrazol-4-ylmethyl]piperidin-4-ol | |
| (305) | 5-[5-(2-Fluorophenyl)-1-(6-phenylpyridin-3-yl)-1H- | |
| | pyrazol-4-ylmethyl]-2-oxa-5-azabicyclo[2.2.1]heptane | |
| (306) | Ethyl {4-[5-(2-fluorophenyl)-1-(6-phenylpyridin-3-yl)- | |
| | 1H-pyrazol-4-ylmethyl]piperazin-1-yl}acetate | |
| (307) | {4-[5-(2-Fluorophenyl)-1-(6-phenylpyridin-3-yl)-1H- | |
| | pyrazol-4-ylmethyl]piperazin-1-yl}acetic acid | |
| (308) | 5-[5-(2-Fluorophenyl)-4-piperidin-1-ylmethylpyrazol-1- | |
| | yl]-2-phenylpyridine | |
| (309) | 4-{5-(2-Fluorophenyl)-1-[6-(4-fluorophenyl)pyridin-3- | |
| | yl]-1H-pyrazol-4-ylmethyl}morpholine | |
| (310) | Ethyl ({5-(2-fluorophenyl)-1-[6-(4-fluorophenyl)pyridin- | |
| | 3-yl]-1H-pyrazol-4-ylmethyl}amino)acetate | |
| (311) | {[1-Biphenyl-4-yl-5-(2-fluorophenyl)-1H-pyrazol-4-ylmethyl]- | |
| | amino}acetic acid | |
| (312) | tert-Butyl {[1-biphenyl-4-yl-5-(2-fluorophenyl)-1H-pyrazol- | |
| | 4-ylmethyl]amino}acetate | |
| (313) | tert-Butyl {[5-(2-fluorophenyl)-1-(6-phenylpyridin-3-yl)- | |
| | 1H-pyrazol-4-ylmethyl]amino}acetate | |
| (314) | {[5-(2-Fluorophenyl)-1-(6-phenylpyridin-3-yl)-1H-pyrazol- | |
| | 4-ylmethyl]amino}acetic acid | |
| (315) | tert-Butyl [(1-biphenyl-4-yl-5-phenyl-1H-pyrazol-4-ylmethyl)- | |
| | amino]acetate | |
| (316) | tert-Butyl {[biphenyl-4-yl-(bistrifluoromethylphenyl)-1H- | |
| | pyrazol-4-ylmethyl]amino}acetate | |
| (317) | tert-Butyl 1-[1-biphenyl-4-yl-5-(2-fluorophenyl)-1H- | |
| | pyrazol-4-ylmethyl]pyrrolidine-2-carboxylate | |
| (318) | tert-Butyl 2-{[1-biphenyl-4-yl-5-(2-fluorophenyl)-1H- | |
| | pyrazol-4-ylmethyl]amino}propionate | |
| (319) | tert-Butyl 2-{[1-biphenyl-4-yl-5-(2-fluorophenyl)-1H- | |
| | pyrazol-4-ylmethyl]amino}-3-methylbutyrate | |
| (320) | {[Biphenyl-4-yl-(bistrifluoromethylphenyl)-1H-pyrazol-4- | |
| | ylmethyl]amino}acetic acid | |
| (321) | [(1-Biphenyl-4-yl-5-phenyl-1H-pyrazol-4-ylmethyl)- | |
| | amino]acetic acid | |
| (322) | tert-Butyl {[1-biphenyl-4-yl-5-(2-fluorophenyl)-1H-pyrazol- | |
| | 4-ylmethyl]methylamino}acetate | |
| (323) | {[1-Biphenyl-4-yl-5-(2-fluorophenyl)-1H-pyrazol-4-ylmethyl]- | |
| | methylamino}acetic acid | |
| (324) | 1-[1-Biphenyl-4-yl-5-(2-fluorophenyl)-1H-pyrazol-4-ylmethyl]- | |
| | pyrrolidine-2-carboxylic acid | |
| (325) | 2-{[1-Biphenyl-4-yl-5-(2-fluorophenyl)-1H-pyrazol-4-ylmethyl]- | |
| | amino}-3-methylbutanoic acid | |
| (326) | 2-{[1-Biphenyl-4-yl-5-(2-fluorophenyl)-1H-pyrazol-4-ylmethyl]- | |
| | amino}propionic acid | |
| (327) | {[1-Biphenyl-4-yl-5-(2-fluorophenyl)-1H-pyrazol-4-ylmethyl]- | |
| | methylamino}morpholin-4-ylethanone | |
| (328) | 1-[1-Biphenyl-4-yl-5-(2-fluorophenyl)-1H-pyrazol-4-ylmethyl]- | |
| | pyrrolidine-2-carboxamide | |
| (329) | Ethyl {[1-biphenyl-4-yl-5-(2-fluorophenyl)-1H-pyrazol-4- | |
| | ylmethyl]amino}acetate | |
| (330) | Ethyl {[5-(2-fluorophenyl)-1-(4-imidazol-1-ylphenyl)-1H- | |
| | pyrazol-4-ylmethyl]amino}acetate | |
| (331) | {[1-Biphenyl-4-yl-5-(2-fluorophenyl)-1H-pyrazol-4-ylmethyl]- | |
| | amino}acetic acid | |
| (332) | Ethyl {1-[1-biphenyl-4-yl-5-(2-fluorophenyl)-1H-pyrazol- | |
| | 4-ylmethyl]-3-oxopiperazin-2-yl}acetate | |
| (333) | Dimethyl 2-{[1-biphenyl-4-yl-5-(2-fluorophenyl)-1H- | |
| | pyrazol-4-ylmethyl]amino}succinate | |
| (334) | 2-{[1-Biphenyl-4-yl-5-(2-fluorophenyl)-1H-pyrazol-4-ylmethyl]- | |
| | amino}malonamide | |
| (335) | Ethyl [1-biphenyl-4-yl-5-(2-fluorophenyl)-1H-pyrazol-4- | |
| | ylmethyl]carbamoylmethylcarbamate | |
| (336) | Ethyl {[5-(2-fluorophenyl)-1-(4-isopropylphenyl)-1H- | |
| | pyrazol-4-ylmethyl]amino}acetate | |
| (337) | Ethyl {[5-(2-fluorophenyl)-1-(4-trifluoromethoxyphenyl)- | |
| | 1H-pyrazol-4-ylmethyl]amino}acetate | |
| (338) | Ethyl ({5-(2-fluorophenyl)-1-[6-(4-fluorophenyl)pyridin- | |
| | 3-yl]-1H-pyrazol-4-ylmethyl}amino)acetate | |
| (339) | 4-(1-Biphenyl-4-yl-5-pyridin-2-yl-1H-pyrazol-4-ylmethyl)- | |
| | morpholine | |

### EXAMPLES 340-389

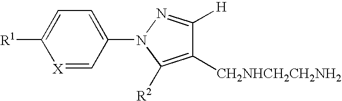

| | R¹ | R² | X |
|---|---|---|---|
| (340) | 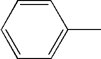 | 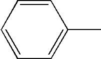 | CH |
| (341) | 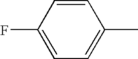 | 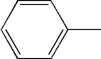 | CH |
| (342) | 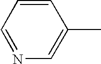 | 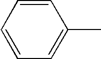 | CH |
| (343) | 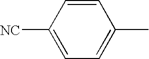 | 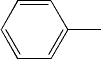 | CH |
| (344) | 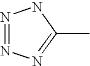 | 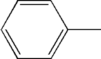 | CH |
| (345) | 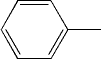 | 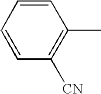 | CH |
| (346) | 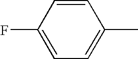 | 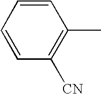 | CH |
| (347) | 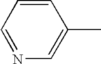 | 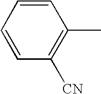 | CH |
| (348) | 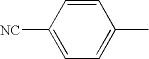 | 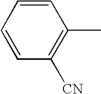 | CH |
| (349) | 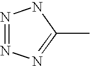 | 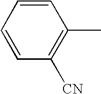 | CH |
| (350) | 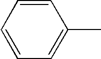 | 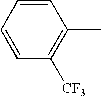 | CH |
| (351) | 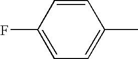 | 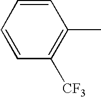 | CH |
| (352) | 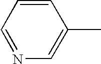 | 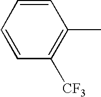 | CH |
| (353) | 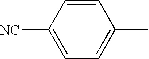 | 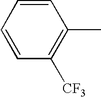 | CH |
| (354) | 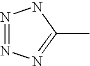 | 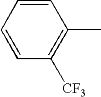 | CH |
| (355) | 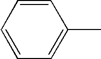 | 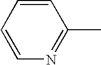 | CH |
| (356) | 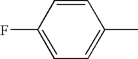 | 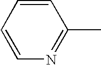 | CH |
| (357) | 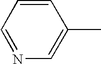 | 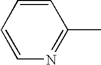 | CH |
| (358) | 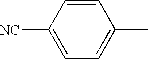 | 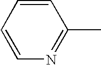 | CH |
| (359) | 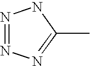 | 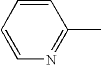 | CH |
| (360) | 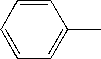 | 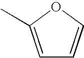 | CH |
| (361) | 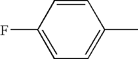 | 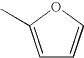 | CH |
| (362) | 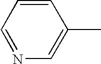 | CF₃ | CH |
| (363) | 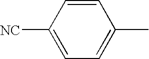 | CF₃ | CH |
| (364) | 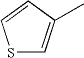 | CF₃ | CH |
| (365) | 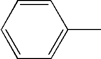 | 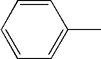 | N |
| (366) | 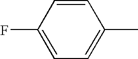 | 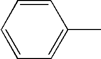 | N |
| (367) | 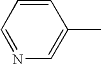 | 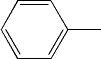 | N |
| (368) | 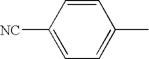 | 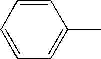 | N |
| (369) | 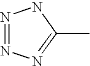 | 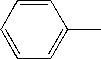 | N |
| (370) | 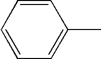 | 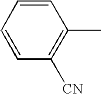 | N |
| (371) | 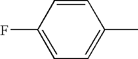 | 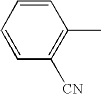 | N |
| (372) | 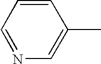 | 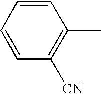 | N |
| (373) | 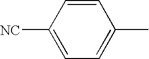 | 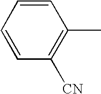 | N |
| (374) | 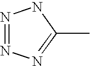 | 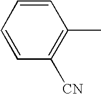 | N |
| (375) | 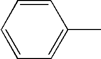 | 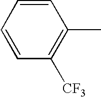 | N |
| (376) | 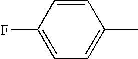 | 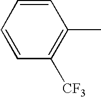 | N |
| (377) | 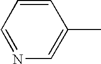 | 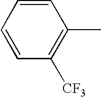 | N |
| (378) | 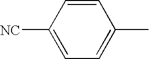 | 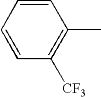 | N |
| (379) | 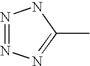 | 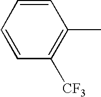 | N |
| (380) | 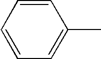 | 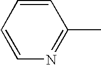 | N |
| (381) | 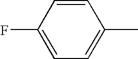 | 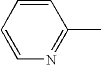 | N |
| (382) | 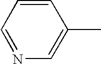 | 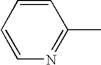 | N |
| (383) | 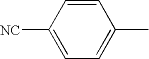 | 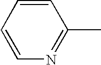 | N |
| (384) | 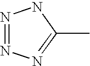 | 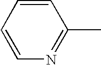 | N |
| (385) | 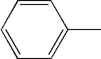 | 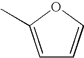 | N |
| (386) | 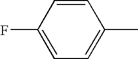 | 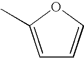 | N |
| (387) | 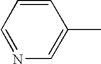 | CF₃ | N |
| (388) | 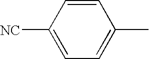 | CF₃ | N |
| (389) | 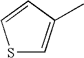 | CF₃ | N |

### EXAMPLES 390-439

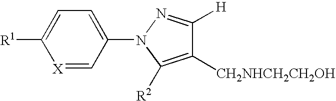

| | R¹ | R² | X |
|---|---|---|---|
| (390) | 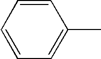 | 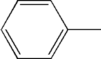 | CH |
| (391) | 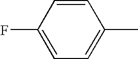 | 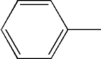 | CH |
| (392) | 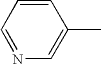 | 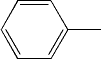 | CH |
| (393) | 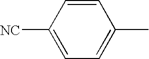 | 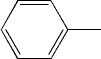 | CH |
| (394) | 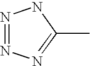 | 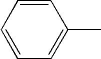 | CH |
| (395) | 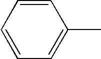 | 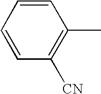 | CH |
| (396) | 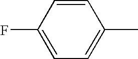 | 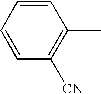 | CH |
| (397) | 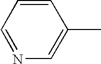 | 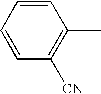 | CH |
| (398) | 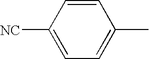 | 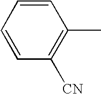 | CH |
| (399) | 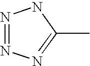 | 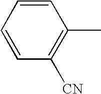 | CH |
| (400) | 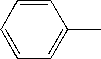 | 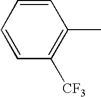 | CH |
| (401) | 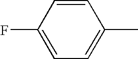 | 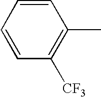 | CH |
| (402) | 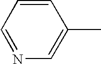 | 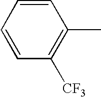 | CH |
| (403) | 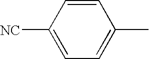 | 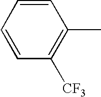 | CH |
| (404) | 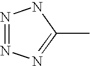 | 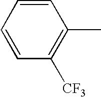 | CH |
| (405) | 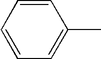 | 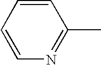 | CH |
| (406) | 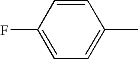 | 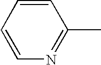 | CH |
| (407) | 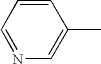 | 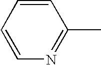 | CH |
| (408) | 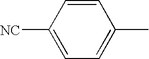 | 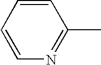 | CH |
| (409) | 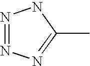 | 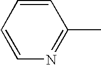 | CH |
| (410) | 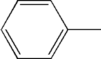 | 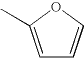 | CH |
| (411) | 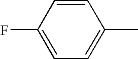 | 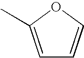 | CH |
| (412) | 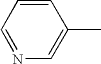 | CF₃ | CH |
| (413) | 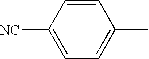 | CF₃ | CH |
| (414) | 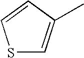 | CF₃ | CH |
| (415) | 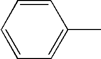 | 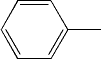 | N |
| (416) | 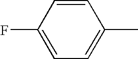 | 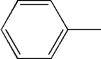 | N |
| (417) | 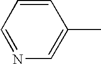 | 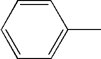 | N |
| (418) | 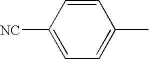 | 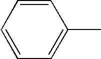 | N |
| (419) | 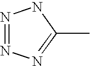 | 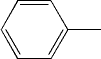 | N |
| (420) | 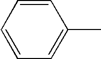 | 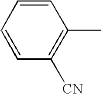 | N |
| (421) | 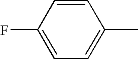 | 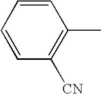 | N |
| (422) | 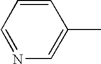 | 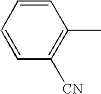 | N |
| (423) | 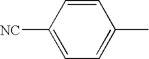 | 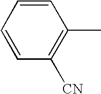 | N |
| (424) | 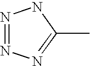 | 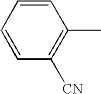 | N |
| (425) | 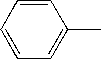 | 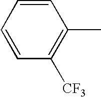 | N |
| (426) | 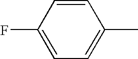 | 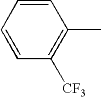 | N |
| (427) | 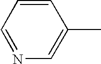 | 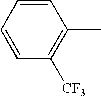 | N |
| (428) | 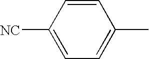 | 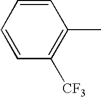 | N |
| (429) | 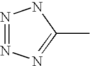 | 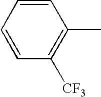 | N |
| (430) | 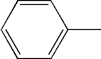 | 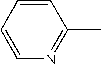 | N |
| (431) | 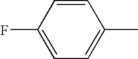 | 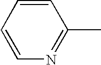 | N |
| (432) | 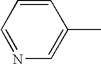 | 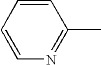 | N |
| (433) | 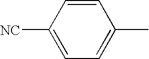 | 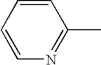 | N |
| (434) | 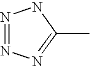 | 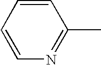 | N |
| (435) | 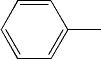 | 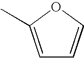 | N |
| (436) | 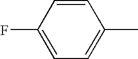 | 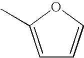 | N |
| (437) | 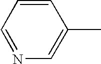 | CF₃ | N |
| (438) | 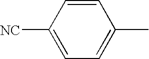 | CF₃ | N |
| (439) | 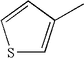 | CF₃ | N |

### EXAMPLES 440-489

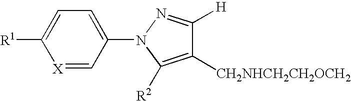

| | R¹ | R² | X |
|---|---|---|---|
| (440) | 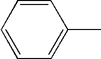 | 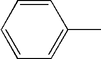 | CH |
| (441) | 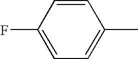 | 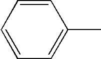 | CH |
| (442) | 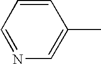 | 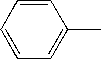 | CH |
| (443) | 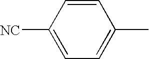 | 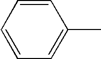 | CH |
| (444) | 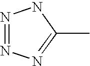 | 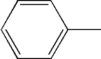 | CH |
| (445) | 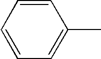 | 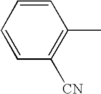 | CH |
| (446) | 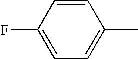 | 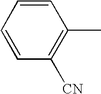 | CH |
| (447) | 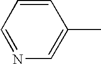 | 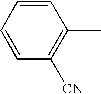 | CH |
| (448) | 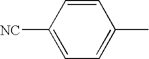 | 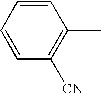 | CH |
| (449) | 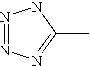 | 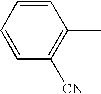 | CH |
| (450) | 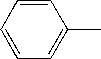 | 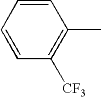 | CH |
| (451) | 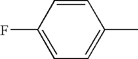 | 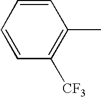 | CH |
| (452) | 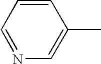 | 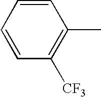 | CH |
| (453) | 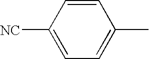 | 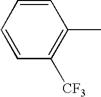 | CH |
| (454) | 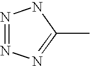 | 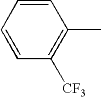 | CH |
| (455) | 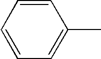 | 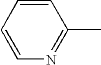 | CH |
| (456) | 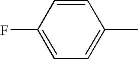 | 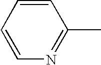 | CH |
| (457) | 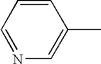 | 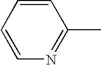 | CH |
| (458) | 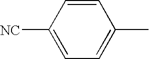 | 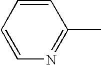 | CH |
| (459) | 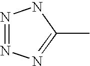 | 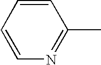 | CH |
| (460) | 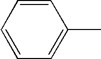 | 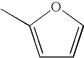 | CH |
| (461) | 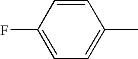 | 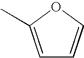 | CH |
| (462) | 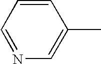 | CF₃ | CH |
| (463) | 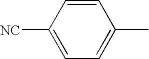 | CF₃ | CH |
| (464) | 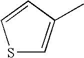 | CF₃ | CH |
| (465) | 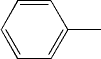 | 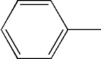 | N |
| (466) | 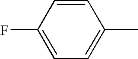 | 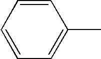 | N |
| (467) | 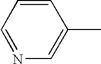 | 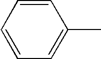 | N |
| (468) | 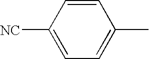 | 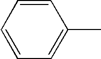 | N |
| (469) | 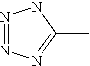 | 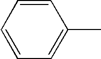 | N |
| (470) | 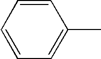 | 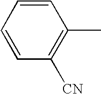 | N |
| (471) | 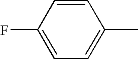 | 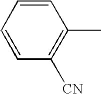 | N |
| (472) | 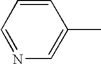 | 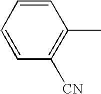 | N |
| (473) | 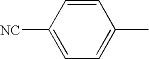 | 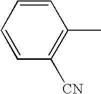 | N |
| (474) | 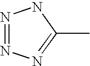 | 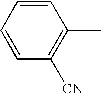 | N |
| (475) | 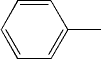 | 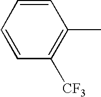 | N |
| (476) | 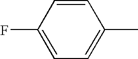 | 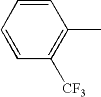 | N |
| (477) | 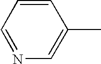 | 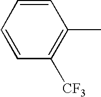 | N |
| (478) | 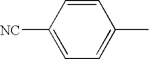 | 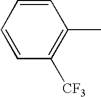 | N |
| (479) | 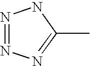 | 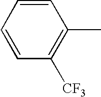 | N |
| (480) | 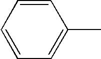 | 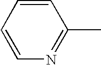 | N |
| (481) | 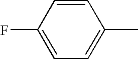 | 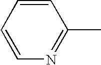 | N |
| (482) | 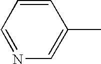 | 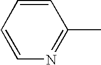 | N |
| (483) | 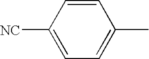 | 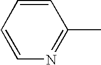 | N |
| (484) | 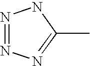 | 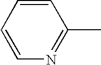 | N |
| (485) | 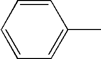 | 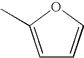 | N |
| (486) | 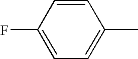 | 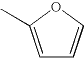 | N |
| (487) | 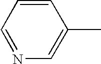 | CF₃ | N |
| (488) | 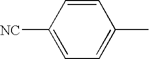 | CF₃ | N |
| (489) | 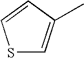 | CF₃ | N |

### EXAMPLES 490-539

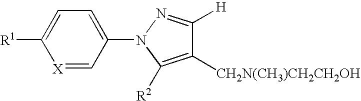

| | R¹ | R² | X |
|---|---|---|---|
| (490) | 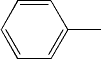 | 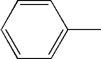 | CH |
| (491) | 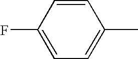 | 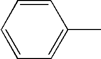 | CH |
| (492) | 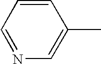 | 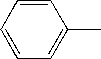 | CH |
| (493) | 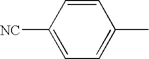 | 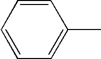 | CH |
| (494) | 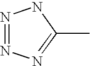 | 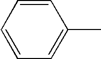 | CH |
| (495) | 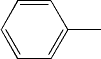 | 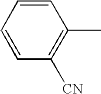 | CH |
| (496) | 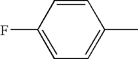 | 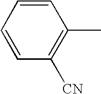 | CH |
| (497) | 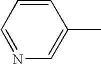 | 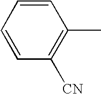 | CH |
| (498) | 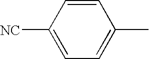 | 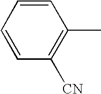 | CH |
| (499) | 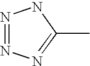 | 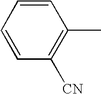 | CH |
| (500) | 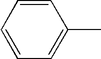 | 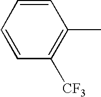 | CH |
| (501) | 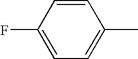 | 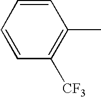 | CH |
| (502) | 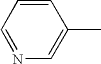 | 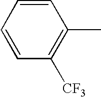 | CH |
| (503) | 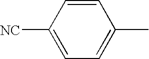 | 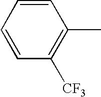 | CH |
| (504) | 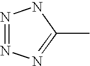 | 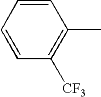 | CH |
| (505) | 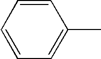 | 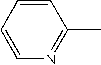 | CH |
| (506) | 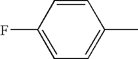 | 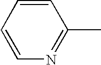 | CH |
| (507) | 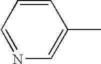 | 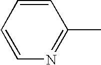 | CH |
| (508) | 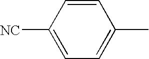 | 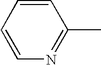 | CH |
| (509) | 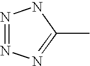 | 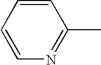 | CH |
| (510) | 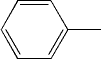 | 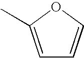 | CH |
| (511) | 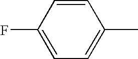 | 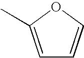 | CH |
| (512) | 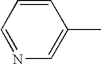 | CF₃ | CH |
| (513) | 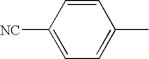 | CF₃ | CH |
| (514) | 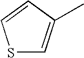 | CF₃ | CH |
| (515) | 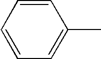 | 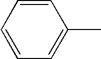 | N |
| (516) | 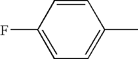 | 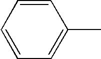 | N |
| (517) | 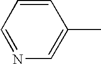 | 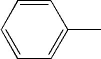 | N |
| (518) | 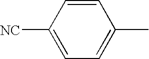 | 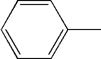 | N |
| (519) | 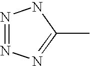 | 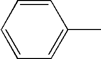 | N |
| (520) | 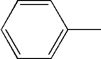 | 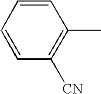 | N |
| (521) | 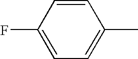 | 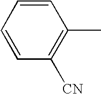 | N |
| (522) | 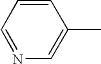 | 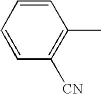 | N |
| (523) | 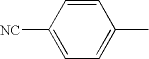 | 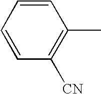 | N |
| (524) | 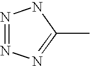 | 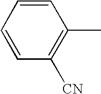 | N |
| (525) | 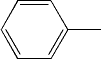 | 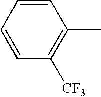 | N |
| (526) | 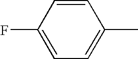 | 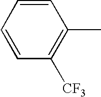 | N |
| (527) | 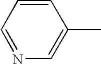 | 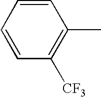 | N |
| (528) | 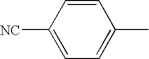 | 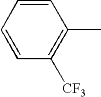 | N |
| (529) | 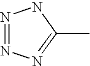 | 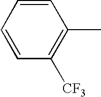 | N |
| (530) | 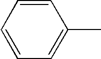 | 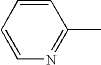 | N |
| (531) | 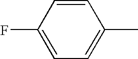 | 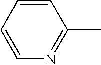 | N |
| (532) | 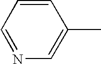 | 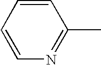 | N |
| (533) | 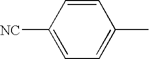 | 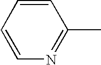 | N |
| (534) | 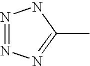 | 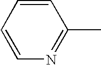 | N |
| (535) | 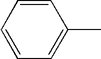 | 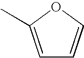 | N |
| (536) | 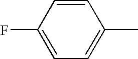 | 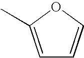 | N |
| (537) | 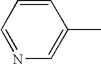 | CF₃ | N |
| (538) | 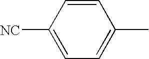 | CF₃ | N |
| (539) | 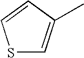 | CF₃ | N |

### EXAMPLES 540-589

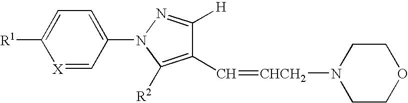

| | R¹ | R² | X |
|---|---|---|---|
| (540) | 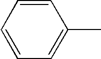 | 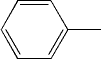 | CH |
| (541) | 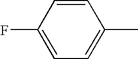 | 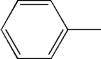 | CH |
| (542) | 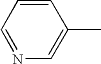 | 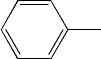 | CH |
| (543) | 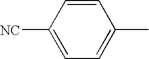 | 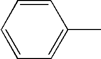 | CH |
| (544) | 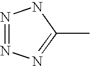 | 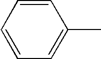 | CH |
| (545) | 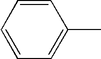 | 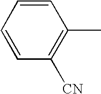 | CH |
| (546) | 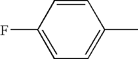 | 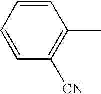 | CH |
| (547) | 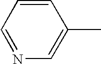 | 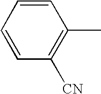 | CH |
| (548) | 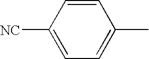 | 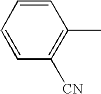 | CH |
| (549) | 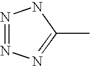 | 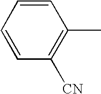 | CH |
| (550) | 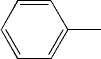 | 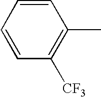 | CH |
| (551) | 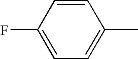 | 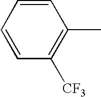 | CH |
| (552) | 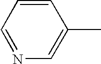 | 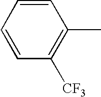 | CH |
| (553) | 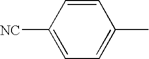 | 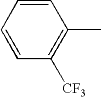 | CH |
| (554) | 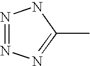 | 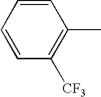 | CH |
| (555) | 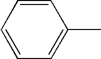 | 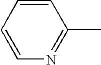 | CH |
| (556) | 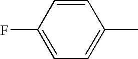 | 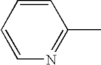 | CH |
| (557) | 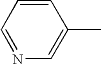 | 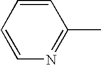 | CH |
| (558) | 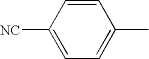 | 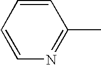 | CH |
| (559) | 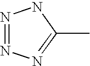 | 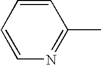 | CH |
| (560) | 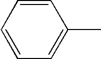 | 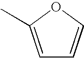 | CH |
| (561) | 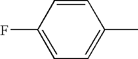 | 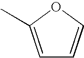 | CH |
| (562) | 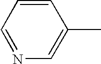 | CF₃ | CH |
| (563) | 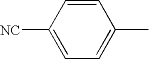 | CF₃ | CH |
| (564) | 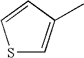 | CF₃ | CH |
| (565) | 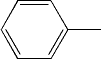 | 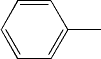 | N |
| (566) | 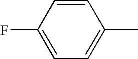 | 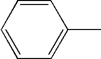 | N |
| (567) | 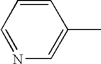 | 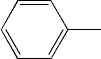 | N |
| (568) | 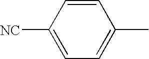 | 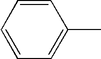 | N |
| (569) | 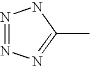 | 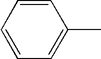 | N |
| (570) | 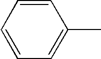 | 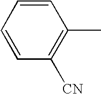 | N |
| (571) | 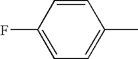 | 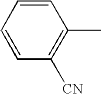 | N |
| (572) | 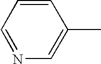 | 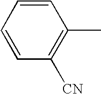 | N |
| (573) | 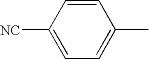 | 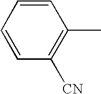 | N |
| (574) | 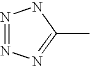 | 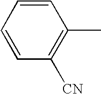 | N |
| (575) | 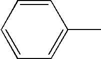 | 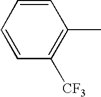 | N |
| (576) | 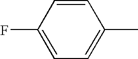 | 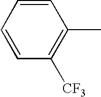 | N |
| (577) | 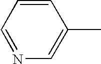 | 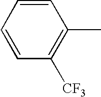 | N |
| (578) | 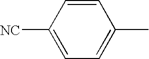 | 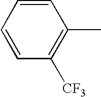 | N |
| (579) | 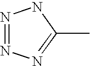 | 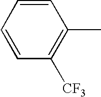 | N |
| (580) | 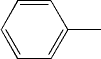 | 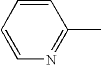 | N |
| (581) | 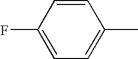 | 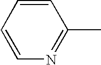 | N |
| (582) | 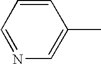 | 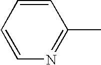 | N |
| (583) | 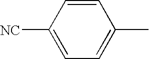 | 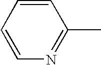 | N |
| (584) | 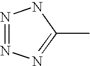 | 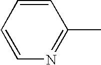 | N |
| (585) | 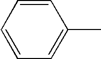 | 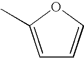 | N |
| (586) | 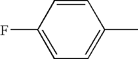 | 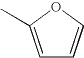 | N |
| (587) | 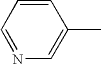 | CF₃ | N |
| (588) | 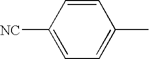 | CF₃ | N |
| (589) | 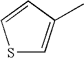 | CF₃ | N |

### EXAMPLES 590-639

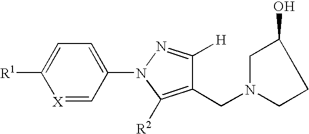

| | R¹ | R² | X |
|---|---|---|---|
| (590) | 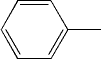 | 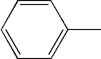 | CH |
| (591) | 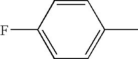 | 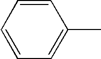 | CH |
| (592) | 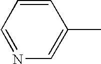 | 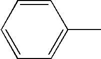 | CH |
| (593) | 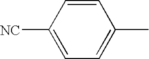 | 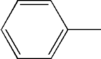 | CH |
| (594) | 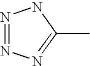 | 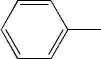 | CH |
| (595) | 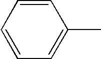 | 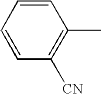 | CH |
| (596) | 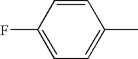 | 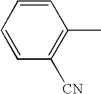 | CH |
| (597) | 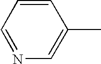 | 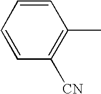 | CH |
| (598) | 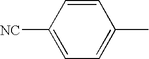 | 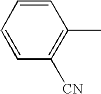 | CH |
| (599) | 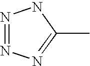 | 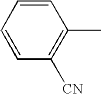 | CH |
| (600) | 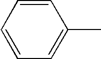 | 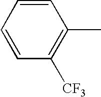 | CH |
| (601) | 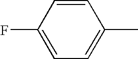 | 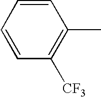 | CH |
| (602) | 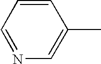 | 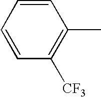 | CH |
| (603) | 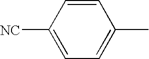 | 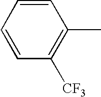 | CH |
| (604) | 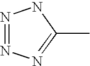 | 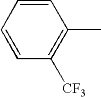 | CH |
| (605) | 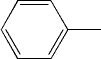 | 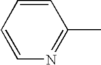 | CH |
| (606) | 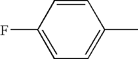 | 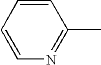 | CH |
| (607) | 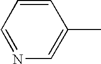 | 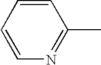 | CH |
| (608) | 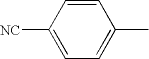 | 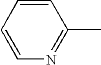 | CH |
| (609) | 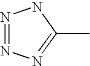 | 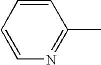 | CH |
| (610) | 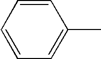 | 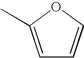 | CH |
| (611) | 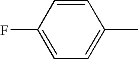 | 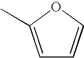 | CH |
| (612) | 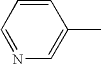 | CF₃ | CH |
| (613) | 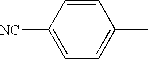 | CF₃ | CH |
| (614) | 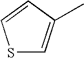 | CF₃ | CH |
| (615) | 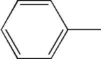 | 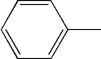 | N |
| (616) | 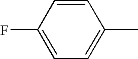 | 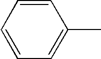 | N |
| (617) | 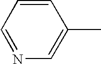 | 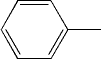 | N |
| (618) | 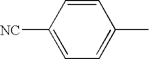 | 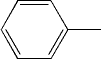 | N |
| (619) | 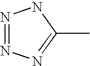 | 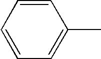 | N |
| (620) | 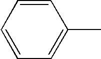 | 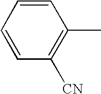 | N |
| (621) | 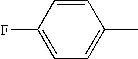 | 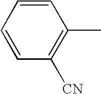 | N |
| (622) | 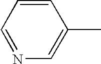 | 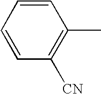 | N |
| (623) | 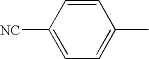 | 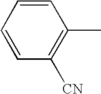 | N |
| (624) | 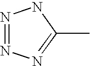 | 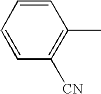 | N |
| (625) | 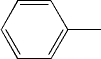 | 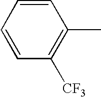 | N |
| (626) | 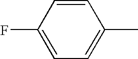 | 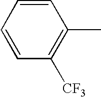 | N |
| (627) | 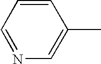 | 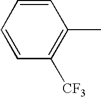 | N |
| (628) | 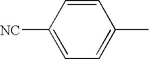 | 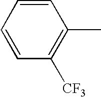 | N |
| (629) | 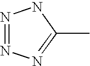 | 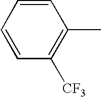 | N |
| (630) | 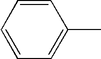 | 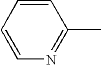 | N |
| (631) | 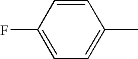 | 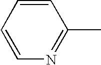 | N |
| (632) | 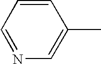 | 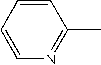 | N |
| (633) | 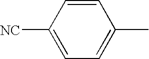 | 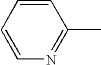 | N |
| (634) | 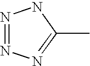 | 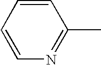 | N |
| (635) | 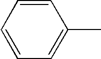 | 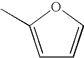 | N |
| (636) | 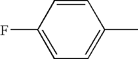 | 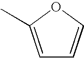 | N |
| (637) | 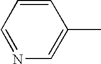 | CF₃ | N |
| (638) | 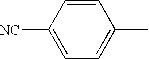 | CF₃ | N |
| (639) | 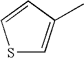 | CF₃ | N |

### EXAMPLES 640-689

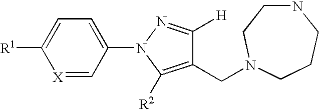

| | R¹ | R² | X |
|---|---|---|---|
| (640) | 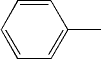 | 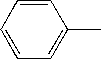 | CH |
| (641) | 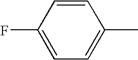 | 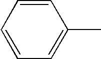 | CH |
| (642) | 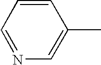 | 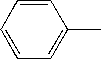 | CH |
| (643) | 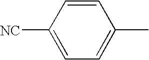 | 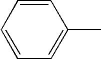 | CH |
| (644) | 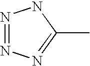 | 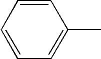 | CH |
| (645) | 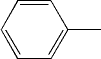 | 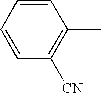 | CH |
| (646) | 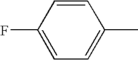 | 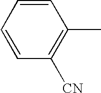 | CH |
| (647) | 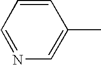 | 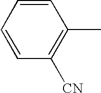 | CH |
| (648) | 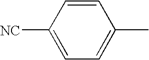 | 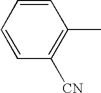 | CH |
| (649) | 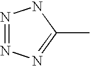 | 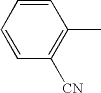 | CH |
| (650) | 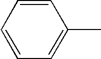 | 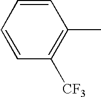 | CH |
| (651) | 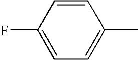 | 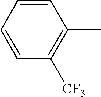 | CH |
| (652) | 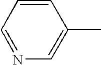 | 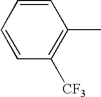 | CH |
| (653) | 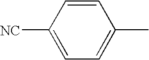 | 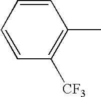 | CH |
| (654) | 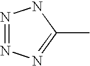 | 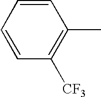 | CH |
| (655) | 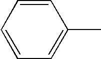 | 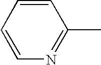 | CH |
| (656) | 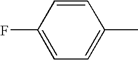 | 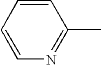 | CH |
| (657) | 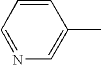 | 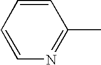 | CH |
| (658) | 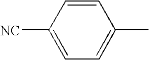 | 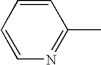 | CH |
| (659) | 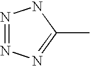 | 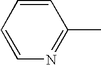 | CH |
| (660) | 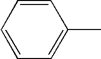 | 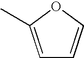 | CH |
| (661) | 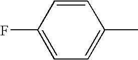 | 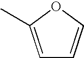 | CH |
| (662) | 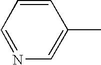 | CF₃ | CH |
| (663) | 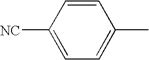 | CF₃ | CH |
| (664) | 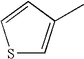 | CF₃ | CH |
| (665) | 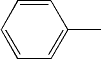 | 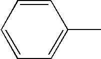 | N |
| (666) | 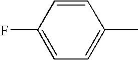 | 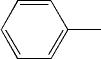 | N |
| (667) | 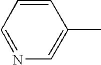 | 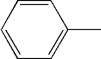 | N |
| (668) | 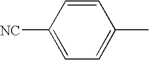 | 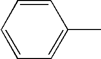 | N |
| (669) | 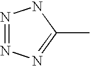 | 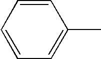 | N |
| (670) | 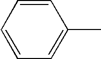 | 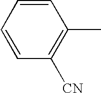 | N |
| (671) | 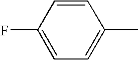 | 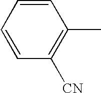 | N |
| (672) | 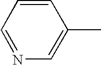 | 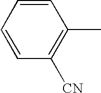 | N |
| (673) | 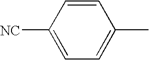 | 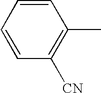 | N |
| (674) | 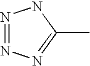 | 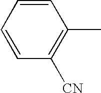 | N |
| (675) | 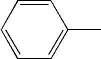 | 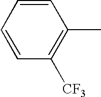 | N |
| (676) | 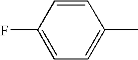 | 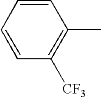 | N |
| (677) | 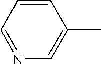 | 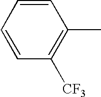 | N |
| (678) | 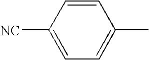 | 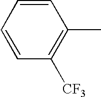 | N |
| (679) | 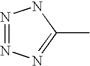 | 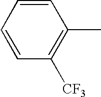 | N |
| (680) | 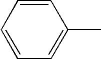 | 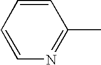 | N |
| (681) | 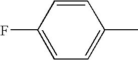 | 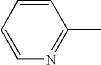 | N |
| (682) | 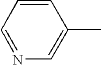 | 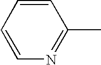 | N |
| (683) | 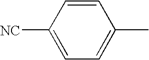 | 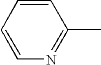 | N |
| (684) | 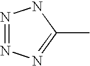 | 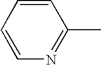 | N |
| (685) | 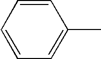 | 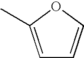 | N |
| (686) | 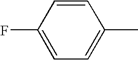 | 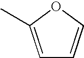 | N |
| (687) | 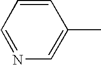 | CF₃ | N |
| (688) | 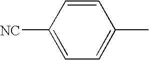 | CF₃ | N |
| (689) | 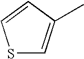 | CF₃ | N |

### EXAMPLES 690-739

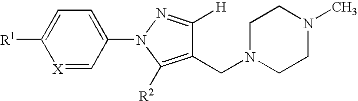

| | R¹ | R² | X |
|---|---|---|---|
| (690) | 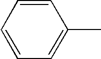 | 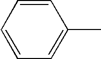 | CH |
| (691) | 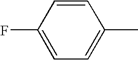 | 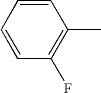 | CH |
| (692) | 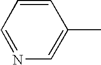 | 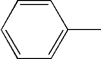 | CH |
| (693) | 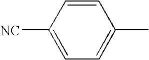 | 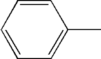 | CH |
| (694) | 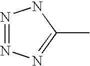 | 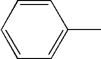 | CH |
| (695) | 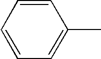 | 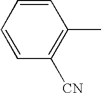 | CH |
| (696) | 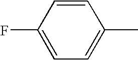 | 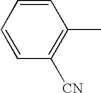 | CH |
| (697) | 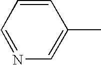 | 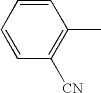 | CH |
| (698) | 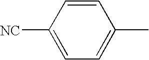 | 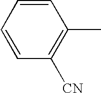 | CH |
| (699) | 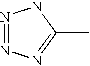 | 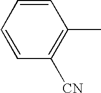 | CH |
| (700) | 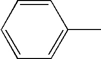 | 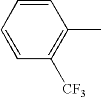 | CH |
| (701) | 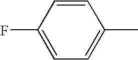 | 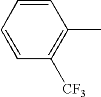 | CH |
| (702) | 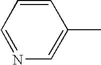 | 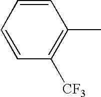 | CH |
| (703) | 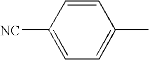 | 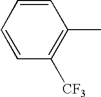 | CH |
| (704) | 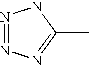 | 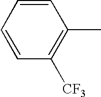 | CH |
| (705) | 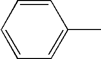 | 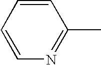 | CH |
| (706) | 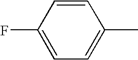 | 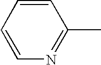 | CH |
| (707) | 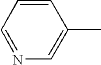 | 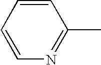 | CH |
| (708) | 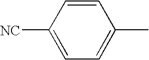 | 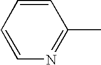 | CH |
| (709) | 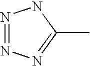 | 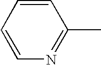 | CH |
| (710) | 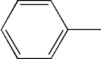 | 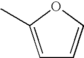 | CH |
| (711) | 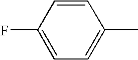 | 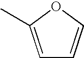 | CH |
| (712) | 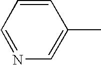 | CF₃ | CH |
| (713) | 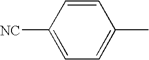 | CF₃ | CH |
| (714) | 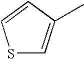 | CF₃ | CH |
| (715) | 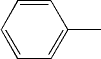 | 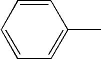 | N |
| (716) | 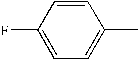 | 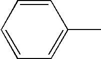 | N |
| (717) | 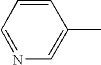 | 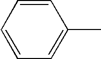 | N |
| (718) | 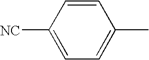 | 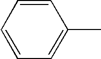 | N |
| (719) | 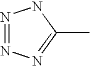 | 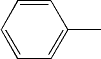 | N |
| (720) | 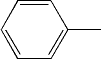 | 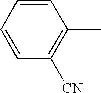 | N |
| (721) | 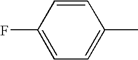 | 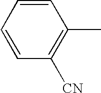 | N |
| (722) | 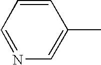 | 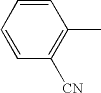 | N |
| (723) | 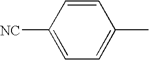 | 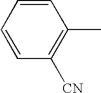 | N |
| (724) | 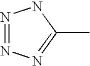 | 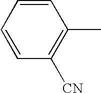 | N |
| (725) | 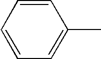 | 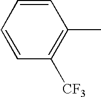 | N |
| (726) | 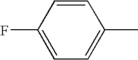 | 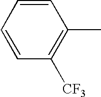 | N |
| (727) | 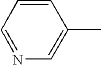 | 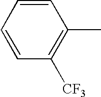 | N |
| (728) | 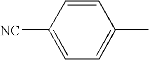 | 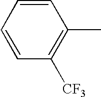 | N |
| (729) | 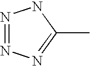 | 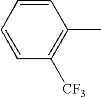 | N |
| (730) | 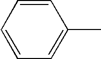 | 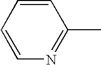 | N |
| (731) | 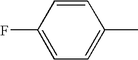 | 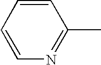 | N |
| (732) | 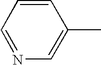 | 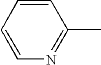 | N |
| (733) | 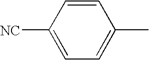 | 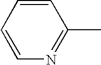 | N |
| (734) | 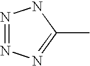 | 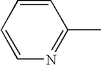 | N |
| (735) | 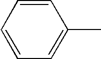 | 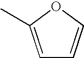 | N |
| (736) | 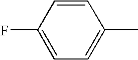 | 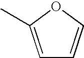 | N |
| (737) | 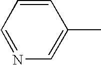 | CF₃ | N |
| (738) | 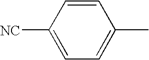 | CF₃ | N |
| (739) | 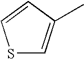 | CF₃ | N |

### EXAMPLES 740-789

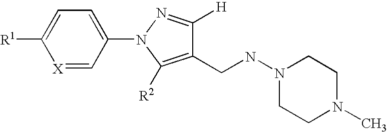

| | R¹ | R² | X |
|---|---|---|---|
| (740) | 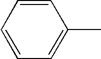 | 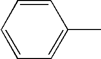 | CH |
| (741) | 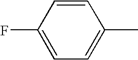 | 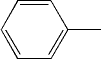 | CH |
| (742) | 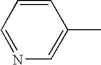 | 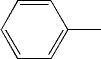 | CH |
| (743) | 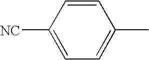 | 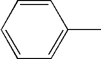 | CH |
| (744) | 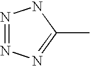 | 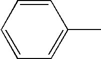 | CH |
| (745) | 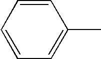 | 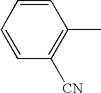 | CH |
| (746) | 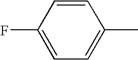 | 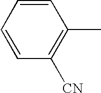 | CH |
| (747) | 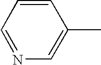 | 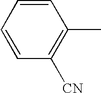 | CH |
| (748) | 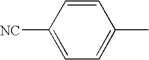 | 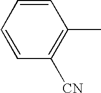 | CH |
| (749) | 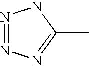 | 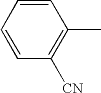 | CH |
| (750) | 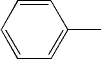 | 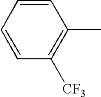 | CH |
| (751) | 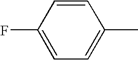 | 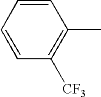 | CH |
| (752) | 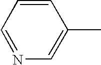 | 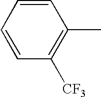 | CH |
| (753) | 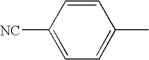 | 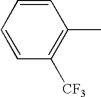 | CH |
| (754) | 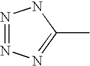 | 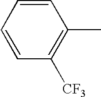 | CH |
| (755) | 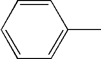 | 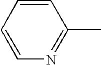 | CH |
| (756) | 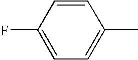 | 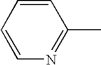 | CH |
| (757) | 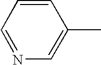 | 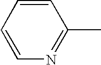 | CH |
| (758) | 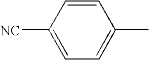 | 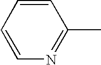 | CH |
| (759) | 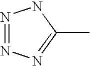 | 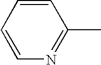 | CH |
| (760) | 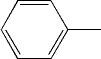 | 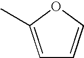 | CH |
| (761) | 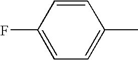 | 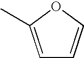 | CH |
| (762) | 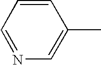 | CF₃ | CH |
| (763) | 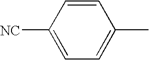 | CF₃ | CH |
| (764) | 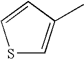 | CF₃ | CH |
| (765) | 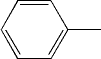 | 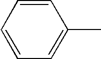 | N |
| (766) | 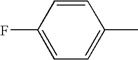 | 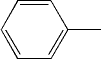 | N |
| (767) | 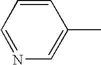 | 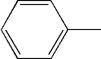 | N |
| (768) | 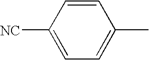 | 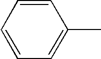 | N |
| (769) | 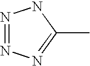 | 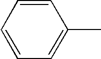 | N |
| (770) | 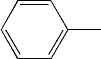 | 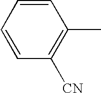 | N |
| (771) | 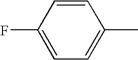 | 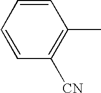 | N |
| (772) | 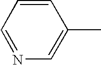 | 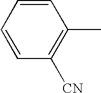 | N |
| (773) | 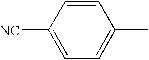 | 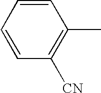 | N |
| (774) | 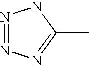 | 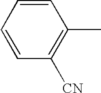 | N |
| (775) | 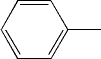 | 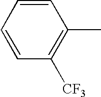 | N |
| (776) | 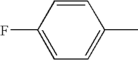 | 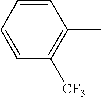 | N |
| (777) | 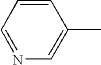 | 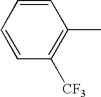 | N |
| (778) | 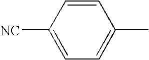 | 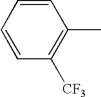 | N |
| (779) | 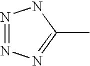 | 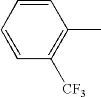 | N |
| (780) | 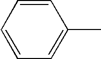 | 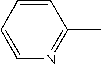 | N |
| (781) | 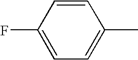 | 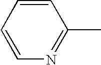 | N |
| (782) | 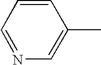 | 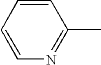 | N |
| (783) | 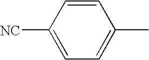 | 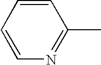 | N |
| (784) | 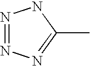 | 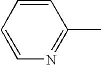 | N |
| (785) | 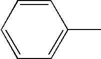 | 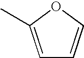 | N |
| (786) | 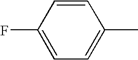 | 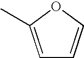 | N |
| (787) | 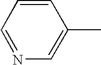 | CF₃ | N |
| (788) | 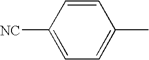 | CF₃ | N |
| (789) | 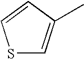 | CF₃ | N |

### EXAMPLES 790-839

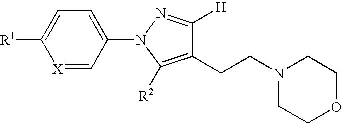

| | R¹ | R² | X |
|---|---|---|---|
| (790) | 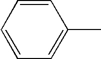 | 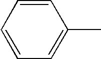 | CH |
| (791) | 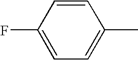 | 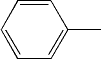 | CH |
| (792) | 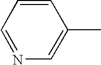 | 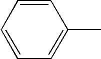 | CH |
| (793) | 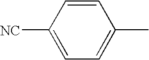 | 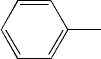 | CH |
| (794) | 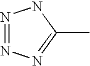 | 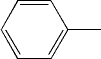 | CH |
| (795) | 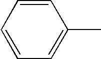 | 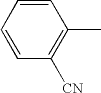 | CH |
| (796) | 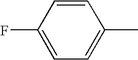 | 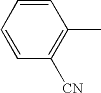 | CH |
| (797) | 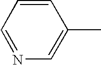 | 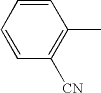 | CH |
| (798) | 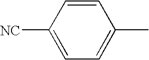 | 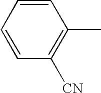 | CH |
| (799) | 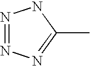 | 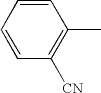 | CH |
| (800) | 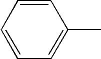 | 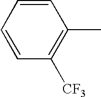 | CH |
| (801) | 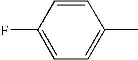 | 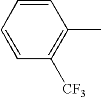 | CH |
| (802) | 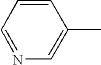 | 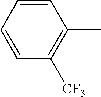 | CH |
| (803) | 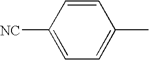 | 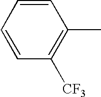 | CH |
| (804) | 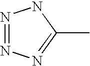 | 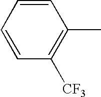 | CH |
| (805) | 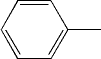 | 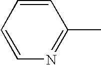 | CH |
| (806) | 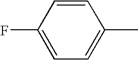 | 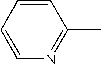 | CH |
| (807) | 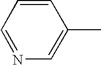 | 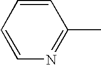 | CH |
| (808) | 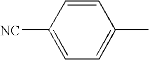 | 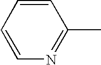 | CH |
| (809) | 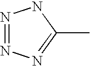 | 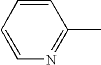 | CH |
| (810) | 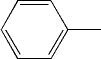 | 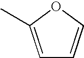 | CH |
| (811) | 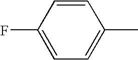 | 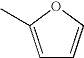 | CH |
| (812) | 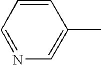 | CF₃ | CH |
| (813) | 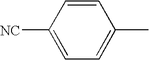 | CF₃ | CH |
| (814) | 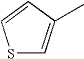 | CF₃ | CH |
| (815) | 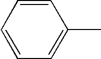 | 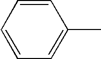 | N |
| (816) | 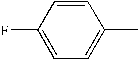 | 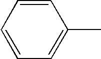 | N |
| (817) | 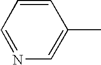 | 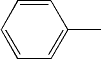 | N |
| (818) | 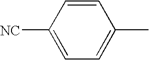 | 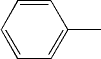 | N |
| (819) | 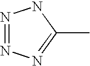 | 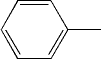 | N |
| (820) | 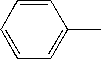 | 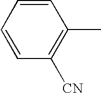 | N |
| (821) | 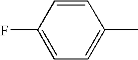 | 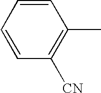 | N |
| (822) | 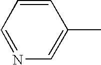 | 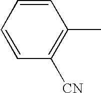 | N |
| (823) | 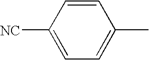 | 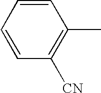 | N |
| (824) | 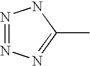 | 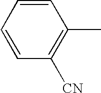 | N |
| (825) | 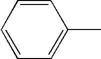 | 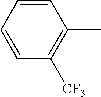 | N |
| (826) | 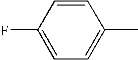 | 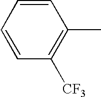 | N |
| (827) | 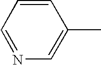 | 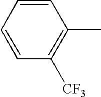 | N |
| (828) | 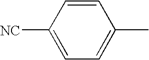 | 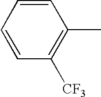 | N |
| (829) | 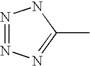 | 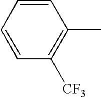 | N |
| (830) | 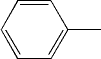 | 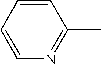 | N |
| (831) | 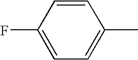 | 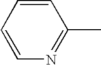 | N |
| (832) | 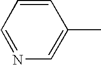 | 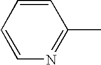 | N |
| (833) | 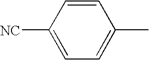 | 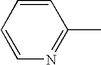 | N |
| (834) | 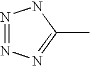 | 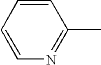 | N |
| (835) | 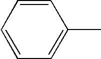 | 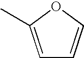 | N |
| (836) | 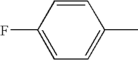 | 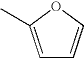 | N |
| (837) | 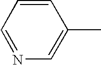 | CF₃ | N |
| (838) | 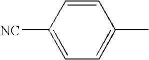 | CF₃ | N |
| (839) | 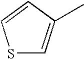 | CF₃ | N |

### EXAMPLES 840-889

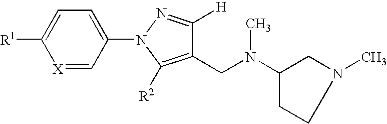

| | R¹ | R² | X |
|---|---|---|---|
| (840) | 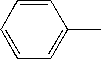 | 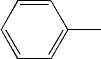 | CH |
| (841) | 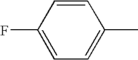 | 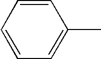 | CH |
| (842) | 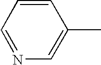 | 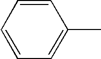 | CH |
| (843) | 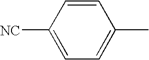 | 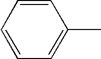 | CH |
| (844) | 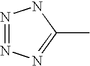 | 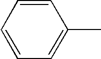 | CH |
| (845) | 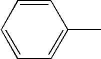 | 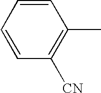 | CH |
| (846) | 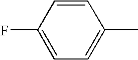 | 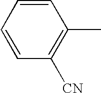 | CH |
| (847) | 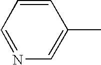 | 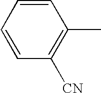 | CH |
| (848) | 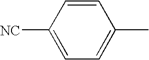 | 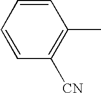 | CH |
| (849) | 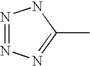 | 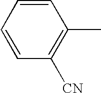 | CH |
| (850) | 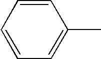 | 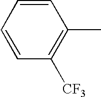 | CH |
| (851) | 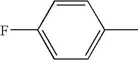 | 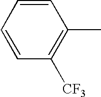 | CH |
| (852) | 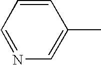 | 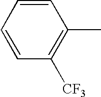 | CH |
| (853) | 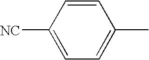 | 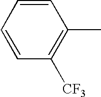 | CH |
| (854) | 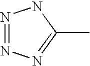 | 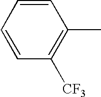 | CH |
| (855) | 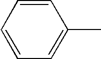 | 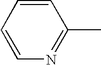 | CH |
| (856) | 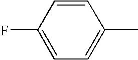 | 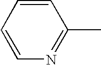 | CH |
| (857) | 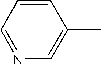 | 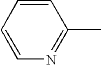 | CH |
| (858) | 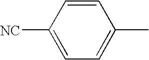 | 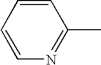 | CH |
| (859) | 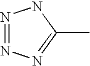 | 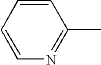 | CH |
| (860) | 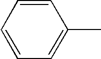 | 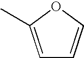 | CH |
| (861) | 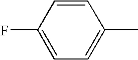 | 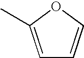 | CH |
| (862) | 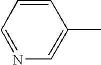 | CF₃ | CH |
| (863) | 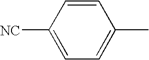 | CF₃ | CH |
| (864) | 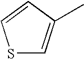 | CF₃ | CH |
| (865) | 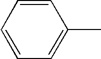 | 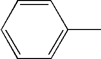 | N |
| (866) | 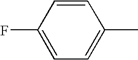 | 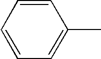 | N |
| (867) | 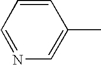 | 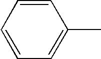 | N |
| (868) | 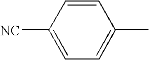 | 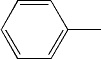 | N |
| (869) | 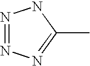 | 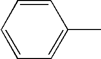 | N |
| (870) | 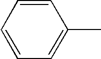 | 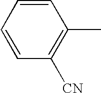 | N |
| (871) | 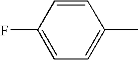 | 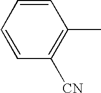 | N |
| (872) | 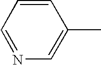 | 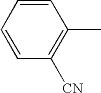 | N |
| (873) | 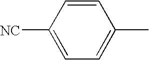 | 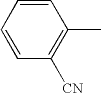 | N |
| (874) | 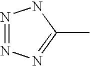 | 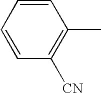 | N |
| (875) | 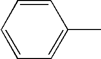 | 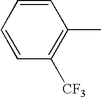 | N |
| (876) | 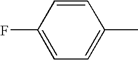 | 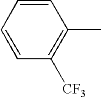 | N |
| (877) | 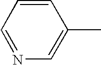 | 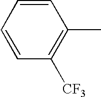 | N |
| (878) | 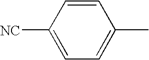 | 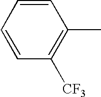 | N |
| (879) | 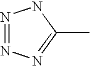 | 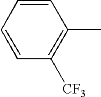 | N |
| (880) | 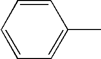 | 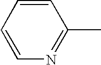 | N |
| (881) | 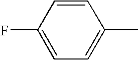 | 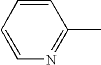 | N |
| (882) | 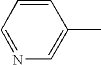 | 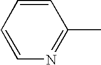 | N |
| (883) | 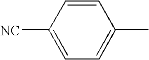 | 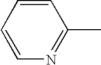 | N |
| (884) | 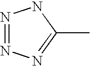 | 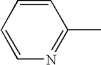 | N |
| (885) | 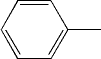 | 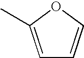 | N |
| (886) | 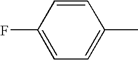 | 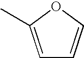 | N |
| (887) | 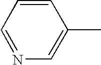 | CF₃ | N |
| (888) | 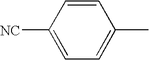 | CF₃ | N |
| (889) | 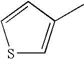 | CF₃ | N |

### EXAMPLES 89-1059

| | | HT2A | HT2C |
|---|---|---|---|
| | | IC50 | IC50 |
| (890) | {2-[1-(4′-Fluorobiphenyl-4-yl)-5-(2- | 1.50E-09 | 2.74E-08 |
| | fluoro-phenyl)-1H-pyrazol-4-yl]ethyl} | | |
| | dimethylamine | | |
| (891) | 1-[5-Furan-2-yl-1-(4-thiophen-3- | 4.50E-09 | 2.10E-07 |
| | ylphenyl)-1H-pyrazol-4-ylmethyl]-4- | | |
| | methylpiperazine | | |
| (892) | 2-{[1-Biphenyl-4-yl-5-(2-fluorophenyl)- | 5.20E-09 | 4.20E-07 |
| | 1H-pyrazol-4-ylmethyl]amino}ethanol | | |
| (893) | 1-[1-Biphenyl-4-yl-5-(2-fluorophenyl)- | 6.40E-09 | 2.30E-07 |
| | 1H-pyrazol-4-ylmethyl]-4-methyl-[1,4] | | |
| | diazepam | | |
| (894) | N-[1-Biphenyl-4-yl-5-(2-fluorophenyl)- | 6.50E-09 | 4.50E-07 |
| | 1H-pyrazol-4-ylmethyl]ethane-1,2- | | |
| | diamine | | |
| (895) | 1-[5-(2-Fluorophenyl)-1-(4′-methoxy- | 7.50E-09 | 1.15E-06 |
| | biphenyl-4-yl)-1H-pyrazol-4-ylmethyl]- | | |
| | 4-methyl-piperazine | | |
| (896) | tert-Butyl 4-[5-(2-fluorophenyl)-1-(4- | 8.00E-09 | 4.30E-05 |
| | trifluoro-methylphenyl)-1H-pyrazol-4- | | |
| | ylmethyl]-piperazine-1-carboxylate | | |
| (897) | [1-Biphenyl-4-yl-5-(2-fluorophenyl)- | 1.10E-08 | 1.00E-06 |
| | 1H-pyrazol-4-ylmethyl]diethylamine | | |
| (898) | 4-{2-[1-Biphenyl-4-yl-5-(2-fluoro- | 1.20E-08 | 1.00E-06 |
| | phenyl)-1H-pyrazol-4-yl]ethyl} | | |
| | morpholine | | |
| (899) | 1-{1-[4-(2,3-Dihydrobenzo[1,4]dioxin- | 1.20E-08 | n.d. |
| | 6-yl)-phenyl]-5-phenyl-1H-pyrazol-4- | | |
| | yl}-4-methyl-piperazine | | |
| (900) | 1-[1-(4′-Fluorobiphenyl-4-yl)-5-phenyl- | 1.30E-08 | 3.10E-07 |
| | 1H-pyrazol-4-ylmethyl]-4- | | |
| | methylpiperazine | | |
| (901) | 1-[1-[4-(2,3-Dihydrobenzo[1,4]dioxin- | 1.30E-08 | 8.70E-07 |
| | 6-yl)-phenyl]-5-(2-fluorophenyl)-1H- | | |
| | pyrazol-4-yl-methyl]-4-methyl- | | |
| | piperazine | | |
| (902) | 1-[1-(4′-Fluorobiphenyl-4-yl)-5-(2- | 1.31E-08 | 2.15E-07 |
| | fluoro-phenyl)-1H-pyrazol-4-ylmethyl]- | | |
| | 4-methyl-piperazine | | |
| (903) | [1-Biphenyl-4-yl-5-(2-fluorophenyl)- | 1.40E-08 | 4.70E-07 |
| | 1H-pyrazol-4-ylmethyl]-(4- | | |
| | methylpiperazin-1-yl)amine | | |
| (904) | 1-[1-Biphenyl-4-yl-5-(2-fluorophenyl)- | 1.40E-08 | 2.00E-06 |
| | 1H-pyrazol-4-ylmethyl]pyrrolidin-3-ol | | |
| (905) | [1-Biphenyl-4-yl-5-(2-fluorophenyl)- | 1.60E-08 | 1.00E-06 |
| | 1H-pyrazol-4-ylmethyl]-(2- | | |
| | methoxyethyl)amine | | |
| (906) | 2-{[1-Biphenyl-4-yl-5-(2-fluorophenyl)- | 1.60E-08 | 1.00E-06 |
| | 1H-pyrazol-4-ylmethyl]methylamino} | | |
| | ethanol | | |
| (907) | [1-Biphenyl-4-yl-5-(2-fluorophenyl)- | 1.60E-08 | 8.40E-08 |
| | 1H-pyrazol-4-ylmethyl]methyl-(1- | | |
| | methylpyrrolidin-3-yl)amine | | |
| (908) | 4-{3-[1-Biphenyl-4-yl-5-(2- | 1.70E-08 | n.d. |
| | fluorophenyl)-1H-pyrazol-4-yl]allyl} | | |
| | morpholine | | |
| (909) | 1-[5-(2-Fluorophenyl)-1-(4-pyrrol-1- | 1.70E-08 | 2.10E-07 |
| | ylphenyl)-1H-pyrazol-4-ylmethyl]-4- | | |
| | methylpiperazine | | |
| (910) | 1-[1-(4′-Methoxybiphenyl-4-yl)-5- | 1.80E-08 | n.d. |
| | phenyl-1H-pyrazol-4-yl]-4- | | |
| | methylpiperazine | | |
| (911) | 1-[1-(4′-Fluorobiphenyl-4-yl)-5-(3- | 1.90E-08 | n.d. |
| | fluoro-phenyl)-1H-pyrazol-4-ylmethyl]- | | |
| | 4-methyl-piperazine | | |
| (912) | N-[1-Biphenyl-4-yl-5-(2-fluorophenyl)- | 2.00E-08 | 9.20E-07 |
| | 1H-pyrazol-4-ylmethyl]-N,N′,N′- | | |
| | trimethylethane-1,2-diamine | | |
| (913) | 1-{2-[1-(4′-Fluorobiphenyl-4-yl)-5-(2- | 2.00E-08 | 6.20E-07 |
| | fluoro-phenyl)-1H-pyrazol-4-yl]ethyl} | | |
| | pyrrolidin-3-ol | | |
| (914) | [1-Biphenyl-4-yl-5-(2-fluorophenyl)- | 2.10E-08 | 4.50E-07 |
| | 1H-pyrazol-4-ylmethyl]dimethylamine | | |
| (915) | C-(1-Biphenyl-4-yl-5-phenyl-1H- | 2.10E-08 | 9.20E-07 |
| | pyrazol-4-yl)-methylamine | | |
| (916) | 1-[5-(2-Fluorophenyl)-1-(4′- | 2.20E-08 | 9.60E-07 |
| | methylbiphenyl-4-yl)-1H-pyrazol-4- | | |
| | ylmethyl]-4-methylpiperazine | | |
| (917) | 4-{3-[1-Biphenyl-4-yl-5-(2-fluoro- | 2.30E-08 | n.d. |
| | phenyl)-1H-pyrazol-4-yl]propyl} | | |
| | morpholine | | |
| (918) | (1-Biphenyl-4-yl-5-phenyl-1H-pyrazol- | 2.30E-08 | 1.00E-06 |
| | 4-yl-methyl)pyridin-2-ylmethylamine | | |
| (919) | 1-[2-(2,4-Difluorophenyl)ethyl]-4-[5-(2- | 2.30E-08 | 1.00E-06 |
| | fluoro-phenyl)-1-pyridin-2-yl-1H- | | |
| | pyrazol-4-ylmethyl]-piperazine | | |
| (920) | 1-[1-(4′-Chlorobiphenyl-4-yl)-5-(2- | 2.30E-08 | 3.30E-07 |
| | fluoro-phenyl)-1H-pyrazol-4-ylmethyl]- | | |
| | 4-methyl-piperazine | | |
| (921) | 1-{2-[1-(4′-Fluorobiphenyl-4-yl)-5- | 2.33E-08 | 7.30E-07 |
| | (2-fluoro-phenyl)-1H-pyrazol-4-yl] | | |
| | ethyl}-4-methylpiperazine | | |
| (922) | 1-[5-(2-Fluorophenyl)-1-(5-phenyl- | 2.40E-08 | 6.60E-07 |
| | pyridin-2-yl)-1H-pyrazol-4-ylmethyl]- | | |
| | 4-methylpiperazine | | |
| (923) | 1-[1-(4′-Fluorobiphenyl-4-yl)-5-(4- | 2.50E-08 | 7.50E-07 |
| | fluoro-phenyl)-1H-pyrazol-4-ylmethyl]- | | |
| | 4-methylpiperazine | | |
| (924) | 1-[1-Biphenyl-4-yl-5-(2-fluorophenyl)- | 2.60E-08 | 6.60E-07 |
| | 1H-pyrazol-4-ylmethyl]piperazine | | |
| (925) | (1-Biphenyl-4-yl-5-phenyl-1H-pyrazol- | 2.60E-08 | 5.20E-07 |
| | 4-yl-methyl)-(1-methyl-1H-pyrrol-2- | | |
| | ylmethyl)amine | | |
| (926) | 1-[1-Biphenyl-4-yl-5-(2-fluoro- | 2.73E-08 | 6.00E-07 |
| | phenyl)-1H-pyrazol-4-ylmethyl]-4- | | |
| | methylpiperazine | | |
| (927) | 1-[1-Biphenyl-4-yl-5-(2-fluoro- | 2.80E-08 | 1.00E-06 |
| | phenyl)-1H-pyrazol-4-ylmethyl]-4- | | |
| | ethylpiperazine | | |
| (928) | 1-Ethyl-4-{2-[1-(4′-fluorobiphenyl-4- | 2.80E-08 | 1.30E-06 |
| | yl)-5-(2-fluorophenyl)-1H-pyrazol-4-yl] | | |
| | ethyl}piperazine | | |
| (929) | N-{1-[1-Biphenyl-4-yl-5-(2-fluoro- | 2.90E-08 | 1.00E-06 |
| | phenyl)-1H-pyrazol-4-ylmethyl] | | |
| | pyrrolidin-3-yl}acetamide | | |
| (930) | 1-[5-(2-Fluorophenyl)-1-(3′-methoxy- | 2.90E-08 | 6.90E-07 |
| | biphenyl-4-yl)-1H-pyrazol-4-ylmethyl]- | | |
| | 4-methyl-piperazine | | |
| (931) | 1-[1-Biphenyl-4-yl-5-(2-fluorophenyl)- | 3.00E-08 | 1.00E-06 |
| | 1H-pyrazol-4-ylmethyl]piperidine | | |
| (932) | (1-Biphenyl-4-yl-5-phenyl-1H- | 3.10E-08 | 1.00E-06 |
| | pyrazol-4-yl-methyl)pyridin-4- | | |
| | ylmethylamine | | |
| (933) | [5-(4-Chlorophenyl)-1-(4′-fluoro- | 3.10E-08 | 1.00E-06 |
| | biphenyl-4-yl)-1H-pyrazol-4-yl] | | |
| | methanol | | |
| (934) | 1-Biphenyl-4-yl-5-(2-fluorophenyl)- | 3.20E-08 | 1.00E-06 |
| | 4-pyrrolidin-1-ylmethyl-1H-pyrazole | | |
| (935) | [1-Biphenyl-4-yl-5-(2-fluorophenyl)- | 3.40E-08 | 1.00E-06 |
| | 1H-pyrazol-4-ylmethyl]-(3- | | |
| | methoxypropyl)amine | | |
| (936) | {1-[1-Biphenyl-4-yl-5-(2-fluoro- | 3.50E-08 | n.d. |
| | phenyl)-1H-pyrazol-4-ylmethyl] | | |
| | pyrrolidin-3-yl}dimethyl- | | |
| | amine | | |
| (937) | [5-(2-Fluorophenyl)-1-(5-trifluoro- | 3.50E-08 | 1.00E-06 |
| | methylpyridin-2-yl)-1H-pyrazol-4- | | |
| | ylmethyl]dimethylamine | | |
| (938) | 1-Ethyl-4-[1-(4′-fluorobiphenyl-4-yl)- | 3.50E-08 | n.d. |
| | 5-(3-methoxyphenyl)-1H-pyrazol- | | |
| | 4-ylmethyl]-piperazine | | |
| (939) | 1-Biphenyl-4-yl-4-(2,5-dihydropyrrol- | 3.60E-08 | n.d. |
| | 1-yl-methyl)-5-(2-fluorophenyl)-1H- | | |
| | pyrazole | | |
| (940) | 1-[1-(4′-Fluorobiphenyl-4-yl)-5-m- | 3.70E-08 | n .d. |
| | tolyl-1H-pyrazol-4-ylmethyl]- | | |
| | 4-methylpiperazine | | |
| (941) | 1-[5-(2-Fluorophenyl)-1-(5-trifluoro- | 3.90E-08 | 1.00E-06 |
| | methylpyridin-2-yl)-1H-pyrazol-4- | | |
| | ylmethyl]-4-methyl-piperazine | | |
| (942) | (1-Biphenyl-4-yl-5-phenyl-1H-pyrazol- | 3.90E-08 | 1.00E-06 |
| | 4-yl-methyl)-(1-methyl-1H-imidazol- | | |
| | 2-ylmethyl)-amine | | |
| (943) | 1-[5-(3-Chlorophenyl)-1-(4′- | 4.10E-08 | 1.00E-06 |
| | fluorobiphenyl-4-yl)-1H-pyrazol-4- | | |
| | ylmethyl]-4-isopropylpiperazine | | |
| (944) | 1-[5-(2-Fluorophenyl)-1-(4-trifluoro- | 4.30E-08 | 7.90E-07 |
| | methyl-phenyl)-1H-pyrazol-4- | | |
| | ylmethyl]piperazine | | |
| (945) | 4-[1-Biphenyl-4-yl-5-(2-fluorophenyl)- | 4.30E-08 | 1.00E-06 |
| | 1H-pyrazol-4-ylmethyl] | | |
| | thiomorpholine 1,1-dioxide | | |
| (946) | N-[5-(2-chlorophenyl)-1-(4′-fluoro- | 4.40E-08 | 4.90E-07 |
| | biphenyl-4-yl)-1H-pyrazol-4-ylmethyl]- | | |
| | N,N′,N′-trimethyl- | | |
| | ethane-1,2-diamine | | |
| (947) | [1-Biphenyl-4-yl-5-(2-fluorophenyl)- | 4.69E-08 | 1.00E-06 |
| | 1H-pyrazol-4-ylmethyl]-(1-ethyl- | | |
| | pyrrolidin-2-yl-methyl)amine | | |
| (948) | 1-[1-Biphenyl-4-yl-5-(2-fluorophenyl)- | 4.80E-08 | n.d. |
| | 1H-pyrazol-4-ylmethyl]azepan | | |
| (949) | 1-(1-Biphenyl-4-yl-5-pyridin-2-yl- | 5.00E-08 | 1.00E-06 |
| | 1H-pyrazol-4-ylmethyl)-4- | | |
| | methylpiperazine | | |
| (950) | 1-[2-(4-Fluorophenyl)ethyl]-4-[5-(2- | 5.30E-08 | n.d. |
| | fluoro-phenyl)-1-pyridin-2-yl-1H- | | |
| | pyrazol-4-ylmethyl]-piperazine | | |
| (951) | [5-(2-Chlorophenyl)-1-(4′-fluoro- | 5.30E-08 | 1.00E-06 |
| | biphenyl-4-yl)-1H-pyrazol-4-ylmethyl] | | |
| | diethylamine | | |
| (952) | 4′-[5-(2-Fluorophenyl)-4-(4-methyl- | 5.30E-08 | 7.90E-07 |
| | piperazin-1-ylmethyl)pyrazol-1-yl] | | |
| | biphenyl-4-carbonitrile | | |
| (953) | 1-[1-(2′,5′-Difluorobiphenyl-4-yl)-5- | 5.50E-08 | 4.70E-07 |
| | (2-fluoro-phenyl)-1H-pyrazol-4- | | |
| | ylmethyl]-4-methyl-piperazine | | |
| (954) | 1-[1-(4′-Fluorobiphenyl-4-yl)-5-(4- | 5.60E-08 | n.d. |
| | methoxy-phenyl)-1H-pyrazol-4- | | |
| | ylmethyl]-4-(2-pyrroli- | | |
| | din-1-ylethyl)piperazine | | |
| (955) | 1-[1-(2′,4′-Difluorobiphenyl-4-yl)-5- | 5.60E-08 | n.d. |
| | (2-fluoro-phenyl)-1H-pyrazol-4- | | |
| | ylmethyl]-4-methyl-piperazine | | |
| (956) | 1-[1-(4′-Ethylbiphenyl-4-yl)-5-(2- | 5.70E-08 | n.d. |
| | fluorophenyl)-1H-pyrazol-4-ylmethyl]- | | |
| | 4-methylpiperazine | | |
| (957) | Ethyl 4-[1-biphenyl-4-yl-5-(2-fluoro- | 5.80E-08 | n.d. |
| | phenyl)-1H-pyrazol-4-ylmethyl] | | |
| | piperazine-1-carboxylate | | |
| (958) | 1-[5-(2-Fluorophenyl)-1-(4′- | 5.81E-08 | 8.30E-07 |
| | isopropylbiphenyl-4-yl)-1H-pyrazol- | | |
| | 4-ylmethyl]-4-methyl-piperazine | | |
| (959) | 1-[1-(2′,3′-Difluoro-4′-methylbiphenyl- | 6.00E-08 | n.d. |
| | 4-yl)-5-(2-fluorophenyl)-1H-pyrazol-4- | | |
| | ylmethyl]-4-methylpiperazine | | |
| (960) | 1-[1-(3′,4′-Difluorobiphenyl-4-yl)-5-(2- | 6.00E-08 | 3.40E-07 |
| | fluoro-phenyl)-1H-pyrazol-4-ylmethyl]- | | |
| | 4-methyl-piperazine | | |
| (961) | 1-(3-{[1-Biphenyl-4-yl-5-(2-fluoro- | 6.40E-08 | 1.00E-06 |
| | phenyl)-1H-pyrazol-4-ylmethyl]amino} | | |
| | propyl)pyrrolidin-2-one | | |
| (962) | 3-{1-[1-Biphenyl-4-yl-5-(2-fluoro- | 6.60E-08 | n.d. |
| | phenyl)-1H-pyrazol-4-ylmethyl} | | |
| | piperidin-4-ylmethyl]pyridine | | |
| (963) | 1-[1-(3′-Chlorobiphenyl-4-yl)-5-(2- | 6.90E-08 | n.d. |
| | fluoro-phenyl)-1H-pyrazol-4-ylmethyl]- | | |
| | 4-methyl-piperazine | | |
| (964) | 2-[1-Biphenyl-4-yl-5-(2-fluoro- | 7.00E-08 | 1.00E-06 |
| | phenyl)-1H-pyrazol-4-ylmethyl]- | | |
| | 1,2,3,4-tetrahydro-iso-quinoline | | |
| (965) | 1-[5-(2-Chlorophenyl)-1-(4′-fluoro- | 7.20E-08 | 6.00E-07 |
| | biphenyl-4-yl)-1H-pyrazol-4-ylmethyl]- | | |
| | 4-methylpiperazine | | |
| (966) | 1-[1-(4′-Fluorobiphenyl-4-yl)-5-o-tolyl- | 7.20E-08 | n.d. |
| | 1H-pyrazol-4-ylmethyl]-4-methyl- | | |
| | piperazine | | |
| (967) | 1-[1-(2′,3′-Difluorobiphenyl-4-yl)-5- | 7.30E-08 | n.d. |
| | (2-fluoro-phenyl)-1H-pyrazol-4- | | |
| | ylmethyl]-4-methyl-piperazine | | |
| (968) | 1-[1-Biphenyl-4-yl-5-(2-fluorophenyl)- | 7.50E-08 | n.d. |
| | 1H-pyrazol-4-ylmethyl]-4-pyridin- | | |
| | 4-ylmethylpiperazine | | |
| (969) | (1H-Benzoimidazol-2-ylmethyl)-[1- | 7.60E-08 | 1.00E-06 |
| | biphenyl-4-yl-5-(2-fluorophenyl)-1H- | | |
| | pyrazol-4-ylmethyl]-amine | | |
| (970) | {4′-[5-(2-Fluorophenyl)-4-(4-methyl- | 8.10E-08 | n.d. |
| | piperazin-1-ylmethyl)pyrazol-1-yl] | | |
| | biphenyl-2-ylmethyl}-dimethylamine | | |
| (971) | tert-Butyl 4-[1-biphenyl-4-yl-5-(2- | 8.20E-08 | 1.00E-06 |
| | fluoro-phenyl)-1H-pyrazol-4-ylmethyl] | | |
| | piperazine-1-carboxylate | | |
| (972) | 2-[2-(4′-Fluorobiphenyl-4-yl)-4-(4- | 8.50E-08 | n.d. |
| | methyl-piperazin-1-ylmethyl)-2H- | | |
| | pyrazol-3-yl]-pyrazine | | |
| (973) | 1-[1-(3′,5′-Dichlorobiphenyl-4-yl)-5-(2- | 8.60E-08 | n.d. |
| | fluoro-phenyl)-1H-pyrazol-4-ylmethyl]- | | |
| | 4-methyl-piperazine | | |
| (974) | [1-(4′-Fluorobiphenyl-4-yl)-5-phenyl- | 8.70E-08 | n.d. |
| | 1H-pyrazol-4-ylmethyl]-(4-methyl- | | |
| | piperazin-1-yl)amine | | |
| (975) | 1-[5-(2-Chlorophenyl)-1-(4′-fluoro- | 8.80E-08 | 1.00E-06 |
| | biphenyl-4-yl)-1H-pyrazol-4-ylmethyl]- | | |
| | 4-ethylpiperazine | | |
| (976) | 1-[1-(2′-Fluorobiphenyl-4-yl)-5-(2- | 9.30E-08 | 3.71E-07 |
| | fluoro-phenyl)-1H-pyrazol-4-ylmethyl]- | | |
| | 4-methyl-piperazine | | |
| (977) | 2-[1-Biphenyl-4-yl-5-(2-fluorophenyl)- | 9.40E-08 | 6.80E-07 |
| | 1H-pyrazol-4-ylmethyl]-5-methyl-2,5- | | |
| | diazabicyclo-[2.2.1]heptane | | |
| (978) | 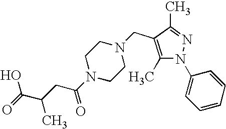 | 1.00E-07 | n.d. |
| (979) | 1-[1-Biphenyl-4-yl-5-(2-fluorophenyl)- | 1.00E-07 | n.d. |
| | 1H-pyrazol-4-ylmethyl]-3,5- | | |
| | dimethylpiperazine | | |
| (980) | 2-{[1-Biphenyl-4-yl-5-(2-fluorophenyl)- | 1.00E-07 | n.d. |
| | 1H-pyrazol-4-ylmethyl]amino}-N- | | |
| | (4-nitrophenyl)-acetamide | | |
| (981) | 1-[1-(2′,5′-Difluorobiphenyl-4-yl)-5- | 1.10E-07 | 1.00E-06 |
| | phenyl-1H-pyrazol-4-ylmethyl]- | | |
| | 4-methylpiperazine | | |
| (982) | Cyclopropylbis-[1-(4′-fluorobiphenyl-4- | 1.10E-07 | n.d. |
| | yl)-5-furan-2-yl-1H-pyrazol-4- | | |
| | ylmethyl]amine | | |
| (983) | 1-[5-(2-Fluorophenyl)-1-(4- | 1.20E-07 | 5.20E-07 |
| | isopropylphenyl)-1H-pyrazol-4- | | |
| | ylmethyl]-4-methylpiperazine | | |
| (984) | 1-[1-(2′-Chlorobiphenyl-4-yl)-5-(2- | 1.20E-07 | n.d. |
| | fluoro-phenyl)-1H-pyrazol-4-ylmethyl]- | | |
| | 4-methyl-piperazine | | |
| (985) | 2-{4-[1-Biphenyl-4-yl-5-(2-fluoro- | 1.22E-07 | n.d. |
| | phenyl)-1H-pyrazol-4-ylmethyl] | | |
| | piperazin-1-yl}-1-pyrrolidin- | | |
| | 1-ylethanone | | |
| (986) | 1-[1-Biphenyl-4-yl-5-(2-fluorophenyl)- | 1.30E-07 | n.d. |
| | 1H-pyrazol-4-ylmethyl]-4- | | |
| | methylpiperidine | | |
| (987) | 2-{[1-Biphenyl-4-yl-5-(2-fluorophenyl)- | 1.30E-07 | n.d. |
| | 1H-pyrazol-4-ylmethyl]amino}-2- | | |
| | cyanoacetamide | | |
| (988) | N-{4′-[5-(2-Fluorophenyl)-4-(4-methyl- | 1.30E-07 | n .d. |
| | piperazin-1-ylmethyl)pyrazol-1-yl] | | |
| | biphenyl-3-yl}acetamide | | |
| (989) | 1-[1-(4-Bromophenyl)-5-(2-fluoro- | 1.30E-07 | n.d. |
| | phenyl)-1H-pyrazol-4-ylmethyl]-4- | | |
| | methylpiperazine | | |
| (990) | 1-[5-(2-Fluorophenyl)-1-(4-trifluoro- | 1.33E-07 | 4.90E-07 |
| | methyl-phenyl)-1H-pyrazol-4- | | |
| | ylmethyl]-4-methyl-piperazine | | |
| (991) | (1-Azabicyclo[2.2.2]oct-3-yl)-[1- | 1.40E-07 | n.d. |
| | biphenyl-4-yl-5-(2-fluorophenyl)-1H- | | |
| | pyrazol-4-ylmethyl]-amine | | |
| (992) | [5-(3-Chlorophenyl)-1-(4′-fluoro- | 1.40E-07 | n.d. |
| | biphenyl-4-yl)-1H-pyrazol-4- | | |
| | yl]methanol | | |
| (993) | 4′-[5-(2-Fluorophenyl)-4-(4- | 1.40E-07 | n.d. |
| | methylpiperazin-1-ylmethyl)pyrazol-1- | | |
| | yl]biphenyl-3-carbonitrile | | |
| (994) | 1-[1-(4′-Fluorobiphenyl-4-yl)-5-p- | 1.40E-07 | n.d. |
| | tolyl-1H-pyrazol-4-ylmethyl]-4- | | |
| | methylpiperazine | | |
| (995) | [1-Biphenyl-4-yl-5-(2-fluorophenyl)- | 1.60E-07 | n.d. |
| | 1H-pyrazol-4-ylmethyl]methyl-(1- | | |
| | methylpiperidin-4-yl)amine | | |
| (996) | 5-[1-Biphenyl-4-yl-5-(2-fluorophenyl)- | 1.60E-07 | n.d. |
| | 1H-pyrazol-4-ylmethyl]-2-oxa-5- | | |
| | azabicyclo-[2.2.1]heptane | | |
| (997) | 1-[5-(2-Chlorophenyl)-1-(4′-fluoro- | 1.60E-07 | 1.00E-06 |
| | biphenyl-4-yl)-1H-pyrazol-4-ylmethyl]- | | |
| | 4-isopropylpiperazine | | |
| (998) | 1-[1-Biphenyl-4-yl-5-(2-fluorophenyl)- | 1.70E-07 | n.d. |
| | 1H-pyrazol-4-ylmethyl]-1,2,3,6- | | |
| | tetrahydro-pyridine | | |
| (999) | Ethyl 4-{[1-biphenyl-4-yl-5-(2- | 1.70E-07 | n.d. |
| | fluorophenyl)-1H-pyrazol-4-ylmethyl] | | |
| | methylamino}piperidine-1-carboxylate | | |
| (1000) | 1-[5-(2-Fluorophenyl)-1-(4-pyridin-3- | 1.70E-07 | n.d. |
| | ylphenyl)-1H-pyrazol-4-ylmethyl]- | | |
| | 4-methylpiperazine | | |
| (1001) | [1-Biphenyl-4-yl-5-(2-fluorophenyl)- | 1.70E-07 | n.d. |
| | 1H-pyrazol-4-ylmethyl]-(3-imidazol- | | |
| | 1-ylpropyl)amine | | |
| (1002) | [1-Biphenyl-4-yl-5-(2-fluorophenyl)- | 1.70E-07 | n.d. |
| | 1H-pyrazol-4-ylmethyl]-[2-(1H- | | |
| | imidazol-4-yl)-ethyl]amine | | |
| (1003) | 1-[5-(4-Chlorophenyl)-1-(4′-fluoro- | 1.70E-07 | 1.00E-06 |
| | biphenyl-4-yl)-1H-pyrazol-4-ylmethyl] | | |
| | pyrrolidin-3-ol | | |
| (1004) | [1-Biphenyl-4-yl-5-(2-fluorophenyl)- | 1.80E-07 | n.d. |
| | 1H-pyrazol-4-ylmethyl]pyridin-3- | | |
| | ylmethylamine | | |
| (1005) | [1-(2′,5′-Difluorobiphenyl-4-yl)-5-(2- | 1.80E-07 | n.d. |
| | fluoro-phenyl)-1H-pyrazol-4-ylmethyl]- | | |
| | (1H-pyrazol-3-yl)amine | | |
| (1006) | N3-[1-Biphenyl-4-yl-5-(2-fluoro- | 1.80E-07 | n.d. |
| | phenyl)-1H-pyrazol-4-ylmethyl] | | |
| | pyridine-3,4-diamine | | |
| (1007) | 1-[5-(3,4-Dichlorophenyl)-1-(4′-fluoro- | 1.90E-07 | n.d. |
| | biphenyl-4-yl)-1H-pyrazol-4-ylmethyl]- | | |
| | 4-methyl-piperazine | | |
| (1008) | {1-[1-Biphenyl-4-yl-5-(2-fluorophenyl)- | 2.00E-07 | n.d. |
| | 1H-pyrazol-4-ylmethyl]piperidin-4-yl} | | |
| | dimethyl-amine | | |
| (1009) | 1-(1-Biphenyl-4-yl-5-pyridin-2-yl-1H- | 2.00E-07 | n.d. |
| | pyrazol-4-ylmethyl)piperazine | | |
| (1010) | 2-{[1-Biphenyl-4-yl-5-(2-fluorophenyl)- | 2.00E-07 | n.d. |
| | 1H-pyrazol-4-ylmethyl]amino}- | | |
| | N,N-dimethyl-acetamide | | |
| (1011) | 1-Ethyl-4-[1-(4′-fluorobiphenyl-4-yl)- | 2.00E-07 | n.d. |
| | 5-(4-methoxyphenyl)-1H-pyrazol-4- | | |
| | ylmethyl]-piperazine | | |
| (1012) | 1-[1-Biphenyl-4-yl-5-(2-fluorophenyl)- | 2.00E-07 | n.d. |
| | 1H-pyrazol-4-ylmethyl]-1H-pyridin- | | |
| | 2-one | | |
| (1013) | 1-[1-(2′-Fluorobiphenyl-4-yl)-5-(4- | 2.10E-07 | 1.00E-06 |
| | fluoro-phenyl)-1H-pyrazol-4-ylmethyl]- | | |
| | 4-methyl-piperazine | | |
| (1014) | Methyl 3-{[1-biphenyl-4-yl-5-(2-fluoro- | 2.10E-07 | n.d. |
| | phenyl)-1H-pyrazol-4-ylmethyl]amino} | | |
| | propionate | | |
| (1015) | 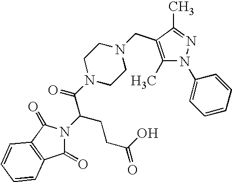 | 2.20E-07 | n.d. |
| (1016) | [5-(4-Chlorophenyl)-1-(4′-fluoro- | 2.20E-07 | 1.00E-06 |
| | biphenyl-4-yl)-1H-pyrazol-4-ylmethyl] | | |
| | diethylamine | | |
| (1017) | 1-[5-(2-Fluorophenyl)-1-(3′- | 2.20E-07 | n.d. |
| | methylbiphenyl-4-yl)-1H-pyrazol-4- | | |
| | ylmethyl]-4-methylpiperazine | | |
| (1018) | 2-{4-[1-Biphenyl-4-yl-5-(2-fluoro- | 2.30E-07 | n.d. |
| | phenyl)-1H-pyrazol-4-ylmethyl] | | |
| | piperazin-1-yl}-N-ethyl- | | |
| | acetamide | | |
| (1019) | 1-{1-{5-(2-Chlorophenyl)-1-(4′-fluoro- | 2.30E-07 | 4.00E-08 |
| | biphenyl-4-yl)-1H-pyrazol-4-ylmethyl] | | |
| | piperidin-4-yl}-1- | | |
| | (4-fluorophenyl)methanone | | |
| (1020) | 1-[5-(2-Fluorophenyl)-1-(2′-methyl- | 2.30E-07 | n.d. |
| | biphenyl-4-yl)-1H-pyrazol-4-ylmethyl]- | | |
| | 4-methylpiperazine | | |
| (1021) | 1-[1-(2′,5′-Difluorobiphenyl-4-yl)-5-(4- | 2.40E-07 | 1.00E-06 |
| | fluoro-phenyl)-1H-pyrazol-4-ylmethyl]- | | |
| | 4-methyl-piperazine | | |
| (1022) | [1-Biphenyl-4-yl-5-(2-fluorophenyl)- | 2.40E-07 | 1.00E-06 |
| | 1H-pyrazol-4-ylmethyl] | | |
| | isoxazol-3-ylamine | | |
| (1023) | (1-Biphenyl-4-yl-5-phenyl-1H-pyrazol- | 2.40E-07 | n.d. |
| | 4-yl-methyl)-(5-methylisoxazol-3- | | |
| | ylmethyl)amine | | |
| (1024) | N-[5-(4-Chlorophenyl)-1-(4′-fluoro- | 2.40E-07 | 1.00E-06 |
| | biphenyl-4-yl)-1H-pyrazol-4-ylmethyl]- | | |
| | N,N′,N′-trimethyl-ethane-1,2-diamine | | |
| (1025) | 1-[1-(4-Bromophenyl)-5-phenyl-1H- | 2.50E-07 | n.d. |
| | pyrazol-4-yl]-4-methylpiperazine | | |
| (1026) | 1-(Biphenyl-4-yltrifluoromethyl-1H- | 2.60E-07 | 1.00E-06 |
| | pyrazol-4-ylmethyl)-4-methylpiperazine | | |
| (1027) | 1-[5-(2-Chlorophenyl)-1-(4′-fluoro- | 2.60E-07 | 1.00E-06 |
| | biphenyl-4-yl)-1H-pyrazol-4-ylmethyl]- | | |
| | 4-cyclopentylpiperazine | | |
| (1028) | {1-[1-Biphenyl-4-yl-5-(2-fluorophenyl)- | 2.70E-07 | n.d. |
| | 1H-pyrazol-4-ylmethyl]piperidin-2- | | |
| | ylmethyl}-diethylamine | | |
| (1029) | 1-[5-(2-Fluorophenyl)-1-(4- | 2.70E-07 | 4.90E-07 |
| | trifluoromethoxy-phenyl)-1H-pyrazol-4- | | |
| | ylmethyl]-4-methyl-piperazine | | |
| (1030) | N-(2-Hydroxyethyl)-1-[1-biphenyl-4- | 2.70E-07 | n.d. |
| | yl-5-(2-fluorophenyl)-1H-pyrazol-4- | | |
| | ylmethyl]piperidine-4-carboxamide | | |
| (1031) | 2-[1-Biphenyl-4-yl-5-(2-fluorophenyl)- | 2.80E-07 | n.d. |
| | 1H-pyrazol-4-ylmethyl]-1,3,4,6,7,11b- | | |
| | hexahydro-2H-pyrazino[2,1-a] | | |
| | isoquinoline | | |
| (1032) | 2-{4-[1-Biphenyl-4-yl-5-(2-fluoro- | 2.80E-07 | n.d. |
| | phenyl)-1H-pyrazol-4-ylmethyl] | | |
| | piperazin-1-yl}ethanol | | |
| (1033) | 1-[1-Biphenyl-4-yl-5-(2-fluorophenyl)- | 2.80E-07 | n.d. |
| | 1H-pyrazol-4-ylmethyl]-4- | | |
| | ethylpiperidin-4-ol | | |
| (1034) | {[1-Biphenyl-4-yl-5-(2-fluorophenyl)- | 2.80E-07 | n.d. |
| | 1H-pyrazol-4-ylmethyl]amino} | | |
| | acetonitrile | | |
| (1035) | 1-(1-Biphenyl-4-yl-5-pyridin-3-yl-1H- | 2.90E-07 | 1.00E-06 |
| | pyrazol-4-ylmethyl)-4-methylpiperazine | | |
| (1036) | (1-Benzylpyrrolidin-3-yl)-[1-biphenyl- | 3.10E-07 | n.d. |
| | 4-yl-5-(2-fluorophenyl)-1H-pyrazol- | | |
| | 4-ylmethyl]amine | | |
| (1037) | 1-[5-(4-Chlorophenyl)-1-(4′-fluoro- | 3.10E-07 | 1.00E-06 |
| | biphenyl-4-yl)-1H-pyrazol-4-ylmethyl]- | | |
| | 4-ethylpiperazine | | |
| (1038) | 2-{4-[1-Biphenyl-4-yl-5-(2-fluoro- | 3.11E-07 | n.d. |
| | phenyl)-1H-pyrazol-4-ylmethyl] | | |
| | piperazin-1-yl}-1-pyrrolidin- | | |
| | 1-ylethanone | | |
| (1039) | Benzyl-[1-biphenyl-4-yl-5-(2-fluoro- | 3.20E-07 | n.d. |
| | phenyl)-1H-pyrazol-4-ylmethyl] | | |
| | ethylamine | | |
| (1040) | 1-[1-(4′-Fluorobiphenyl-4-yl)-5-(4- | 3.20E-07 | n.d. |
| | methoxy-phenyl)-1H-pyrazol-4- | | |
| | ylmethyl]-4-isopropyl-piperazine | | |
| (1041) | (3-Azabicyclo[3.1.0]hex-6-yl)-[1- | 3.20E-07 | n.d. |
| | biphenyl-4-yl-5-(2-fluorophenyl)-1H- | | |
| | pyrazol-4-ylmethyl]-amine | | |
| (1042) | 2-{4-[1-Biphenyl-4-yl-5-(2-fluoro- | 3.60E-07 | n.d. |
| | phenyl)-1H-pyrazol-4-ylmethyl] | | |
| | piperazin-1-yl}acetamide | | |
| (1043) | 1-[1-Biphenyl-4-yl-5-(2-fluorophenyl)- | 3.60E-07 | n.d. |
| | 1H-pyrazol-4-yl]-1-morpholin-4- | | |
| | ylmethanone | | |
| (1044) | [1-(2′,5′-Difluorobiphenyl-4-yl)-5- | 3.70E-07 | n.d. |
| | (2-fluoro-phenyl)-1H-pyrazol-4- | | |
| | ylmethyl](tetrahydro-furan-2- | | |
| | ylmethyl)amine | | |
| (1045) | 2-{[1-Biphenyl-4-yl-5-(2-fluoro- | 3.70E-07 | n.d. |
| | phenyl)-1H-pyrazol-4-ylmethyl] | | |
| | amino}-3-(3H-imidazol-4- | | |
| | yl)propan-1-ol | | |
| (1046) | [1-Biphenyl-4-yl-5-(2-fluorophenyl)- | 3.70E-07 | n.d. |
| | 1H-pyrazol-4-ylmethyl]-(5- | | |
| | methylthiazol-2-yl)amine | | |
| (1047) | 1-[1-Biphenyl-4-yl-5-(2-fluorophenyl)- | 3.80E-07 | n.d. |
| | 1H-pyrazol-4-ylmethyl]-4-thiophen-3- | | |
| | ylmethyl-piperazine | | |
| (1048) | [5-(2-Chlorophenyl)-1-(4′-fluoro- | 3.80E-07 | n.d. |
| | biphenyl-4-yl)-1H-pyrazol-4-yl] | | |
| | methanol | | |
| (1049) | 1-[1-(4′-Chlorobiphenyl-3-yl)-5-(2- | 3.90E-07 | n.d. |
| | fluoro-phenyl)-1H-pyrazol-4-ylmethyl] | | |
| | -4-methyl-piperazine | | |
| (1050) | 1-[5-(2-Fluorophenyl)-1-(5-trifluoro- | 3.90E-07 | n.d. |
| | methylpyridin-2-yl)-1H-pyrazol-4- | | |
| | ylmethyl]pyrrolidin-3-ol | | |
| (1051) | 8-[1-Biphenyl-4-yl-5-(2-fluorophenyl)- | 4.00E-07 | 1.00E-06 |
| | 1H-pyrazol-4-ylmethyl]-1-phenyl-1,3,8- | | |
| | triaza-spiro[4.5]decan-4-one | | |
| (1052) | 1-[5-(3,5-Dichlorophenyl)-1-(4′-fluoro- | 4.00E-07 | nd. |
| | biphenyl-4-yl)-1H-pyrazol-4-ylmethyl]- | | |
| | 4-methyl-piperazine | | |
| (1053) | 1-[1-Biphenyl-4-yl-5-(2-fluorophenyl)- | 4.30E-07 | n.d. |
| | 1H-pyrazol-4-ylmethyl]piperidin-4-ol | | |
| (1054) | 1-[5-(4-Chlorophenyl)-1-(4′-fluoro- | 4.40E-07 | 1.00E-06 |
| | biphenyl-4-yl)-1H-pyrazol-4-ylmethyl]- | | |
| | 4-isopropylpiperazine | | |
| (1055) | 1-[5-(2-Methoxyphenyl)-1-(4-thiophen- | 4.60E-07 | 3.00E-07 |
| | 3-yl-phenyl)-1H-pyrazol-4-ylmethyl]- | | |
| | 4-methyl-piperazine | | |
| (1056) | tert-Butyl 3-{[1-biphenyl-4-yl-5-(2- | 4.60E-07 | n.d. |
| | fluoro-phenyl)-1H-pyrazol-4-ylmethyl] | | |
| | amino}pyrrolidine-1-carboxylate | | |
| (1057) | 1-[5-(4-Chlorophenyl)-1-(4′-fluoro- | 4.60E-07 | 1.00E-06 |
| | biphenyl-4-yl)-1H-pyrazol-4-ylmethyl]- | | |
| | 4-methylpiperazine | | |
| (1058) | [1-Biphenyl-4-yl-5-(2-fluorophenyl)- | 4.70E-07 | n.d. |
| | 1H-pyrazol-4-ylmethyl] | | |
| | piperidin-4-ylamine | | |
| (1059) | 2-[1-Biphenyl-4-yl-5-(2-fluoro- | 4.70E-07 | n.d. |
| | phenyl)-1H-pyrazol-4-yl]ethanol | | |

The examples below relate to pharmaceutical compositions:

### EXAMPLE A

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄.2H₂O, 28.48 g of Na₂HPO₄. 12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced in a conventional manner into hard gelatine capsules in such a way that each capsule contains 20 mg of the active ingredient.

### EXAMPLE H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

### EXAMPLE I

### Inhalation Spray

14 g of active ingredient of the formula I are dissolved in 10 l of isotonic NaCl solution, and the solution is transferred into commercially available spray containers with pump mechanism. The solution can be sprayed into the mouth or nose. One spray shot (about 0.1 ml) corresponds to a dose of about 0.14 mg.

1. Use of the compounds of the formula I
in which 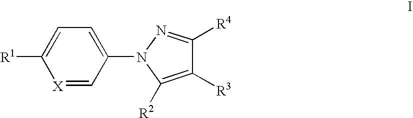
X denotes CH or N,
R¹ denotes H, A, Hal, (CF₂)ₙHet, (CH₂)ₙAr, cycloalkyl having 3 to 7 C atoms, CF₃, NO₂, CN, C(NH)NOH or OCF₃,
R² denotes (CH₂)ₙHet, (CH₂)ₙAr, cycloalkyl having 3 to 7 C atoms or CF₃,
R³, R⁴ denote H, (CH₂)ₙCO₂R⁵, (CH₂)ₙCOHet, (CH₂)ₙCON(R⁵)₂, (CH₂)ₙ000(CH₂)ₙHet, CHO, (CH₂)ₙOR⁵, (CH₂)ₙHet, (CH₂)ₙN(R⁵)₂, CH═N—OA, CH₂CH═N—OA, (CH₂)ₙNHOA, (CH₂)ₙN(R⁵)Het, (CH₂)ₙCH═N-Het, (CH₂)ₙ000R⁵, (CH₂)ₙN(R⁵)CH₂CH₂OR⁵, (CH₂)ₙN(R⁵)CH₂CH₂OCF₃, (CH₂)ₙN(R⁵)C(R⁵)H000R⁵, (CH₂)ₙN(R⁵)CH₂COHet, (CH₂)ₙN(R⁵)CH₂Het, (CH₂)ₙN(R⁵)CH₂CH₂Het, (CH₂)ₙN(R⁵)CH₂CH₂N(R⁵)CH₂000R⁵, (CH₂)ₙN(R⁵)CH₂CH₂OR⁵, (CH2)ₙN(R⁵)CH2CH₂N(R⁵)₂, CH═CH000R⁵, CH═CHCH₂NR⁵Het, CH═CHCH₂N(R⁵)₂, CH═CHCH₂0R⁵, CH═CHCH₂Het, (CH₂)ₙN(R⁵)Ar, (CH₂)ₙN(000R)COOR⁵, (CH₂)ₙN(CONH₂)000R⁵, (CH₂)ₙN(CONH₂)CONH₂, (CH₂)ₙN(CH₂000R)COOR⁵, (CH₂)ₙN(CH₂CONH₂)000R⁵, (CH₂)ₙN(CH₂CONH₂)CONH₂, (CH₂)ₙCHR⁵COR⁵, (CH₂)ₙCHR⁵000R⁵,
(CH₂)ₙCHR⁵CH20R⁵, where in each case one of the radicals R³ or R⁴ has the meaning H,
R⁵ denotes H or A
A denotes straight-chain or branched alkyl or cycloalkyl having 2 to 4 C atoms, having 1 to 10 C atoms, alkenyl having 2 to 10 C atoms, alkoxyalkyl having 2 to 10 C atoms or cycloalkyl having 4 to 7 C atoms, each of which is unsubstituted or substituted by Hal or CN,
Het preferably denotes a saturated, unsaturated or aromatic mono or bicyclic heterocyclic radical having 1 to 15 C atoms which is unsubstituted or mono- or polysubstituted by A and/or Hal or a linear radical having 1 to 15 C atoms containing one or two hetero atoms,
Ar denotes a phenyl radical which is unsubstituted or mono or polysubstituted by A and/or Hal, ORt, OOCR⁵, COORS, CON(R⁵)₂, CN, NO₂, NH₂, NHCOR⁵, CF₃ or SO2CH₃,
n denotes 0, 1, 2, 3, 4 or 5 and
Hal denotes F, Cl, Br or I,
and salts and solvates, enantiomers and racemates thereof for the preparation of a medicament for the treatment and prophylaxis of diseases which can be influenced by the binding of the compounds of the formula I to 5 HT receptors.

2. Use of compounds according to claim 1 and/or physiologically acceptable salts and solvates thereof for the preparation of a medicament having a 5-HT receptor-antagonistic action.

3. Use of compounds according to claim 1 and/or physiologically acceptable salts and solvates thereof for the preparation of a medicament having a 5-HT_{2A} receptor-antagonistic action.

4. Use of compounds of the formula I according to claim 1, and/or physiologically acceptable salts or solvates thereof for the preparation of a medicament for the prophylaxis and/or treatment of psychoses, neurological disorders, amyotrophic lateral sclerosis, eating disorders, such as bulimia, anorexia nervosa, of premenstrual syndrome and/or for positively influencing obsessive-compulsive disorder (OCD).

5. Use of the compounds of the formula I according to claim 1, in which R¹ denotes phenyl, 2-, 3- or 4-cyanophenyl, 2-, 3- or 4-fluorophenyl, 2-, 3- or 4-methyl-, ethyl-, n-propyl- or n-butylphenyl, 2,3-, 2,4-, 2,5-, 2,6, 3,4, 3,5- or 3,6-difluoro-, dichloro- or dicyanophenyl, 3,4,5-trifluorophenyl, 3,4,5-trimethoxy- or triethoxyphenyl, thiophen 2-yl or thiophen-3-yl or 1-, 2- or 3-pyrrolyl.

6. Use of the compounds of the formula I according to claim 1, in which R³ denotes (CH2)ₙCO₂R⁵, (CH2)ₙCO-Het, CHO, CH₂OR⁵, (CH2)ₙ-Het, (CH2)ₙN(R⁵)₂ or CH═N—OA, (CH₂)ₙN(R⁵)Het, (CH2)ₙN(R⁵)CH₂CH₂OR⁵, (CH2)ₙN(R⁵)CH₂Het, (CH2)ₙN(R⁵)CH2CH2Het, (CH2)nN(R⁵)CH2CH2N(R⁵)2, CH═CHCH₂NR⁵Het, CH═CHCH2N(R⁵)₂, CH═CHCH₂OR⁵, CH═CHCH₂Het or (CH2)ₙN(R⁵)Ar.

7. Use of the compounds of the formula I according to claim 1, in which R⁴ denotes H.

8. Use of the compounds of the formula I according to claim 1, in which R² denotes phenyl, 2-, 3- or 4-cyanophenyl, 2-, 3- or 4-fluorophenyl, 2-, 3- or 4-methyl-, ethyl-, n-propyl- or n-butylphenyl, 2,3-, 2,4-, 2,5-, 2,6-difluoro- or dicyanophenyl, thiophen-2-yl or thiophen-3-yl, 2-, 3- or 4-pyridyl, 2-, 4- or 5-oxazolyl, 2-, 4- or 5-thiazolyl, quinolinyl, isoquinolinyl, 2- or 4-pyridazyl, 2-, 4- or 5-pyrimidyl, 2- or 3-pyrazinyl, 2- or 3-furyl.

9. Use of the compounds of the formula I according to claim 1, in which X has the meaning CH.

10. Use of the compounds of the formula (a) to (o) according to claim 1:
[1-biphenyl-4-yl-5-(2-fluorophenyl)-1H-pyrazol-4-ylmethyl]-(4-methylpiperazin-1-yl)amine (a)
4-{2-[1-biphenyl-4-yl-5-(2-fluorophenyl)-1H-pyrazol-4-yl]-ethyl}morpholine (b)
4-{3-[1-biphenyl-4-yl-5-(2-fluorophenyl)-1H-pyrazol-4-yl]allyl}morpholine (c)
1-[1-biphenyl-4-yl-5-(2-fluorophenyl)-1H-pyrazol-4-yl-methyl]pyrrolidin-3-ol (d)
1-[1-(4′-fluorobiphenyl-4-yl)-5-(2-fluorophenyl)-1H-pyrazol-4-ylmethyl]-4-methylpiperazine (e)
1-[5-(2-fluorophenyl)-1-(4-thiophen-3-ylphenyl)-1H-pyrazol-4-ylmethyl]-4-methylpiperazine (f)
1-[5-furan-2-yl-1-(4-thiophen-3-ylphenyl)-1H-pyrazol-4-yl-methyl]-4-methylpiperazine (g)
N¹-[1-biphenyl-4-yl-5-(2-fluorophenyl)-1H-pyrazol-4-yl-methyl]ethane-1,2-diamine (h)
2-{[1-biphenyl-4-yl-5-(2-fluorophenyl)-1H-pyrazol-4-yl-methyl]amino}ethanol (i)
[1-biphenyl-4-yl-5-(2-fluorophenyl)-1H-pyrazol-4-ylmethyl]-(2-methoxyethyl)amine 0)
2-{[1-biphenyl-4-yl-5-(2-fluorophenyl)-1H-pyrazol-4-yl-methyl]methylamino}ethanol (k)
1-[1-biphenyl-4-yl-5-(2-fluorophenyl)-1H-pyrazol-4-yl-methyl]-4-methyl-[1,4]diazepam (l)
1-[1-(4′-fluorobiphenyl-4-yl)-5-phenyl-1H-pyrazol-4-yl-methyl]-4-methylpiperazine (m)
1-[5-(2-fluorophenyl)-1-(4-pyrrol-1-ylphenyl)-1H-pyrazol-4-ylmethyl]-4-methylpiperazine (n)
[1-biphenyl-4-yl-5-(2-fluorophenyl)-1H-pyrazol-4-ylmethyl]-methyl-(1-methylpyrrolidin-3-yl)amine (o)
and salts and solvates thereof.
